(19) United States
(12) Patent Application Publication
(10) Pub. No.: US 2006/0241288 A1
(43) Pub. Date: Oct. 26, 2006

(54) Extracellular aspergillus polypeptides

(75) Inventors: Andrew Roche, Clongriffin (IE); Martin Chr. Hansen, Odense (DK); Inge D. Villsen, Odense (DK); Petra Schrotz-King, Odense (DE); Jeannette Henningsen, Odense (DK); Trine Louise Lund Jørgensen, Odense (DK)

Correspondence Address: SPECKMAN LAW GROUP PLLC, 1201 THIRD AVENUE, SUITE 330, SEATTLE, WA, 98101, US

(73) Assignee: ACE BioSciences A/S, Odense (DK)

(21) Appl. No.: 10/559,952

(22) PCT Filed: Jun. 10, 2004
(86) PCT No.: PCT/DK04/00407

§ 371 Date: Jun. 19, 2006

Related U.S. Application Data
(60) Provisional application No. 60/477,355, filed on Jun. 11, 2003.
(60) Provisional application No. 60/482,451, filed on Jun. 26, 2003.

(30) Foreign Application Priority Data
Jun. 10, 2003 (DK) PA 2003 00862
Jun. 26, 2003 (DK) PA 2003 00968

(51) Int. Cl. C07K 16/14

(52) U.S. Cl. 530/388.5

(57) ABSTRACT

The present invention relates to extracellular polypeptides of Aspergillus fumigatus, to fragments of these polypeptides, to compositions comprising such polypeptides and fragments and to exposed domains and epitopes of these polypeptides. The invention also relates to the use of these polypeptides and fragments for immunisation and for production of antibodies, and to antibodies that recognise and bind the polypeptides. Furthermore, the invention relates to methods of identifying binding partners and inhibitors, and to methods of preventing, treating and diagnosing Aspergillus infections.

### CROSS REFERENCES TO RELATED APPLICATIONS

This application claims priority under 35 U.S.C. §365(a) to International Patent Application No. PCT/DK2004/000407, filed Jun. 10, 2004, which claims priority to Danish Patent Application No. PA 2003 00862, filed Jun. 10, 2003, Danish Patent Application No. PA 2003 00968, filed Jun. 26, 2003, U.S. Provisional Application No. 60/477,355, filed Jun. 11, 2003, and U.S. Provisional Application No. 60/482,451, filed Jun. 26, 2003. This application also claims priority under 35 U.S.C. §120 to International Patent Application No. PCT/DK2004/000407, filed Jun. 10, 2004, which claims priority to Danish Patent Application No. PA 2003 00862, filed Jun. 10, 2003, Danish Patent Application No. PA 2003 00968, filed Jun. 26, 2003, U.S. Provisional Application No. 60/477,355, filed Jun. 11, 2003, and U.S. Provisional Application No. 60/482,451, filed Jun. 26, 2003.

### FIELD OF THE INVENTION

The present invention relates to extracellular polypeptides of Aspergillus fumigatus, to fragments of these polypeptides, to compositions comprising such polypeptides and fragments and to exposed domains and epitopes of these polypeptides. The invention also relates to the use of these polypeptides and fragments for immunisation and for production of antibodies, and to antibodies that recognise and bind the polypeptides. Furthermore, the invention relates to methods of identifying binding partners and inhibitors, and to methods of diagnosing Aspergillus infections.

### BACKGROUND OF THE INVENTION

The rise of diseases that attack the immune system, such as AIDS, and medical treatments that depress the immune system, such as cancer chemotherapy or organ transplantation, have resulted in an increase in the death rate caused by fungal infections. Since the mid-1980's, fungal pathogens have begun to rival their bacterial counterparts in many different medical settings. Species of the Aspergillus family account for a substantial number of these fungal infections and in particular Aspergillus fumigatus has emerged world-wide as a frequent cause of nosocomial infection in virtually every major medical centre. For almost 30 years, amphotericin B was the only drug approved for treating serious fungal infections despite significant kidney toxicity. Azoles were introduced in the 1980s for treating the most common fungal pathogen, Candida albicans, which is responsible for approximately 50% of fungal infections. Widespread use of azoles encouraged the development of resistant strains to this drug. Unfortunately, most currently marketed azoles are largely ineffective against the more severe forms of fungal disease, such as infections caused by Aspergillus. The increase in drug-resistant strains of fungal pathogens further underscores the need for new antimicrobial treatments.

Aspergillus fumigatus is a saprophytic fungus found ubiquitously in the environment, particularly in soil and in water and may be readily found in very large numbers in hay, grain and decaying organic matter. Aspergillus fumigatus plays an essential role in recycling environmental carbon and nitrogen. Reservoirs in hospitals and other institutions include unfiltered air, ventilation systems, contaminated dust during construction work, carpeting, food, ornamental plants and water and water supply systems. It is generally believed that aspergillosis occurs as a consequence of the exogenous acquisition of spores; they are small enough (2.5-3.0 μm) to reach the alveoli upon inhalation and hardy enough to survive for prolonged periods in fomites. It remains unclear what the size of the infectious inoculum needs to be, although this probably depends upon the immunological status of the host. There are around 600 recognised species, but only a small number have been identified as pathogenic. Among these A. fumigatus which causes over 80% of human infections caused by Aspergillus species. A. fumigatus is an opportunistic pathogen and normal individuals are not susceptible to disease except after inhalation of large quantities of spores. Aspergillus can cause illness in at least three ways: an allergic reaction in asthmatics (allergic aspergillosis); a colonisation in scarred lung tissue (aspergilloma); and an invasive infection with pneumonia which can affect the heart, lungs, brain and kidneys (invasive aspergillosis).

Allergic Aspergillosis

In the first type of aspergillosis illness, people with allergic asthma or genetic predisposition may develop this form of asthma upon becoming sensitised to Aspergillus species. Asthmatics may find their asthmatic condition aggravated upon exposure to A. fumigatus. Some people develop allergic bronchopulmonary aspergillosis (ABPA), a condition in which Aspergillus spores germinate and the resultant mycelial growth can potentially block the bronchi. Patients may cough up small, brown plugs of mycelia. There is no invasion of tissue. However, the patient may suffer lung fibrosis and may, over time, become more susceptible to other lung diseases. ABPA is currently the most severe allergic pulmonary complication caused by Aspergillus species. It occurs in patients suffering from atopic asthma or cystic fibrosis. Another disease entity, related to ABPA only because it is immune-mediated, hypersensitivity pneumonitis (also called extrinsic allergic alveolitis) is often associated with repeated exposure to an identified—often occupational—source of high levels of antigen.

Aspergilloma

Aspergilloma, commonly referred to as “fungus ball,” occurs in pre-existing pulmonary cavities that were caused by tuberculosis, sarcoidosis, or other bullous lung disorders and in chronically obstructed paranasal sinuses.

Invasive Aspergillosis (IA)

Invasive aspergillosis (IA) is seen in people whose normal immune systems are compromised by other serious diseases such as leukaemia, lymphoma, carcinoma, tuberculosis, emphysema, diabetes, HIV/AIDS or by use of immunosuppressive drugs (often used in connection with organ or bone marrow transplant operations); or by large doses of corticosteroids. In IA, there is an actual invasion of lung tissue or skin. Infection can also occur in many organs or tissues, e.g. heart, liver, eye, nose, ear and skeletal muscle. Pathologically invasive infections show clear invasion of the underlying tissue, eventually leading to bloodstream dissemination or contiguous spread to adjacent structures. The prognosis for IA is serious illness and death.

A fourfold increase in IA has been observed in the last 12 years. In 1992, IA was responsible for approximately 30% of fungal infections in patients dying of cancer, and it is estimated that IA occurs in 10 to 25% of all leukaemia patients, in whom the mortality rate is 80 to 90%, even when treated. The average incidence of IA is estimated to be 5 to 25% in patients with acute leukaemia, 5 to 10% after allogenic bone marrow transplantation (BMT), and 0.5 to 5% after cytotoxic treatment of blood diseases or autologous BMT and solid-organ transplantation. IA which follows solid-organ transplantation is most common in heart-lung transplant patients (19 to 26%) and is found, in decreasing order, in liver, heart, lung, and kidney recipients (1 to 10%) (Patel and Paya, 1997, Clin. Microbiol. Rev. 10: 86-124). IA also occurs in patients with nonhematogenous underlying conditions; it is increasingly reported in AIDS patients 1 to 12%) (Denning et al., 1991, N. Engl. J. Med. 324: 654-662) and is also a common infectious complication of chronic granulomatous disease (25 to 40%) Four types of IA have been described (Denning, 1998, Clin, Infect. Dis. 26: 781-805): (i) acute or chronic pulmonary aspergillosis, the most common form of IA; (ii) tracheobronchitis and obstructive bronchial disease with various degrees of invasion of the mucosa and cartilage as well as pseudomembrane formation, seen predominantly in AIDS patients; (iii) acute invasive rhinosinusitis; and (iv) disseminated disease commonly involving the brain (10 to 40% in BMT patients) and other organs (for example, the skin, kidneys, heart, and eyes).

Diagnosis of Aspergillus Infections

Unlike bacterial infections, cultures from blood or cerebrospinal fluid and other sterile body fluids—are rarely positive for Aspergillus species, even in patients with endocarditis and disseminated disease. Given the ubiquitous nature of the spores, recovering Aspergillus from cultures of the respiratory tract does not discriminate between genuine infection, colonization or contamination. A number of clinical findings may trigger a diagnosis of invasive aspergillosis, such as neutropenic fever not responding to broad-spectrum antibiotics, the development of new pulmonary infiltrates on chest X-ray and the presence of clinical signs suggestive of invasive mycosis (e.g. pleuritic chest pain, hemoptysis, etc.). Unfortunately, most of these triggers have low predictive value. Therefore, the only way to reach a precise and early diagnosis is to make intense efforts to collect specimens for culture and histopathological examination (by biopsy or needle aspiration). However, this gold standard approach involves aggressive procedures (open lung biopsy, brain biopsy, etc.) that are often precluded by cytopenia or by the critical condition of the patient. Hence, definitive diagnosis is infrequently made before fungal proliferation becomes overwhelming and therapy may no longer be successful.

The detection of anti-Aspergillus antibodies has no place in the diagnosis of aspergillosis in neutropenic patients and hematopoietic stem cell transplant recipients because these populations are not capable of mounting an adequate antibody response. Diagnostic tools used at the moment are galactomannan detection (a major cell wall constituent released during growth), high-resolution pulmonary CT-scanning and detection of aspergillar DNA. Obtaining both high sensitivity and high selectivity remains a problem, and there is a need for novel reliable diagnostic markers.

Currently Available Anti-Aspergillus Agents

The antifungal armamentarium that is currently available for the treatment of invasive aspergillosis is limited in number. It includes:

1. The polyene macrolide, amphotericin-B and its lipid-based formulations;

2. the triazole, itraconazole;

3. the fluorinated pyrimidine, 5-fluorocytosine; and

4. the allylamine, terbinafine.

The lack of a highly selective fungal target, not present in other eukaryotic cells, has for a long time precluded the development of new agents. With the exception of 5-fluorocytosine, all available agents act by interfering with the structural or functional integrity of the fungal plasma membrane, either by physical disruption or by blocking the biosynthesis of membrane sterols. This strategy remains far from ideal since the non-selective nature of the therapeutic target results in concomitant cross-inhibition (or toxicity) in mammalian cells.

Treatment with antifungal drugs such as amphotericin-B and/or itraconazole involves many difficulties. Amphotericin-B, flucytosine and itraconazole are associated with low success rates and are hampered by serious infusion- or drug-related toxicity, by hazardous drug-drug interactions, by pharmacokinetic problems and by the development of resistance. Amphotericin-B has to be given by vein in large doses. In some patients the treatment can damage kidney and other organs. The overall success rate of Amphotericin-B therapy for IA is 34%. In addition, most IA cases occur in spite of empirical administration of Amphotericin-B in response to a fever unresponsive to antibacterial agents. Itraconazole is generally given orally (also in large doses, e.g. at least 400 mg daily) and has been used for many years as a treatment, but even so, mortality is still as high as 85%.

Vaccination

Vaccination may be another approach for combating Aspergillus infections. As explained above, IA is a severe problem for immunocompromised patients and especially in neutropenic patients, who have lost all their acquired immune response and are virtually without memory, Aspergillus infection is lethal in most cases. It seems that vaccination of these patients prior to immune suppression would not be a viable strategy. However, vaccination of a bone marrow donor could assist in the clearance of infection post donation. Also passive immunisation with immunoglobulins may be an option. Until now there have been no extensive preclinical and/or clinical data available concerning the efficacy of specific immunoglobulins. However, there are reports from invasive Aspergillosis studies in mice that show that active vaccination has influence on their mortality rate (Ito and Lyons (2002) J. Infect. Dis. 186, 869-871).

Targets

As A. fumigatus is becoming a major fungal pathogen of humans there is an urgent need for identification of suitable biochemical targets in A. fumigatus and for the discovery and development of new effective antifungal agents active against such biochemical targets. Recently, the A. fumigatus genome was analysed by random shotgun DNA sequencing. By sequence comparison with Candida albicans genes known to be essential for survival, a large number of potentially essential A. fumigatus genes was identified (WO 02/086090). Such genes may potentially be interesting drug targets, but information on structure, function or cellular localisation of most of the encoded gene products is not yet available.

### SUMMARY OF THE INVENTION

In a main aspect, the present application relates to extracellular polypeptides of A. fumigatus. In the context of this application, an ‘extracellular polypeptide’ is defined as a polypeptide which is entirely or partially (i.e. part of the polypeptide chain or part of the population of polypeptide molecules) localised outside the plasma membrane of a fungal cell. Thus, extracellular polypeptides include plasma-membrane polypeptides which have extracellular parts, cell-wall polypeptides, periplasmic polypeptides, secreted polypeptides and all other polypeptides that are fully or partially exposed to or released into the space outside the plasma membrane. Extracellular polypeptides furthermore include all polypeptides or polypeptide fragments that can be found in cell-wall, cell-surface-exposed and diffusate fractions isolated as described herein.

Extracellular polypeptides are attractive targets for antifungal therapy and/or diagnosis since the exposure of such polypeptides to the extracellular space means that compounds that interact with these peptides (e.g. compounds used to prevent, treat or diagnose fungal infections) often do not need to pass through the plasma membrane to be effective. This is a considerable advantage as the plasma membrane constitutes a major barrier for most types of compounds.

Extracellular localisation of a fungal protein can usually not be predicted from its amino-acid sequence. The presence of a signal sequence mediating entrance of protein into the secretory pathway can be predicted with a high degree of certainty, but many proteins carrying such sequences remain intracellular, in compartments such as the endoplasmic reticulum, the Golgi complex, endosomes and lysosomes. Very little is known about sorting signals in A. fumigatus.

In principle, localisation of A. fumigatus proteins could be inferred from a known localisation of homologous proteins in other fungi, such as Saccharomyces cerevisiae or the pathogenic yeast Candida albicans, which are much better characterised than A. fumigatus. However, in practice, such predictions are highly uncertain. A recently performed genetic screening for putative exported C. albicans proteins identified a number of such proteins whose closest homologue was an intracellular protein (Monteoliva et al. (2002) Eukaryotic Cell 1, 514-525). Thus, even with the genome sequence of A. fumigatus available, it is not easy to predict which polypeptides can be found extracellularly.

The inventors have isolated and analysed cell-wall-, cell-surface-exposed- and diffusate fractions of A. fumigatus and thus determined extracellular localisation of the following polypeptides:

1. The polypeptide set forth in SEQ ID NO:1. This polypeptide has not previously been detected in A. fumigatus, as it was only previously proposed as a putative gene product on the basis of a nucleotide sequence. It is herein proposed to name this polypeptide CssI, for Conidial Surface and Secreted protein I.

2. Hydrophobin (SEQ ID NO:2). Previously described in Parta et al. (1994) Infect. Immun. 62, 4389-4395.

3. GAPDH-B, glyceraldehyde 3-phosphate dehydrogenase (SEQ ID NO:3). A 172 amino-acid fragment of this sequence has been described in the NCBI database under accession number AAL25819 (SEQ ID NO:35). However, the full-length polypeptide has not been described previously.

4. enolase (SEQ ID NO: 4). Described in the NCBI database under accession number AAK49451.

5. catalase B (SEQ ID NO:5). Described in the NCBI database under accession number AAB71223 and in Calera et al. (1997) Infect. Immun. 65, 4718-4724.

6. catalase A (SEQ ID NO:6). Described in the NCBI database under accession number U87630.

7. isopropylmalate dehydrogenase B (IMDH B) (SEQ ID NO: 36). This A. fumigatus polypeptide has not been described previously.

For several of these polypeptides, no localisation was known previously. For all polypeptides, novel polypeptide fragments that are useful in prevention, therapy or diagnosis of Aspergillus infections were identified by the inventors. Several of these fragments are relatively accessible from the extracellular space or released into it.

In a first main aspect, the invention relates to the polypeptide set forth in SEQ ID NO:3 and variants and fragments thereof, with the proviso that the fragment does not consist of the sequence set forth in SEQ ID NO:35.

In another main aspect, the invention relates to the polypeptide set forth in SEQ ID NO: 36 and variants and fragments thereof.

In a further main aspect, the invention relates to polypeptide fragments which are derived from the polypeptides set forth in SEQ ID NOs: 1-6 and 36, and comprise one or more amino-acid residues from the sequences set forth in SEQ ID NOs:7-34 and 37. The invention also relates to variants of these polypeptide fragments.

The invention also relates to exposed domains and epitopes which are comprised within or comprise part of the polypeptides or polypeptide fragments of the invention.

Furthermore, the invention relates to compositions comprising one or more extracellular Aspergillus polypeptides or polypeptide fragments of the invention.

The techniques that were used by the inventors in the identification of polypeptides in the different cell-wall-, cell-surface-exposed and/or diffusate fractions favour identification of highly expressed proteins. Thus, the polypeptides that were identified are relatively abundant. This, added to the determination that they are exposed to the extracellular environment of the cell, make them highly suitable as biochemical targets or diagnostic markers. Thus, the identification of these polypeptides in these fractions by the inventors formed the basis for the development of methods aimed at prevention, treatment and/or diagnosis of Aspergillus infections.

Accordingly, in a main aspect, the invention relates to use of polypeptides or fragments of the invention for generating a medicament. Preferably, a medicament that can be used for the immunisation or vaccination of a mammal, preferably a human being, preferably to generate a protective immune response.

Furthermore, in another main aspect, the invention relates to methods of raising antibodies against these polypeptides or fragments thereof in non-human mammals.

The invention also, in a further main aspect, relates to antibodies capable of binding an extracellular Aspergillus fumigatus polypeptide selected from the group consisting of isopropylmalate dehydrogenase B (SEQ ID NO:36), CssI (SEQ ID NO:1), hydrophobin (SEQ ID NO:2), GAPDH-B (SEQ ID NO:3), and catalase A (SEQ ID NO:6). Use of such antibodies for the manufacture of a medicament for treatment or prevention of infection with Aspergillus is also an aspect of the invention. Thus, the invention also relates to pharmaceutical compositions comprising antibodies of the invention and a pharmaceutically-acceptable carrier.

Moreover, the invention relates to methods of treating or preventing Aspergillus fumigatus infections and/or other fungal infections comprising the step of administering antibodies of the invention to an individual in need thereof.

Furthermore, the invention relates to methods for screening for binding partners and/or inhibitors of these extracellular polypeptides, to methods for screening for antifungal agents and to methods aimed at diagnosing Aspergillus infections.

### BRIEF DESCRIPTION OF THE DRAWINGS

The present invention will be described in greater detail in the following detailed description, with reference to the accompanying drawings, wherein:

FIG. 1 shows the predicted full-length polypeptide sequences of CssI (A) (SEQ ID NO:1), hydrophobin (B) (SEQ ID NO:2), GAPDH-B (C) (SEQ ID NO:3), enolase (D) (SEQ ID NO:4), catalase B (E) (SEQ ID NO:5), catalase A (F) (SEQ ID NO:6), and isopropylmalate dehydrogenase B (G) (SEQ ID NO:36);

FIG. 2 shows the predicted antigenicity indices of CssI (A) and hydrophobin (B) residues, predicted according to Jameson and Wolf (1988);

FIG. 3 shows the alignment of the predicted protein sequences for GAPDH-A (AfA), GAPDH-B (AfB), and GAPDH-C (AfC) from Aspergillus fumigatus. Residues that are identical in all three proteins are presented on a dark background. Peptides of GAPDH-B that have been identified by MS have been underlined;

FIG. 4 shows silver stained SDS-PAGE of AfM lysate samples (lanes 2 and 7) added to affigel immunoaffinity columns prepared with (lanes 2-5) or without (lanes 6-10) anti-AfM IgG. Lanes 1 and 6 indicate the samples that were eluted from these columns after extensive washing steps (lanes 3-5 and 8-10). The proteins that are eluting at approximate weights of 97, 64 and 51 were all identified as IMDH B via mass spectrometry;

FIG. 5 shows phase contrast (A) and immunofluorescent micrographs (B) of AfM labelled with anti-AfM sera (C) and with secondary antibody alone (D);

FIG. 6 shows adhesion of AfC to lung epithelia demonstrating the ability of anti-AfM Fab fragments to reduce adhesion of AfC to A549 cells;

FIG. 7 shows a silver stained SDS-PAGE illustrating the steps involved in purification of IMDH B. Lane 1, MW standard; lane 2, recombinant IMDH B purified via a nickel sepharose column; lane 3, recombinant protein following purification via an S200 gel filtration column; lane 4, as with lane 4 but ten times the quantity of protein is added;

FIG. 8 shows a coomasie blue stained SDS-PAGE indicating the nickel sepharose purified recombinant IMDH B that was used to immunize rabbits (1); and the reactivity of preimmune serum (2), the first bleed post immunization (3) and the second bleed post immunization against the recombinant protein as detected via Western blotting;

FIG. 9 shows immunofluorescent micrographs of AfC and AfM from ATCC 46640 stained with Pre and Post immune IgG isolated from a rabbit immunized with IMDH B;

FIG. 10 shows pre-incubation of AfC with anti-IMDH B reactive post immune Fab fragments reduces AfC adhesion to A549 cells;

FIG. 11 shows pre-incubation of rIMDH B with A549 cells, followed by washing, reduces subsequent adherence of AfC in a linear manner;

FIG. 12 shows incubation of AfC in the presence of anti-IMDH B sera and normal complement (sample 2) results in reduced germination as compared to samples incubated in the presence of preimmune or unrelated sera (samples 5 and 8, respectively);

FIG. 13 shows incubation of AfC in the presence of anti-IMDH B IgG and normal complement results in reduced germination as compared to samples incubated in the presence of preimmune or anti-KLH IgG;

FIG. 14 shows an alignment of IMDH B 1 versus IMDH B 2;

FIG. 15 shows an immunofluorescent microscopy analysis of A. fumigatus ATCC 46640 performed with antisera raised against MAP molecules. AfC and AfM were stained with both pre- and post-immunisation sera;

FIG. 16 shows an immunofluorescent microscopy analysis performed with a clinical isolate using antisera raised against GAP-B-2. AfC were stained with both pre- and post-immunisation sera;

FIG. 17 shows western blot experiments illustrating the detection of (A) recombinant enolase with anti-his (lane 1) and anti-enolase (lane 3; ENO-2, see Table 3) antibodies, but not with pre immune serum from the animal used to produce anti-enolase sera (Lane 4); and (B) native enolase in the cell membrane of AfC by anti-enolase (ENO-2, see Table 3) antibodies (lane 6);

FIG. 18 shows an alignment of part of SEQ ID NO:36 with a homologous sequence from Candida albicans, derived from publicly available nucleotide sequences (contig19-10262 in the Ca-Assembly19.contigs);

FIG. 19 shows an alignment of part of SEQ ID NO:36 with Aspergillus nidulans homolog (ACCESSION: AnrP4374925—DESCRIPTION: LE3B_ASPNG 3-isopropylmalate dehydrogenase B (Beta-IPM dehydrogenase B) (IMDH B) (3-IPM-DH B) [Aspergillus nidulans FGSC A4] DBXREF: gi|40744045|gb|EAA63227.1);

FIG. 20 shows an alignment of part of SEQ ID NO:36 with Aspergillus oryzae homolog (ACCESSION: AnrP3711474—DESCRIPTION: hypothetical protein [Aspergillus oryzae] DBXREF: gi↑27901558|dbj|BAC55906.1);

FIG. 21 shows an alignment of part of SEQ ID NO:36 with Aspergillus nidulans homolog (ACCESSION: AnrP4379986—DESCRIPTION conserved hypothetical protein [Aspergillus nidulans FGSC A4] DBXREF: gi|40741202|gb|EAA60392.1);

FIG. 22 shows an alignment of part of SEQ ID NO:36 with a Coccidioides posadasii homolog: TIGR 222929/contig 1772 C. posadasii C735;

FIG. 23 shows an alignment of part of SEQ ID NO:36 with a Cryptococcus homolog. Ref.nr.: chr01 b.b3501.031220.c11;

FIG. 24 shows ClustalW of IMDH B homologs from FIGS. 18-22;

FIG. 25 shows a continuation of FIG. 24;

FIG. 26 shows Table 1, which illustrates the sequences of the peptides that were identified in the AfC fractions. Shown are, for each of the polypeptides (CssI, Hydrophobin, GAPDH, enolase, catalase (A+B) and isopropylmalate dehydrogenase B), the peptides that were identified in the three different fractions (diffusate, cell-surface-exposed, and cell wall), and the sequences of the peptides that were used for antibody production. X₁ is serine or alanine, X₂ is leucine or isoleucine;

FIG. 27 shows Table 2, which illustrates some biochemical characteristics for the full-length CssI polypeptide and for its N-terminal and its C-terminal half. ‘MW’ indicates molecular weight. ‘Residues’ indicates the number of residues;

FIG. 28 shows Table 3, which illustrated sequences of peptides chosen for the production of multiple antigenic peptides and antisera against selected target proteins; and

FIG. 29 shows Table 4, which illustrates an analysis of the ability of anti-IMDH B IgG to bind the surface of clinical isolates.

### DETAILED DESCRIPTION OF THE INVENTION

Definitions

A ‘fragment’ or ‘polypeptide fragment’ is defined as a non-full-length part of a polypeptide. The length of fragments may vary from 2 amino-acid residues to the full-length polypeptide minus one amino-acid residue. Preferably, fragments are less than 100 amino acids, such as less than 50 amino acids, e.g. less than 40 amino acids, such as less than 30 amino acids, e.g. less than 25 amino acids, such as less than 20 amino acids in length. Thus, for example fragments can be 2, 3, 4, 5, 6, 7, 8, 9, 10, 11, 12, 13, 14, 15, 16, 17, 18, 19 or 20 amino acids in length. In further embodiments, fragments comprise more than 5 amino acids, such as more than 9 amino acids, e.g. more than 10 amino acids, such as more than 15 amino acids, e.g. more than 20 amino acids, such as more than 30 amino acids. Expressed in another way, a fragment consists of a part of an amino-acid sequence which is less than 100% in length as compared to the full-length polypeptide. The length of the fragment can be less than 50%, such as less than 25%, such as less than 10% of the length of the full-length polypeptide. In other embodiments, the length of the fragment can be more than 5%, such as more than 10%, such as more than 25% of the length of the full-length polypeptide.

‘Variants’ of a given polypeptide or fragment are polypeptides or peptides that display a certain degree of sequence identity to said polypeptide or fragment. Variants preferably have at least 75% sequence identity, for example at least 80% sequence identity, such as at least 85% sequence identity, for example at least 90% sequence identity, such as at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with the given polypeptide or fragment. Sequence identity is determined with any of the algorithms GAP, BESTFIT, or FASTA in the Wisconsin Genetics Software Package Release 7.0, using default gap weights.

Preferred variants of a given polypeptide or fragment are variants in which all amino-acid substitutions between the variant and the given polypeptide or fragment are conservative substitutions. Conservative amino-acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine, a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino-acids substitution groups are: valine-leucine-isoleucine, phenylalanine-tyrosine, lysine-arginine, alanine-valine, and asparagine-glutamine.

Variants of a polypeptide or of a fragment thereof also include forms of the polypeptide or fragment wherein one or more amino acids have been deleted or inserted. Preferably, less than 5, such as less than 4, e.g. less than 3, such as less than 2, e.g. only one amino acid has been inserted or deleted. ‘Variants’ of a polypeptide or of a fragment thereof also include forms of these polypeptides or fragments modified by post-translational modifications of the amino-acid sequence. Also included are fusion proteins wherein the given polypeptide or fragment thereof has been fused (on the gene level or post-translationally) to another peptide or polypeptide.

An ‘exposed domain’ is defined as a part of a polypeptide that is exposed to the external environment. Secreted or released parts of polypeptides, which are not cell-associated, are examples of exposed domains. Exposed domains can also be found in polypeptides that are cell-associated. This can e.g. be determined by protease treatment as described herein in the Examples. i.e. an exposed domain of a polypeptide is a part of the polypeptide which is more accessible for proteases, such as trypsin or chymotrypsin, than other parts of the same polypeptide, and can be released from cellular association by protease treatment without disrupting the integrity of the cell. Surface exposure of a domain can also be determined using indirect immunofluorescence analysis, e.g. as described by Sanjuan et al. (1996) Microbiology 142, 2255-2262. Exposed domains of plasma-membrane-associated polypeptides are parts of such polypeptides that are located immediately adjacent to membrane-spanning regions and are located on the extracellular side of the plasma membrane. An exposed domain can be flanked on both or on only one side by a membrane-spanning region. Membrane-spanning regions can be predicted by a variety of methods, reviewed in Möller et al. (2001) Bioinformatics 17, 646-653. In a preferred embodiment, an exposed domain of a plasma-membrane-associated polypeptide is a part of a polypeptide located on the extracellular side of the plasma membrane, immediately adjacent to a membrane-spanning region (transmembrane helix) as predicted by the TMHMM program 2.0 (Krogh et al. (2001) J. Mol. Biol. 305, 567-580.

‘Epitope’ in this context covers any part of a polypeptide capable of being recognised by an antibody or functional equivalent thereof. Epitopes may consist of a stretch of consecutive amino-acid residues or of non-consecutive parts of a polypeptide. Typically, an epitope consists of 2-20 amino acids, such as 3-10 amino acids, preferably 3-8 amino acids, such as 3, 4, 5, 6, 7 or 8 amino acids.

‘Expression vector’ refers to a plasmid or phage or virus, for producing a polypeptide from a polynucleotide sequence. An expression vector comprises an expression construct, comprising an assembly of (1) a genetic element or elements having a regulatory role in gene expression, for example, promoters or enhancers, (2) a structural or coding sequence which is transcribed into mRNA and translated into protein, and which is operably linked to the elements of (1); and (3) appropriate transcription initiation and termination sequences.

‘Vaccine’ is used to indicate a composition capable of inducing a protective immune response against a microorganism in a human being or animal.

‘Protective immune response’ is used to indicate an immune response (humoral/antibody and/or cellular) inducing memory in an organism, resulting in the infectious agent, being met by a secondary rather than a primary response, thus reducing its impact on the host organism.

A ‘binding partner’ of a polypeptide refers to a molecule that can bind to said polypeptide. Such binding can be indirect, through another molecule, but is preferably direct. A binding partner can be any type of molecule, such as e.g. small hydrophobic molecules or e.g. a cellular or extracellular macromolecule, such as a protein, a carbohydrate or a nucleic acid. Preferred types of binding partners include antibodies, ligands or inhibitors.

The term ‘plurality’ indicates more than one, preferably more than 10.

‘Secreted’ in the present context refers to soluble polypeptides or fragments thereof that are not cell-associated and thus in principle diffuse freely in the surrounding medium. This includes fragments of polypeptides that are released from cellular association, for instance through proteolysis.

The term ‘indicator moiety’ covers a molecule or a complex of molecules that can be detected or generates a detectable signal. Preferably, the indicator moiety is an antibody or includes an antibody molecule. Thus, a preferred indicator moiety is an antibody coupled to a detectable substance. The detectable substance can in some embodiments comprise a second antibody.

‘Host-derived molecule’ or ‘host molecule’ refers to a molecule which is normally found in a host organism that can be infected with A. fumigatus. A host-derived molecule is preferably a host polypeptide, preferably a human polypeptide. Examples of host-derived molecules that interact with pathogenic fungi are serum albumin and transferrin, fibrinogen, complement fragment C3d, complement fragment iC3b, laminin, fibronectin, entactin, vitronectin, mannan adhesins, epithelial binding lectin-like protein, and agglutinin-like proteins.

The term ‘antibodies’ when used herein is intended to cover antibodies as well as functional equivalents thereof. Thus, this includes polyclonal antibodies, monoclonal antibodies (mabs)) derived from any species including but not limited to mouse, rat, hamster, rabbit and lama, human, humanised or chimeric antibodies, single-chain antibodies, and also Fab fragments, F(ab′)₂ fragments, fragments produced by a Fab expression library, anti-idiotypic antibodies, hybrids comprising antibody fragments, and epitope-binding fragments of any of the these. The term also includes mixtures of monoclonal antibodies.

‘Isolated’ used in connection with polypeptides and polynucleotides disclosed herein refers to these having been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes.

Polypeptides of the Invention

Fragments of Extracellular Aspergillus Polypeptides

The analysis of three different Aspergillus fractions (diffusate, cell-surface-exposed and cell wall) that was performed by the inventors led to the identification of different types and numbers of fragments from the same polypeptides for each of the fractions. For example, as is described in Example 1, five CssI peptides were identified in the cell-wall fraction while only one was identified in diffusate and cell-surface-exposed fractions. This difference may indicate structural features of the protein. Without being limited to a specific theory, a possible explanation for this is that a portion of CssI can be cleaved from the cell wall, releasing one part of the protein into the surrounding milieu, while the remainder of the protein remains embedded in the cell wall. Similarly, although other explanations are possible, the fact that only one peptide is detected in cell-surface fractions may suggest that an area of the protein comprising that peptide is exposed while the remainder of the protein is not. Even regions of a polypeptide which are not embedded in other cellular structures such as the cell wall, may still contain parts that are more accessible than other parts. For instance, the surface of a polypeptide may be more accessible than parts of the polypeptide which are buried within a tertiary protein structure. Protease treatment may also identify such protein surface regions.

Thus, the inventors have identified protein regions of particular interest. Exposed domains are, due to their accessibility, particularly attractive targets for diagnosis or for antifungal treatment. Moreover, exposed polypeptide fragments or domains are likely to contribute to, or comprise epitopes, and thus be highly suitable for antibody recognition. For many of the applications described below, it can be advantageous to work with fragments that are larger than the ones that were identified by the inventors. This can in particular be the case for methods of identifying binding partners and methods for raising antibodies, such as immunisation, which sometimes do not work well with small fragments.

In a main aspect, the invention relates to fragments of extracellular Aspergillus polypeptides that comprise exposed domains and/or epitopes. The invention also relates to the full-length GAPDH-B polypeptide (SEQ ID NO:3) and to the full-length isopropylmalate dehydrogenase B polypeptide (SEQ ID NO:36).

Accordingly, in a main aspect, the invention relates to an Aspergillus polypeptide selected from the group of fragments of SEQ ID NO:1 of less than 259 amino-acid residues in length, such as less than 200, preferably less than 150, such as less than 100, e.g. less than 50, such as less than 25 amino-acid residues in length comprising one or more residues of the amino-acid sequences set forth in SEQ ID NO:7, 8, 17, 26, 28, 29 and/or 30 and variants of said fragments; fragments of SEQ ID NO:2 of less than 106 amino-acid residues in length, such as less than 75, preferably less than 50, such as less than 25 residues in length comprising one or more residues of the amino-acid sequences set forth in SEQ ID NO:9, 10, 18 and/or 19 and variants of said fragments; polypeptides comprising SEQ ID NO:3, fragments thereof and variants thereof, with the proviso that if the polypeptide is a fragment of SEQ ID NO:3, that this fragment is not the fragment set forth in SEQ ID NO:35; fragments of SEQ ID NO:4 of less than 437 amino-acid residues in length, such as less than 200, preferably less than 100, such as less than 75, e.g. less than 50, such as less than 25 amino-acid residues in length comprising one or more residues of the amino-acid sequences set forth in SEQ ID NO:13, 14, 23, 24 and/or 25 and variants of said fragments; fragments of SEQ ID NO:5 of less than 727 amino-acid residues in length, e.g. less than 400, such as less than 200, preferably less than 100, such as less than 75, e.g. less than 50, such as less than 25 amino-acid residues in length comprising one or more residues of the amino-acid sequences set forth in SEQ ID NO:15, 16 and/or 27 and variants of said fragments; fragments of SEQ ID NO:6 of less than 748 amino-acid residues in length, e.g. less than 400, such as less than 200, preferably less than 100, such as less than 75, e.g. less than 50, such as less than 25 amino-acid residues in length comprising one or more residues of the amino-acid sequences set forth in SEQ ID NO:34 and variants of said fragments; and polypeptides comprising SEQ ID NO:36, fragments thereof and variants thereof.

In a preferred embodiment, the above fragments comprise one or more residues of the amino-acid sequences set forth in SEQ ID NOs: 7-27 and 37. In a more preferred embodiment, the fragments comprise one or more residues of the amino-acid sequences set forth in SEQ ID NOs: 7-16. In another more preferred embodiment, the fragments comprise one or more residues of the amino-acid sequences set forth in SEQ ID NOs: 17-25 and/or SEQ ID NO:14. In yet another more preferred embodiment, the fragments comprise one or more residues of the amino-acid sequences set forth in SEQ ID NO: 18, 19, 26, 27, and/or 37.

Further preferred polypeptides are fragments of SEQ ID NO:1 of less than 259 amino-acid residues in length, such as less than 200, preferably less than 150, such as less than 100, e.g. less than 50, such as less than 25 amino-acid residues in length comprising one or more residues of the amino-acid sequences set forth in SEQ ID NO:7, 8, 17, 26, 28, 29 and/or 30, such as one or more residues of the amino-acid sequences set forth in SEQ ID NO:7, e.g. one or more residues of the amino-acid sequence set forth in SEQ ID NO:8, such as one or more residues of the amino-acid sequences set forth in SEQ ID NO:17, e.g. one or more residues of the amino-acid sequence set forth in SEQ ID NO:26, such as one or more residues of the amino-acid sequences set forth in SEQ ID NO:28, e.g. one or more residues of the amino-acid sequence set forth in SEQ ID NO:29, such as one or more residues of the amino-acid sequences set forth in SEQ ID NO:30.

Further preferred polypeptides are fragments of SEQ ID NO:2 of less than 106 amino-acid residues in length, such as less than 75, preferably less than 50, such as less than 25 residues in length comprising one or more residues of the amino-acid sequences set forth in SEQ ID NO:9, 10, 18 and/or 19, such as one or more residues of the amino-acid sequences set forth in SEQ ID NO:9, e.g. one or more residues of the amino-acid sequence set forth in SEQ ID NO:10, such as one or more residues of the amino-acid sequences set forth in SEQ ID NO:18, e.g. one or more residues of the amino-acid sequence set forth in SEQ ID NO:19.

Preferred polypeptides include fragments of SEQ ID NO:3, with the proviso that if the polypeptide is a fragment of SEQ ID NO:3, that this fragment is not the fragment set forth in SEQ ID NO:35. Preferred are fragments of SEQ ID NO:3 of less than 171 amino acids in length, such as less than 150, preferably less than 100, such as less than 75, e.g. less than 50, such as less than 25 amino-acid residues in length comprising one or more residues of the amino-acid sequences set forth in SEQ ID NO:11, 12, 20, 21, 22, 31, 32 and/or 33, such as one or more residues of the amino-acid sequences set forth in SEQ ID NO:11, e.g. one or more residues of the amino-acid sequence set forth in SEQ ID NO:12, such as one or more residues of the amino-acid sequences set forth in SEQ ID NO:20, e.g. one or more residues of the amino-acid sequence set forth in SEQ ID NO:21, such as one or more residues of the amino-acid sequences set forth in SEQ ID NO:22, e.g. one or more residues of the amino-acid sequence set forth in SEQ ID NO:31, such as one or more residues of the amino-acid sequences set forth in SEQ ID NO:32, e.g. one or more residues of the amino-acid sequence set forth in SEQ ID NO:33. Other preferred fragments of SEQ ID NO:3 are fragment between 173 residues and 317 residues in length, comprising one or more residues of the amino-acid sequences set forth in SEQ ID NO:11, 12, 20, 21 and/or 22 or variants of said fragments.

Further preferred polypeptides are fragments of SEQ ID NO:4 of less than 437 amino-acid residues in length, such as less than 200, preferably less than 100, such as less than 75, e.g. less than 50, such as less than 25 amino-acid residues, in length comprising one or more residues of the amino-acid sequences set forth in SEQ ID NO:13, 14, 23, 24 and/or 25, such as one or more residues of the amino-acid sequences set forth in SEQ ID NO:13, e.g. one or more residues of the amino-acid sequence set forth in SEQ ID NO:14, such as one or more residues of the amino-acid sequences set forth in SEQ ID NO:23, e.g. one or more residues of the amino-acid sequence set forth in SEQ ID NO:24, such as one or more residues of the amino-acid sequences set forth in SEQ ID NO:25.

Further preferred polypeptides are fragments of SEQ ID NO:5 of less than 727 amino-acid residues in length, e.g. less than 400, such as less than 200, preferably less than 100, such as less than 75, e.g. less than 50, such as less than 25 amino-acid residues in length comprising one or more residues of the amino-acid sequences set forth in SEQ ID NO:15, 16 and/or 27, such as one or more residues of the amino-acid sequences set forth in SEQ ID NO:15, e.g. one or more residues of the amino-acid sequence set forth in SEQ ID NO:16, such as one or more residues of the amino-acid sequences set forth in SEQ ID NO:27.

Further preferred polypeptides are fragments of SEQ ID NO:6 of less than 748 amino-acid residues in length, e.g. less than 400, such as less than 200, preferably less than 100, such as less than 75, e.g. less than 50, such as less than 25 amino-acid residues in length comprising one or more residues of the amino-acid sequences set forth in SEQ ID NO:34.

Preferred polypeptides include polypeptides comprising or consisting of SEQ ID NO:36. Further preferred are fragments of SEQ ID NO:36, of less than 367 amino acid residues in length, such as less than 200, preferably less than 100, such as less than 75, e.g. less than 50, such as less than 25 amino-acid residues in length comprising one or more residues of the amino-acid sequences set forth in SEQ ID NO:37. In one embodiment, X₁ in SEQ ID NO:36 and SEQ ID NO: 37 is a serine. In another embodiment, X₁ in SEQ ID NO:36 and SEQ ID NO: 37 is an alanine. In a further embodiment, X₂ in SEQ ID NO:36 and SEQ ID NO: 37 is a leucine. In another embodiment, X₂ in SEQ ID NO:36 and SEQ ID NO: 37 is an isoleucine. Thus, different sequence embodiments for SEQ ID NO: 37 and the equivalent part of SEQ ID NO:36 include LAAELALR, LSAELALR, LAAEIALR, LSAEIALR.

Preferably, the above defined polypeptide fragments of SEQ ID NOs:1-6 and 36 comprise more than one residue of the specified amino-acid sequence, such as 2, 3, 4, 5, 6, 7, 8 or 9 residues of the specified amino-acid sequence. A non-limiting example of such a preferred fragment is a fragment of SEQ ID NO:1 comprising 9 residues of the amino-acid sequence set forth in SEQ ID NO:7. Most preferably, the above polypeptide fragments comprise all residues of the specified amino-acid sequence. A non-limiting example of a most preferred fragment is a fragment of SEQ ID NO:1 comprising all 16 residues of the amino-acid sequence set forth in SEQ ID NO:7.

In one embodiment, the polypeptide of the invention, preferably consists of an exposed domain, such as domains comprising an amino-acid sequence selected from the group of SEQ ID NOs: 7-34 and 37, preferably the group of SEQ ID NOs: 7-27 and 37, more preferably the group of SEQ ID NOs:17-27, SEQ ID NO:14 and SEQ ID NO:37, or variants thereof. An exposed domain may be determined as described above in the definition section.

Further preferred polypeptides consist of an epitope of a polypeptide selected from the group of SEQ ID NO:1-6 and 36, comprising at least one amino acid from a peptide selected from the group of SEQ ID NO: 7-27 and 37, and fragments or variants of said epitope. In one preferred embodiment, the amino acid residues of the epitope are consecutive residues from the polypeptide. In another preferred embodiment, the amino acid residues of the epitope are non-consecutive residues from the polypeptide. Further preferred embodiments include more than 1, such as more than 2, preferably more than 3, such as more than 4 consecutive or non-consecutive amino acids of the sequences of SEQ ID NO: 7-27 and 37. The invention also relates to use of such epitopes in any of the methods or preferred methods of the invention.

Fragments that consist or essentially consist of an amino-acid sequence selected from the group of SEQ ID NO: 7-34 and 37.

Preferred polypeptides of the invention are fragments of one of the polypeptides set forth in SEQ ID NO:1-6 and 36 that essentially consist of one of the fragments set forth in SEQ ID NO:7-34 and 37. ‘Essentially consists of’ is meant to indicate that the fragment comprises a substantial part of an amino-acid sequence selected from the group of SEQ ID NO:7-34 and 37 and in addition to that contains 10 or fewer flanking residues from the polypeptide on either or both (N-terminal and/or C-terminal) sides of the smaller fragment. A ‘substantial part’ herein means at least 2, such as at least 5 amino acids of any of the amino acid sequence set forth in SEQ ID NO:7-34 and 37. Such a fragment thus overlaps with the corresponding fragment selected from the group of SEQ ID NO:7-34 and 37. Preferably, the fragment that essentially consists of any of the amino-acid sequences set forth in SEQ ID NO:7-34 and 37 comprises the entire amino-acid sequence of that sequence. Thus, a preferred fragment of the invention is a fragment of one of the polypeptides set forth in SEQ ID NO:1-6 and 36 that comprises and essentially consists of one of the fragments set forth in SEQ ID NO:7-34 and 37. Such a fragment is thus larger than the corresponding fragment selected from the group of SEQ ID NO:7-34 and 37. ‘Comprises and essentially consists of’ is meant to indicate that the larger fragment comprises a smaller peptide selected from the group of SEQ ID NO:7-34 and 37 and in addition to that contains 10 or fewer flanking residues from the polypeptide on either or both (N-terminal and/or C-terminal) sides of the smaller fragment. Preferably, the larger fragment contains fewer than 8, such as fewer than 6, e.g. fewer than 4, e.g. fewer than 3, such as 2 or only 1 residue on one or both sides of the smaller fragment.

Most preferred polypeptides of the invention are fragments selected from the group of SEQ ID NO: 7-34 and 37. Thus, such most preferred polypeptides include any of the fragments from the group of fragments set forth in SEQ ID NO:7-34 and 37, such as the fragment set forth in SEQ ID NO:7, or the fragment set forth in SEQ ID NO:8, or the fragment set forth in SEQ ID NO:9, or the fragment set forth in SEQ ID NO:10, or the fragment set forth in SEQ ID NO:11, or the fragment set forth in SEQ ID NO:12, or the fragment set forth in SEQ ID NO:13, or the fragment set forth in SEQ ID NO:14, or the fragment set forth in SEQ ID NO:15, or the fragment set forth in SEQ ID NO:16, or the fragment set forth in SEQ ID NO:17, or the fragment set forth in SEQ ID NO:18, or the fragment set forth in SEQ ID NO:19, or the fragment set forth in SEQ ID NO:20, or the fragment set forth in SEQ ID NO:21, or the fragment set forth in SEQ ID NO:22, or the fragment set forth in SEQ ID NO:23, or the fragment set forth in SEQ ID NO:24, or the fragment set forth in SEQ ID NO:25, or the fragment set forth in SEQ ID NO:26, or the fragment set forth in SEQ ID NO:27, or the fragment set forth in SEQ ID NO:28, or the fragment set forth in SEQ ID NO:29, or the fragment set forth in SEQ ID NO:30, or the fragment set forth in SEQ ID NO: 31, or the fragment set forth in SEQ ID NO:32, or the fragment set forth in SEQ ID NO:33, or the fragment set forth in SEQ ID NO:34, or the fragment set forth in SEQ ID NO:37. The invention also relates to a variant of any of the above fragments or any other fragment described herein.

Preferably, the fragment is selected from the group of fragments set forth in SEQ ID NO:7-16 and 37, such as the fragment set forth in SEQ ID NO:7, or the fragment set forth in SEQ ID NO:8, or the fragment set forth in SEQ ID NO:9, or the fragment set forth in SEQ ID NO:10, or the fragment set forth in SEQ ID NO:11, or the fragment set forth in SEQ ID NO:12, or the fragment set forth in SEQ ID NO:13, or the fragment set forth in SEQ ID NO:14, or the fragment set forth in SEQ ID NO:15, or the fragment set forth in SEQ ID NO:16, or the fragment set forth in SEQ ID NO:37, or a variant of any of these fragments.

Compositions of the Invention

Compositions of the invention comprising one or more of polypeptides of the invention can be used in various methods and for various applications as described below. Having more than one polypeptide of the invention in such a composition can have important advantages. For instance, immunisation or vaccination may be more effective when several polypeptides or fragments are introduced at the same time.

Thus, in a main aspect the invention relates to a composition comprising one or more extracellular Aspergillus polypeptides or polypeptide fragments of the invention. Preferred compositions of the invention are ones that comprise one or more preferred polypeptides of the invention, i.e. the polypeptides described above. Thus, any preferred polypeptide of the invention can be used to generate a preferred composition of the invention. A preferred composition of the invention is a pharmaceutical composition comprising one or more polypeptide(s) and/or one or more polypeptide fragments of the invention and a pharmaceutically-acceptable carrier.

CssI Isopropylmalate Dehydrogenase B and GAPDH-B

Three extracellular polypeptides that were identified by the inventors are of particular interest, namely CssI, isopropylmalate dehydrogenase B, and GAPDH-B.

CssI

This document presents data indicating the first identifications of CssI, a novel cell-surface-exposed/secreted protein. This protein had previously been hypothesised based on the output of a gene prediction programme. However, the inventors' studies have confirmed the existence of this protein and have revealed it to be a conidial cell-wall-associated protein that is exposed on the surface while also being secreted/released into the surrounding milieu. The function of this protein is yet to be determined. However, its location within the diffusate is interesting in light of the documented abilities of diffusate to suppress the immune responses (Hobson R P (2000) Med. Mycol. 38, 133-141). Attempts to identify the protein(s) responsible for this suppressing activity have to date been unsuccessful. Without being limited to any specific theory, it is possible that CssI is responsible for these functions, but that they have not been attributed to it due to the basic difficulties in performing molecular biology studies in Aspergillus fumigatus. It is interesting to note that the protein displays homology to LANA, a transcriptional regulator of Herpes virus (see below under Examples). Again without limitation to a specific theory, the possibility exists that CssI possesses a similar function. If so, one could envisage it functioning as an extracellular sensor that transmits signals into the interior of the fungus. Alternatively, this protein may become active upon uptake into the host cell, where it utilises its transcriptional activities to interfere with host processes, to the benefit of the fungus. Other possible functions of this protein may include roles in adhesion, invasion, conidial cell-wall processing or enzymatic digestion of host proteins.

Isopropylmalate Dehydrogenase B

Isopropylmalate dehydrogenase B (IMDH B) is an enzyme involved in the biosynthesis of leucine. It has previously been found intracellularly in other microorganisms. The inventors have now identified this protein in cell surface fractions of A. fumigatus. The primary sequence of the enzyme does not reveal a traditional signal sequence and thus the question arises as to how the enzyme is transported to the cell surface. Without being limited to a particular theory, it is possible that the protein interacts with a heat shock protein and that the heat shock protein mediates translocation of IMDH B across the membrane. Similar mechanisms have been described for other proteins in Young et al. (2003) Cell 112:41-50.

GAPDH-B

The inventors have been the first to identify GAPDH-B, the polypeptide of SEQ ID NO:3. In one aspect, the invention relates to the sequence set forth in SEQ ID NO:3, and variants thereof. Furthermore, the invention relates to use of the polypeptide set forth in SEQ ID NO:3 in any of the methods or preferred methods of the invention.

GAPDHs are documented to function in glycolysis. Without being limited to a particular theory, the cell-surface localisation of GAPDH-B might suggest a role for this protein in the initiation of germination. It would seem logical to assume that dormant conidia are more prone to germination when environmental conditions become more favourable for growth and propagation of the species. One requirement for growth is a carbon source, e.g. glucose. However, the dormant conidia must have some way of detecting external environmental conditions while in its state of low metabolic activity. It is possible that the presence of glycolytic enzymes on the cell surface could result in the production of glucose by-products that may communicate to the cell that the external environment is of sufficient status to support propagation of the species. The protein may alternatively or additionally function in other processes such as adhesion, invasion, intracellular motility or intracellular survival. It is interesting to note that GAPDH proteins possess the capability to bind to cytoskeletal components (see e.g. Tisdale (2002) J. Biol. Chem. 277, 3334-3341). This feature may provide conidia with a mechanism by which it can traverse host cells in order to reach the basal membranes and cause invasive disease.

Production of Polypeptide and Fragments

The polypeptides and fragments of the invention can be produced synthetically by conventional techniques known in the art. Alternatively, they can be produced recombinantly in heterologous host cells. Thus, the invention also encompasses polynucleotide sequences encoding polypeptides and fragments of the invention, expression vectors comprising such polynucleotides, and host cells transformed or transfected with such polynucleotides or expression vectors. Non-exclusive examples of polynucleotides of the invention are the polynucleotides of SEQ ID NO:38 and SEQ ID NO:39. Suitable host cells can be mammalian cells, e.g. CHO, COS or HEK293 cells. Alternatively, insect cells, bacterial cells or fungal cells can be used. In preferred embodiments, yeast cells or cells from other Aspergillus species than A. fumigatus are used. Methods for heterologous expression of polynucleotide sequences in the cell types listed above and subsequent purification of the produced polypeptides are well-known to those skilled in the art.

Preferably, polypeptides, fragments and polynucleotides of the invention are isolated.

Vaccination

Exposure of a fungal polypeptide or a fragment thereof to the extracellular space often allows it to be detected by the immune system of a host organism. If such a polypeptide furthermore has a relatively high copy number, such as is the case for the extracellular polypeptides of this invention, such a polypeptide or a fragment thereof becomes particularly suitable as a target for antibodies.

In an important aspect, the invention relates to use of any one or more of the polypeptides, polynucleotides or compositions as defined herein for the manufacture of a medicament, preferably a vaccine. Such a medicament can preferably be used for prevention (i.e. prophylactic treatment) of Aspergillus infections in a mammal. In such use the polypeptide, polynucleotide or composition is used for active immunisation or vaccination. Accordingly, the invention also relates to a medicament for treating Aspergillus infections comprising a polypeptide, polynucleotide or composition of the invention as an active ingredient.

Thus, the invention relates to a vaccine comprising a pharmaceutically-acceptable carrier and
a polypeptide comprising a sequence selected from the group of extracellular Aspergillus fumigatus sequences of SEQ ID NO:1-6, or an antigenic fragment of any of said sequences, or
a polynucleotide comprising a sequence encoding said polypeptide or fragment.

Furthermore, the invention relates to a method of treatment comprising the step of administering to an individual a pharmaceutically effective amount of any of the polypeptides, polynucleotides or compositions of the invention. Preferably, the treatment generates a protective immune response. Preferably, the medicament is used for the treatment or prophylactic treatment of a human being. Preferred embodiments include the use of any of the polypeptides set forth in SEQ ID NO:1, 2, 3 or 36 or fragments of these polypeptides for said manufacture of said medicament or said method of treatment, preferably any of the preferred polypeptide fragments defined herein.

In preferred embodiments of this method, said polypeptide is selected from the group of SEQ ID NOs:1, 2, 3, 5, 6, and 36. In a more preferred embodiment, the polypeptide that is provided is CssI (SEQ ID NO:1) or a fragment thereof, preferably a fragment comprising one or more residues of the amino-acid sequences set forth in SEQ ID NO:7, 8, 17, 26, 28, 29 and/or 30, or a variant of said polypeptide. In another more preferred embodiment, the polypeptide that is provided is hydrophobin (SEQ ID NO:2) or a fragment thereof, preferably a fragment comprising one or more residues of the amino-acid sequences set forth in SEQ ID NO:9, 10, 18 and/or 19, or a variant of said polypeptide. In a further more preferred embodiment GAPDH-B (SEQ ID NO:3) or a fragment thereof, preferably a fragment comprising one or more residues of the amino-acid sequences set forth in SEQ ID NO:11, 12, 20, 21, 22, 31, 32 and/or 33, or a variant of GAPDH-3 or the fragment is provided. In a still further more preferred embodiment, the polypeptide that is provided is catalase A (SEQ ID NO:6) or a fragment thereof, preferably a fragment comprising one or more residues of the amino-acid sequence set forth in SEQ ID NO:34, or a variant of said polypeptide. In a further more preferred embodiment, the polypeptide that is provided is isopropylmalate dehydrogenase B (SEQ ID NO:36) or a variant or fragment thereof, preferably a fragment comprising one or more residues of the amino-acid sequence set forth in SEQ ID NO:37.

In other preferred embodiments, a fragment selected from the group of SEQ NO:7-34 and 37 is provided, preferably a fragment selected from the group of SEQ ID NO:7-16, such as the fragment set forth in SEQ ID NO:7, or the fragment set forth in SEQ ID NO:8, or the fragment set forth in SEQ ID NO:9, or the fragment set forth in SEQ ID NO:10, or the fragment set forth in SEQ ID NO:11, or the fragment set forth in SEQ ID NO:12, or the fragment set forth in SEQ ID NO:13, or the fragment set forth in SEQ ID NO:14, or the fragment set forth in SEQ ID NO:15, or the fragment set forth in SEQ ID NO:16.

Active immunisation or vaccination may be done in different ways, such as raising anti-protein antibodies indirectly using DNA immunisation techniques or directly using the polypeptide or a fragment thereof. The polypeptide may be administrated to said mammal more than once, such as twice, for example 3 times, such as 3 to 5 times, for example 5 to 10 times, such as 10 to 20 times, for example 20 to 50 times, such as more than 50 times. It is also possible that different polypeptides or fragments are administered to the same mammal, either simultaneously of sequentially in any order. Administration may be done by any suitable method, for example parenterally, orally or topically. Preferably, however it is administered by injection, for example intramuscular, intradermal, intravenous or subcutaneous injection, more preferably by subcutaneous or intravenous injection.

Methods for determining suitable protocols for active immunisation, such as determining dosage, use of adjuvants and/or pharmaceutically acceptable carriers are known to those skilled in the art. Various adjuvants can be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Antibodies

In another main aspect, the invention relates to isolated antibodies capable of binding an extracellular Aspergillus fumigatus polypeptide selected from the group consisting of isopropylmalate dehydrogenase B (SEQ ID NO:36), CssI (SEQ ID NO:1), hydrophobin (SEQ ID NO:2), GAPDH-B (SEQ ID NO:3), and catalase A (SEQ ID NO:6).

In preferred embodiments, the antibody is capable of binding a polypeptide selected from the group consisting of isopropylmalate dehydrogenase B (SEQ ID NO:36), CssI (SEQ ID NO:1) and catalase A (SEQ ID NO:6). More preferably, the antibody is capable of binding a polypeptide selected from the group consisting of isopropylmalate dehydrogenase B (SEQ ID NO:36) and CssI (SEQ ID NO:1). Most preferably, the antibody is capable of binding isopropylmalate dehydrogenase B (SEQ ID NO:36).

Preferred affinities of the binding of the antibody to the target polypeptide include those with a dissociation constant or Kd of less than 5×10⁻⁴M, such as less than 10⁻⁴M, e.g. less than 5×10⁻⁵M, such as less than 10⁻⁵M, e.g. less than 5×10⁻⁶M, such as less than 10⁻⁶M, e.g. less than 5×10⁻⁷M, such as less than 10⁻⁷M, e.g. less than 5×10⁻⁸M, such as less than 10⁻⁸ M, e.g. less than 5×10⁻⁹M, such as less than 10⁻⁹M, e.g. less than 5×10⁻¹⁰M, such as less than 10⁻¹¹M, e.g. less than 5×10⁻¹¹M, such as less than 10⁻¹¹M, e.g. less than 5×10⁻¹²M, such as less than 10⁻¹² M, e.g. less than 5×10⁻¹³ M, such as less than 10⁻¹³ M, e.g. less than 5×10⁻¹⁴M, such as less than 10⁻¹⁴M, e.g. less than 5×10⁻¹⁵M, or less than 10⁻¹⁵M. Binding constants can be determined using methods well-known in the art, such as ELISA (e.g. as described in Orosz and Ovadi (2002) J. Immunol. Methods 270:155-162) or surface plasmon resonance analysis.

In preferred embodiments, the antibody of the invention is capable of binding an intact Aspergillus fumigatus cell, i.e. capable of binding a living or a dead Aspergillus cell which has maintained its structural integrity, preferably a cell that has maintained the integrity of the plasma membrane (i.e. wherein the plasma membrane is not permeabilised). Binding of antibodies to intact cells can e.g. be tested as described in the Examples herein.

In further preferred embodiments, the antibody of the invention, or at least an Fab fragment thereof, is capable of reducing the adhesion of Aspergillus fumigatus conidia to lung epithelia in an in vitro assay set-up as described herein in the Examples, preferably reducing said adhesion with at least 20%, such as at least 40%, e.g. at least 60% or at least 80%.

Furthermore, it is preferred that the antibody of the invention, or at least an Fab fragment thereof, is capable of reducing the germination of Aspergillus fumigatus conidia in an in vitro assay set-up as described herein in the Examples, preferably reducing said adhesion with at least 20%, such as at least 40%, e.g. at least 60% or at least 80%.

The antibodies of the invention are capable of specifically recognising and binding an Aspergillus fumigatus target polypeptide selected from the group of SEQ ID NO:1, SEQ ID NO:2, SEQ ID NO:3, SEQ ID NO:6 and SEQ ID NO:36. ‘specifically’ is, in this content, not intended to mean absolute specificity. Thus, ‘species-specific’ is used when it is intended to indicate that the antibody cannot bind to homologous polypeptides from other fungi.

In some embodiments, the antibody of the invention is, in addition to being capable of binding an Aspergillus fumigatus polypeptide, capable of binding a homologous polypeptide from another fungus. E.g. in these embodiments, the antibody of the invention is further capable of binding a homologous polypeptide, wherein the homologous polypeptide has a sequence identity of 39% or more, such as 42% or more, e.g. 48% or more, such as 68% or more, e.g. 80% or more, such as 90% or more, to a polypeptide selected from the group consisting of isopropylmalate dehydrogenase B (SEQ ID NO:36), CssI (SEQ ID NO:1), hydrophobin (SEQ ID NO:2), GAPDH-B (SEQ ID NO:3), and catalase A (SEQ ID NO:6). Preferred are antibodies that are capable of binding a homologous polypeptide originating from one of the following species:
an Aspergillus species, such as Aspergillus fumigatus, Aspergillus nidulans, Aspergillus niger, or Aspergillus oryzea,
Neurospora crassa,
Saccharomyces cerevisiae,
a Candida species such as Candida albicans,
a Coccidioides species, such as Coccidioides posadasii, or Coccidioides immitis,
a Cryptococcus species, such as Cryptococcus neoformans var. neoformans,
a Fusarium species,
a Pneumocystis species,
a Penicillium species,
Histoplasma capsulatum.
   More preferably, the homologous polypeptide originates from
an Aspergillus species, such as Aspergillus fumigatus, Aspergillus nidulans, Aspergillus niger or Aspergillus oryzea,
Candida albicans,
Coccidioides posadasii, or
Cryptococcus neoformans var. neoformans.

In one specific embodiment, the antibody of the invention further recognises a homologous polypeptide which also originates from Aspergillus fumigatus, such as the polypeptide of SEQ ID NO:41.

In preferred examples of the type of embodiments described above, said homologous polypeptide is also extracellular. Thus, the antibody capable of binding both an Aspergillus fumigatus polypeptide as well as a homologous polypeptide, will be capable of binding an intact cell of any one or more of the species from which the homologous polypeptide originates, i.e. one of
an Aspergillus species other than Aspergillus fumigatus, such as Aspergillus nidulans, Aspergillus niger, or Aspergillus oryzea,
Neurospora crassa,
Saccharomyces cerevisiae,
a Candida species such as Candida albicans,
a Coccidioides species, such as Coccidioides posadasii, or Coccidioides immitis,
a Cryptococcus species, such as Cryptococcus neoformans var. neoformans,
a Fusarium species,
a Pneumocystis species,
a Penicillium species, and
Histoplasma capsulatum.

Binding of the antibodies of the invention to homologous polypeptides and/or other intact cells of other fungi can be tested by the method described herein in the Examples or by other standard methods known in the art.

In preferred embodiment, the antibody of the invention is capable of binding one or more amino acid residues comprised within a region of SEQ ID NO:36 that has significant identity to homologous polypeptides from other fungi, such as regions of SEQ ID NO:36 that have identity with a homologous polypeptide of 4, 5, 6, 7, 8 or more consecutive amino acids. Most preferably, antibody of the invention recognises an epitope which is entirely consisting of residues comprised within one of these regions of SEQ ID NO:36. In particular the following regions are preferred: Ser67-Leu71, Ala74-Trp80, Ser191-Arg205, Leu268-Leu273, His292-Pro296, Glu355-Ile360, Asp193-Glu209, Asp193-Ala199, Ile15-Val19, Val75-Trp80, and Pro11-Glu18. Further preferred are antibodies that bind to an epitope of SEQ ID NO:36 which comprises one or more residues of SEQ ID NO:37. Methods for epitope mapping are well known in the art.

In a different set of embodiments, the antibody of the invention is not capable of binding intact cells from one or more another fungi. For instance, in one such embodiment, the antibody is not capable of binding an intact cell of any of
Neurospora crassa,
Saccharomyces cerevisiae,
Candida albicans,
Coccidioides posadasii, or Coccidioides immitis,
Cryptococcus neoformans var. neoformans, or
Histoplasma capsulatum.

More preferably, the antibody of the invention is not capable of binding an intact cell of any of
Aspergillus nidulans
Aspergillus niger
Aspergillus oryzea,
Neurospora crassa,
Saccharomyces cerevisiae,
Candida albicans,
Coccidioides posadasii, or Coccidioides immitis,
Cryptococcus neoformans var. neoformans, or
Histoplasma capsulatum.

In one specific embodiment, the antibody of the invention is species-specific, i.e. not capable of binding homologous polypeptides or intact cells from other fungi than Aspergillus fumigatus.

The invention also relates to pharmaceutical compositions comprising an antibody of the invention and a pharmaceutically-acceptable carrier.

Raising Antibodies and Functional Equivalents

In another aspect, the invention relates to a method for raising specific antibodies to a polypeptide selected from the group of polypeptides set forth in SEQ ID NOs: 1-6 and 36 in a non-human animal comprising the steps of

a. providing a polypeptide selected from the group of polypeptides set forth in SEQ ID NOs: 1-6 and 36 or a polypeptide selected from the group of the polypeptide fragments as defined in the present application, or a cell expressing any of these polypeptides,

b. introducing a composition comprising said polypeptide or said cell into said animal,

c. raising antibodies in said animal, and

d. isolating and optionally purifying the antibodies.

In one embodiment of the above method, the polypeptide that is provided is CssI (SEQ ID NO:1) or a fragment thereof, or a variant of said polypeptide. In another embodiment of the above method, the polypeptide that is provided is hydrophobin (SEQ ID NO:2) or a fragment thereof, or a variant of said polypeptide. In yet another embodiment of the above method, the polypeptide that is provided is GAPDH-B (SEQ ID NO:3) or a fragment thereof, or a variant of said polypeptide. In a yet further embodiment of the above method, wherein the polypeptide that is provided is catalase A (SEQ ID NO:6) or a fragment thereof, or a variant of said polypeptide. And in an even further embodiment of the above method, the polypeptide that is provided is isopropylmalate dehydrogenase B (SEQ ID NO:36) or a fragment thereof, or a variant of said polypeptide.

Antibodies include polyclonal antibodies, monoclonal antibodies, human, humanised or chimeric antibodies, single-chain antibodies, and also Fab fragments, F(ab′)₂ fragments, fragments produced by a Fab expression library, anti-idiotypic antibodies, hybrids comprising antibody fragments, and epitope-binding fragments of any of the these. The term also includes mixtures of monoclonal antibodies.

In some embodiments, the antibody of the invention is polyclonal. Polyclonal antibodies are heterogeneous populations of antibody molecules derived from the sera of animals immunised with an antigen, such as one of the extracellular polypeptides identified by the inventors, or a fragment, epitope or variant thereof. For the production of polyclonal antibodies, host animals can be immunised by injection with the polypeptide supplemented with adjuvants. The antibody titer in the immunised animal can be monitored over time by standard techniques, such as ELISA using immobilised polypeptide. If desired, the antibody molecules can be isolated from the animal, for instance from the blood, and further purified by well-known techniques, such as protein-A chromatography, to obtain the IgG fraction. Thus, in a preferred embodiment, the above described method for generating an immune response comprises a step d. of isolating and purifying antibodies generated in said immune response.

In other embodiments, the antibody of the invention is monoclonal. Monoclonal antibodies, which are homogeneous populations of antibodies to a particular epitope, can be obtained by any technique which provides for the production of antibody molecules by continuous cell-lines in culture. These include, but are not limited to the hybridoma technique of Kohler and Milstein ((1975) Nature 256, 495-497; and U.S. Pat. No. 4,376,110), the human B-cell hybridoma technique (Kosbor et al., 1983, Immunology Today 4, 72; Cole et al., 1983, Proc. Natl. Acad. Sci. USA 80, 2026-2030), and the EBV-hybridoma technique (Cole et al., 1985, Monoclonal Antibodies And Cancer Therapy, Alan R. Liss, Inc., pp. 77-96). Such antibodies can be of any immunoglobulin class including IgG, IgM, IgE, IgA, IgD and any subclass thereof. A preferred class is IgG1. The hybridoma producing the monoclonal antibody of this invention can be cultivated in vitro or in vivo.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage-display library) with the polypeptide of interest or a fragment thereof. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; WO 92/18619; WO 91/17271; WO 92/20791; WO 92/15679; WO 93/01288; WO 92/01047; WO 92/09690; WO 90/02809; Fuchs et al. (1991) Bio/Technology 9: 1370-1372; Hay et al. (1992) Hum. Antibod. Hybridomas 3, 81-85; Huse et al. (1989) Science 246, 1275-1281; and Griffiths et al. (1993) EMBO J. 12, 725-734.

Additionally, recombinant antibodies, such as chimeric and humanised monoclonal antibodies comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., U.S. Pat. No. 4,816,567; and U.S. Pat. No. 4,816,397, which are incorporated herein by reference in their entirety.) Humanised antibodies are antibody molecules from non-human species having one or more complementarity-determining regions from the non-human species and a framework region from a human immunoglobulin molecule. (See, e.g. U.S. Pat. No. 5,585,089, which is incorporated herein by reference in its entirety.) Such chimeric and humanised monoclonal antibodies can be produced by recombinant DNA techniques known in the art, for example using methods described in WO 87/02671; European Patent Application 184,187; European Patent Application 171,496; European Patent Application 173,494; WO 86/01533; U.S. Pat. No. 4,816,567; European Patent Application 125,023. Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyen et al. (1988) Science 239:1534-1536; Beidler et al. (1988) J. Immunol. 141:4053-4060; and Westin Kwon et al. (2002) Clin. Diagn. Lab. Immunol. 9, 201-204.

In a highly preferred embodiment, the antibody of the invention is a human antibody. Completely human antibodies are particularly desirable for therapeutic treatment of human patients. Such antibodies can be produced using transgenic mice which are incapable of expressing endogenous immunoglobulin heavy and kappa light chains genes, but which can express human heavy and light chain genes. The transgenic mice are immunised in the normal fashion with a selected antigen, e.g., all or a fragment of a polypeptide of the invention. onoclonal antibodies directed against the antigen can e.g. be obtained using conventional hybridoma technology. The human immunoglobulin transgenes harboured by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995) Int. Rev. Immunol. 13: 65-93). For a detailed discussion of this technology for producing human antibodies and human monoclonal antibodies and protocols for producing such antibodies, see, e.g. WO 02/43478; U.S. Pat. No. 5,625,126; U.S. Pat. No. 5,633,425; U.S. Pat. No. 5,569,825; U.S. Pat. No. 5,661,016; and U.S. Pat. No. 5,545,806. Completely human antibodies which recognise a selected epitope can be generated using a technique referred to as “guided selection”. In this approach a selected non-human monoclonal antibody, e.g. a mouse antibody, is used to guide the selection of a completely human antibody recognising the same epitope (see Jespers et al. (1994) Bio/Technology 12, 899-903).

Highly suitable methods for the production of human monoclonal antibodies have been described in WO 04/035607 (Genmab) and WO 04/043989 (Medarex). Further similar methods have been described in WO 03/017935 (Genmab), WO 02/100348 (Genmab), WO 02/064634 (Medarex) and WO 03/040169 (Medarex).

Antibody fragments which recognise specific epitopes can be generated by known techniques. For example, such fragments include but are not limited to: F(ab′)₂ fragments which can be produced by pepsin digestion of the antibody molecule and Fab fragments which can be generated by reducing the disulfide bridges of the F(ab′)₂ fragments. Alternatively, Fab expression libraries can be constructed (Huse et al. (1989) Science 246, 1275-1281) to allow rapid and easy identification of monoclonal Fab fragments with the desired specificity.

Antibodies of the invention also include bispecific antibodies having two binding specificities, of which at least one is a specificity for a polypeptide selected from the group of SEQ ID NO:1-6 and 36, preferably selected from the group of SEQ ID NOs:1-4 and 36.

In preferred embodiments, the antibody of the invention is purified.

Antibody Treatment

Antibodies can be used for passive immunisation of mammals, preferably human beings, more preferably immunocompromised patients. A treatment with antibodies can be done to cure or to prevent Aspergillus infections. Thus, the invention relates to use of an antibody as defined herein for the manufacture of a medicament, preferably a medicament for the treatment of fungal infections or the prophylactic treatment (prevention) of fungal infections, preferably Aspergillus infections, such as Aspergillus fumigatus infections. Examples of fungal infections are invasive aspergillosis, aspergilloma, and allergic aspergillosis, such as allergic bronchopulmonary aspergillosis.

Formulated in another way, the invention relates to an antibody as defined herein or a composition as defined herein for use as a medicament. The invention also relates to a method of treatment comprising the step of administering to an individual a pharmaceutically-effective amount of an antibody of the invention as defined herein, and to a medicament for treating Aspergillus infections comprising an antibody of the invention as an active ingredient.

Antibodies of the invention may be mechanistically divided into the following preferred groups:

1. Function-inhibiting antibodies that work as an antifungal (i.e. affect the viability of the fungus, including both fungicidal and fungistatic effects). Such antibodies should be effective regardless of the immune status of the patient. This category of antibodies includes pathogenesis-inhibiting antibodies, which block a protein required for disease (Adhesion/Invasion), and growth-inhibiting antibodies, which block a protein required for germination and/or sporulation.

2. Opsonising antibodies that are designed to enhance phagocytic killing. Effectiveness of such antibodies may depend on the immune status of the patient, but it is very well possible that they will enhance phagocytic killing even in compromised patients. Opsonising antibodies also comprise antibodies which enhance clearance by the immune system via complement and phagocytosis.

3. Antibodies conjugated to a therapeutic moiety such as a toxin or fungicidal agent, e.g. ricin or radioisotopes, directed against fungal surface components. Techniques for conjugating a therapeutic moiety to antibodies are well known, see, e.g. Thorpe et al. (1982) Immunol. Rev. 62, 119-158. These antibodies should also be effective regardless of the immune status of the patient.

Validation of targets and antibodies can be done by the following methods known in the art:
Invasion assays—test whether invasion of Aspergillus into lung cells is prevented
Adhesion assays—test whether adhesion to and colonisation of lung cells is prevented
Germination assays—test whether growth and germination is prevented
Opsonisation assay—test whether function is inhibited, and clearance is eased
Aggregation assays—test whether clumping is prevented, and whether clearance is eased
Invasive disease animal models—test whether disease is prevented

In another aspect, the present invention provides a composition, e.g., a pharmaceutical composition, comprising an antibody, e.g. a human monoclonal antibody of the present invention. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

The pharmaceutical composition may be administered by any suitable route and mode. As will be appreciated by the skilled artisan, the route and/or mode of administration will vary depending upon the desired results. The pharmaceutical compositions of the present invention include those suitable for oral, nasal, topical (including buccal and sublingual), rectal, vaginal and/or parenteral administration. Formulations of the present invention which are suitable for vaginal administration include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants.

The pharmaceutical composition is preferably administered parenterally. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In one embodiment the pharmaceutical composition is administered by intravenous or subcutaneous injection or infusion. In one embodiment the antibodies of the invention are administered in crystalline form by subcutaneous injection, cf. Yang et al. (2003) PNAS, 100(12):6934-6939.

Regardless of the route of administration selected, the compounds of the present invention, which may be used in the form of a pharmaceutically acceptable salt or in a suitable hydrated form, and/or the pharmaceutical compositions of the present invention, are formulated into pharmaceutically acceptable dosage forms by conventional methods known to those of skill in the art.

As used herein, “pharmaceutically acceptable carrier” includes any and all solvents, dispersion media, coatings, antibacterial and antifungal agents, isotonicity agents, antioxidants and absorption delaying agents, and the like that are physiologically compatible. Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Preferably, the carrier is suitable for parenteral administration, e.g. intravenous or subcutaneous injection or infusion. Pharmaceutical compositions typically must be sterile and stable under the conditions of manufacture and storage. The composition can be formulated as a solution, microemulsion, liposome, or other ordered structure suitable to high drug concentration. Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The pharmaceutical compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol, sorbic acid, and the like. It may also be desirable to include isotonicity agents, such as sugars, polyalcohols such as mannitol, sorbitol, glycerol or sodium chloride in the compositions. Pharmaceutically-acceptable antioxidants may also be included, for example (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

If appropriate, the antibody may be used in a suitable hydrated form or in the form of a pharmaceutically acceptable salt. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Depending on the route of administration, the active compound, i.e., antibody, and bispecific/multispecific molecule, may be coated in a material to protect the compound from the action of acids and other natural conditions that may inactivate the compound. For example, the compound may be administered to a subject in an appropriate carrier, for example, liposomes. Liposomes include water-in-oil-in-water CGF emulsions as well as conventional liposomes (Strejan et al. (1984) J. Neuroimmunol. 7:27). The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such formulations are generally known to those skilled in the art. See, e.g., Sustained and Controlled Release Drug Delivery Systems, J. R. Robinson, ed., Marcel Dekker, Inc., New York, 1978.

The pharmaceutical compositions can be administered with medical devices known in the art. For example, in a preferred embodiment, a therapeutic composition of the invention can be administered with a needleless hypodermic injection device, such as the devices disclosed in U.S. Pat. No. 5,399,163, U.S. Pat. No. 5,383,851, U.S. Pat. No. 5,312,335, U.S. Pat. No. 5,064,413, U.S. Pat. No. 4,941,880, U.S. Pat. No. 4,790,824, or U.S. Pat. No. 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. No. 4,522,811; U.S. Pat. No. 5,374,548; and U.S. Pat. No. 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds of the invention are formulated in liposomes; in a more preferred embodiment, the liposomes include a targeting moiety. In a most preferred embodiment, the therapeutic compounds in the liposomes are delivered by bolus injection to a site proximal to the desired area, e.g., the site of infection. The composition must be fluid to the extent that easy syringability exists. It must be stable under the conditions of manufacture and storage and must be preserved against the contaminating action of microorganisms such as bacteria and fungi.

In a further embodiment, the antibodies of the invention can be formulated to prevent or reduce their transport across the placenta. This can be done by methods known in the art, e.g., by PEGylation of the antibodies or by use of F(ab′)2 fragments. Further references can be made to “Cunningham-Rundles C, Zhuo Z, Griffith B, Keenan J. (1992) Biological activities of polyethylene-glycol immunoglobulin conjugates. Resistance to enzymatic degradation. J Immunol Methods. 152:177-190; and to “Landor M. (1995) Maternal-fetal transfer of immunoglobulins, Ann Allergy Asthma Immunol 74:279-283.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound which is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic composition may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

In one embodiment, the antibodies according to the invention can be administered by infusion in a weekly or daily dosage of from 10 to 500 mg/m2, such as of from 200 to 400 mg/m2. Such administration can be repeated, e.g., 1 to 8 times, such as 3 to 5 times. The administration may be performed by continuous infusion over a period of from 2 to 24 hours, such as of from 2 to 12 hours. In another embodiment, the human monoclonal antibodies can be administered by slow continuous infusion over a long period, such as more than 24 hours, in order to reduce toxic side effects.

In still another embodiment the human monoclonal antibodies can be administered in a weekly dosage of from 250 mg to 2000 mg, such as for example 300 mg, 500 mg, 700 mg, 1000 mg, 1500 mg or 2000 mg, for up to 8 times, such as from 4 to 6 times. The administration may be performed by continuous infusion over a period of from 2 to 24 hours, such as of from 2 to 12 hours. Such regimen may be repeated one or more times as necessary, for example, after 6 months or 12 months. In yet another embodiment, the human monoclonal antibodies can be administered by maintenance therapy, such as, e.g., once a week for a period of 6 months or more.

Combination Treatment

The pharmaceutical composition of the invention may contain one or a combination of antibodies of the invention. Thus, in a further embodiment, the pharmaceutical compositions include a combination of multiple (e.g., two or more) isolated antibodies of the invention which act by different mechanisms.

Treatment of antifungal infections as defined herein, for example passive immunisation with antibodies specifically recognising and binding the extracellular Aspergillus polypeptides as defined herein, can also be combined with any other type of therapy, in particular other antifungal therapy. Thus, an antibody with or without a therapeutic moiety conjugated to it can be used as a therapeutic that is administered alone or in combination with antifungal chemotherapeutics or other therapeutic agents. For instance, treatment with an antibody as defined herein can be combined with treatment with antifungal compounds, such as azoles (e.g. fluconazole, itraconazole, ketoconazole, miconazole), amphotericin B, flucytosine, or echinocandins, such as Caspofungin. Alternatively, or in addition, treatment with antibodies as defined herein can also be combined with treatment with other antifungal antibodies, for instance antibodies directed against HSP90, such as Mycograb (Matthews et al. (2003) Antimicr. Agents and Chemotherapy 47:2208-2216).

Combination therapy can in some circumstances be more effective than single component therapy. Combination therapy can be particularly useful for patients that suffer from infection with multiple fungal species, for example patients having both a Candida and an Aspergillus infections.

Binding Partners and Inhibitors of Extracellular Polypeptides

In addition to antibodies, it is of interest to identify other types of binding partners to extracellular polypeptides. Extracellular polypeptides of a pathogenic fungus often interact with the host organism. Any type of binding partner of an extracellular polypeptide may interfere with host-pathogen interaction. Binding partners may thus antagonise the pathogenicity of the fungus.

Identification of binding partners of the extracellular polypeptides set forth in SEQ ID NO:1-6 and 36, or fragments thereof, is another main aspect of this invention. This may be done using biochemical or cell-based methods.

Biochemical Methods

In a main aspect, the invention relates to a method for identifying a binding partner of a polypeptide of the invention and/or a polypeptide selected from the group of SEQ ID NOs:1-6 and 36, comprising the steps of
a. providing a polypeptide of the invention as defined herein or a polypeptide selected from the group of SEQ ID NOs:1-6 and 36,
b. contacting said polypeptide with a putative binding partner, and
c. determining whether said putative binding partner is capable of binding to said polypeptide.

Thus, in one embodiment of the above method, the polypeptide that is provided is CssI (SEQ ID NO:1) or a fragment thereof, or a variant of said polypeptide. In another embodiment, the polypeptide that is provided is hydrophobin (SEQ ID NO:2) or a fragment thereof, or a variant of said polypeptide. In another embodiment, the polypeptide that is provided is GAPDH-B (SEQ ID NO:3) or a fragment thereof, or a variant of said polypeptide. In a yet other embodiment, the polypeptide that is provided is catalase B (SEQ ID NO:5) or a fragment thereof, or a variant of said polypeptide. In a further embodiment, wherein the polypeptide that is provided is catalase A (SEQ ID NO:6) or a fragment thereof, or a variant of said polypeptide. In an even further embodiment, wherein the polypeptide that is provided is isopropylmalate dehydrogenase B (SEQ ID NO:36) or a fragment thereof, or a variant of said polypeptide.

In a preferred embodiment of this method of the invention, said polypeptide is selected from the group of SEQ ID NOs:1, 2, 3, 5, 6, and 36 such as the polypeptide set forth in SEQ ID NO:1, or the polypeptide set forth in SEQ ID NO:2, or polypeptide set forth in SEQ ID NO:3, or the polypeptide set forth in SEQ ID NO:5, or the polypeptide set forth in SEQ ID NO:6 or the polypeptide set forth in SEQ ID NO:36. In other preferred embodiments, an exposed domain, an epitope or a fragment of one of the polypeptides set forth in SEQ ID NOs:1-6 and 36 comprising one or more amino-acid residues of the sequences set forth in SEQ ID NO: 7-34 and 37 is provided in step a. In further preferred embodiments, a fragment selected from the group of SEQ NO:7-34 and 37 is provided, preferably a fragment selected from the group of SEQ ID NO:7-27 and 37, or a variant or fragment of any of the amino-acid sequences set forth in SEQ ID NO:7-27 and 37.

In preferred embodiments of this method, the polypeptide or fragment thereof is provided immobilised on a solid support, such as e.g. a column or microtiter plate, and, after the contacting step, it is determined whether or not the putative binding partner has bound to the solid support. Immobilisation of the polypeptide or fragment thereof may be through direct binding to the solid support, or through indirect binding e.g. using a specific antibody. In preferred embodiments, a washing step is performed between the contacting step and the determination step, in order to improve the specificity of detection. In further preferred embodiments, the putative binding partner is labelled. The putative partner may be labelled before the contacting takes place. Alternatively, labelling may also be performed after the contacting step. Furthermore, in some embodiments of this method, immobilisation may be performed after the polypeptide or fragment thereof has been bound to the binding partner. In preferred embodiments, the method is repeated for a plurality of putative binding partners. Putative binding partners include host-derived molecules.

Alternatively, a binding partner of a polypeptide of the invention or of a polypeptide selected from the group of SEQ ID NO:1, 2, 4, 5, 6 and 36 may be identified as follows: purified host membranes are electrophoretically separated, blotted over to a membrane and incubated with the polypeptide of interest or fragment thereof. Binding can then be detected using antibodies specific for the polypeptide of interest or fragment thereof. The host binding partner to which the polypeptide or fragment thereof has bound can subsequently be identified by elution from the blot and subsequent analysis by mass spectrometry, or by any other technique known in the art.

If the binding partner of an extracellular polypeptide of a pathogenic organism is a host-derived molecule, then such an interaction between the extracellular polypeptide and the host may be important for the virulence of the fungus. Compounds that interfere with the interaction of the extracellular polypeptide and the host binding partner may thus be suitable for prevention or treatment of fungal infections. Accordingly, another method of the invention relates to a method of identifying an inhibitor of the interaction of an extracellular Aspergillus polypeptide selected from the group of SEQ ID NO:1-6 and 36 or fragment thereof with a host-derived binding partner comprising the steps of:
a. providing a polypeptide selected from the group of SEQ ID NO:1-6 and 36, or a fragment thereof,
b. providing a host-derived binding partner of said polypeptide
c. contacting said polypeptide with said host-derived binding partner in the absence of a putative inhibitor of said interaction
d. contacting said polypeptide with said host-derived binding partner in the presence of said putative inhibitor
e. determining whether the strength of the binding of said polypeptide to said host-derived binding partner resulting from step d. is reduced as compared to that resulting from step c.

In some embodiments, step c. and d. may be performed in two different sample compartments. In other embodiments, step d. may be performed by adding the putative inhibitor to the mixture of step c. In preferred embodiments, a fragment selected from the group of SEQ ID NO:7-34 and 37 is provided in step a. In other preferred embodiments, the polypeptide of SEQ ID NO:1, 2, 3, 5, 6, or 36 is provided. In further preferred embodiments, the method is repeated for a plurality of putative inhibitors. Of further particular interest are binding partners that inhibit an activity of an extracellular polypeptide. Such activity may be enzymatic activity, transport activity, or any type of other biochemical or cellular activity, preferably enzymatic activity. Inhibitors of IMDH B, GAPDH, enolase or catalase may be screened for using known biochemical assays of the enzymes, such as the catalase assay kit of CALBIOCHEM, cat. no. 219263, and e.g. the assays described in Pirrung et al. (1996) J Org Chem 61, 4527-4531; Bartolini et al. (2003) J. Chromatogr. 987, 331-340; Lal et al.(1991) Plant Mol. Biol. 16, 787-795; Machida et al. (1996) Biosci Biotechnol Biochem 60, 161-163; and Maitra and Lobo (1971) J Biol Chem 246, 475-88.

Cell-based Methods

Reducing the level of an extracellular polypeptide, by deletion or disruption of the structural gene for it or by down-regulating gene expression (see below), may affect a fungal cell. The cell may become more sensitive to cytotoxic compounds. Especially for extracellular polypeptides, a reduction of their level may affect the function of the cell's exterior parts, such as the plasma membrane or cell wall, in preventing compounds of entering the cell. Thus, reduction of the level of an extracellular polypeptide can make a cell more ‘permeable’ for various compounds.

An aspect of the present invention relates to a method for identifying a compound with anti-Aspergillus fumigatus activity comprising the steps of

a. providing a sensitised cell which has a reduced level of a polypeptide selected from the group of SEQ ID NO:1-6 and 36, and

b. determining the sensitivity of said cell to a putative inhibitor, for instance by a growth assay.

In a preferred embodiment, a sensitised cell which has a reduced level of a polypeptide selected from the group of SEQ ID NO:1, 2, 3, 5, 6, and 36 is provided in step a. In an even more preferred embodiment of the method, a sensitised cell which has a reduced level of CssI (SEQ ID NO:1), hydrophobin (SEQ ID NO:2), GAPDH (SEQ ID NO:3), catalase A (SEQ ID NO:6), or isopropylmalate dehydrogenase B (SEQ ID NO:36) is provided.

The rationale behind this approach is that a cell with a lower level of the extracellular polypeptide will exhibit increased sensitivity to cytotoxic compounds, allowing identification of antifungal compounds with low potency that are missed when using wild-type cells for the assay. Compounds identified by this method will be often need to be modified in order to improve potency. This can be done by chemical modification. In preferred embodiments, the method is repeated for a plurality of putative binding partners.

Inhibition of the activity of an extracellular polypeptide may affect the viability (i.e. survival, growth and/or proliferation) of the fungus. Of particular interest is inhibition of extracellular polypeptides that are essential for viability of A. fumigatus. Essentiality of an Aspergillus gene may be investigated e.g. using regulatable expression as described in WO 02/086090. Inhibitors of essential extracellular polypeptides may not need to enter the fungal cell to be able to affect its viability. Thus, generally fewer requirements are posed on the structure of an inhibitor of essential extracellular target polypeptide than on an inhibitor of an intracellular target, to be effective as an antifungal agent.

Thus, the invention relates to a method for identifying an inhibitor of an extracellular Aspergillus fumigatus polypeptide selected from the group of SEQ ID NO:1-6 and 36 comprising the steps of:

a. providing two cells which differ in the level of a polypeptide selected from the group of SEQ ID NO:1-6 and 36,

b. determining the sensitivity of said cells to a putative inhibitor, for instance by a growth assay, and

c. determining whether said two cells are differently affected by the presence of said putative inhibitor.

The rationale behind this approach is that the viability of a cell with a lower activity of the essential polypeptide will be more affected by an inhibitor of the polypeptide than the viability of the cell with a higher level. If the two cells are differently affected, this is an indication that the inhibitor acts on the target or in the same biochemical pathway. In a preferred embodiment of the method, said polypeptide is CssI (SEQ ID NO:1), GAPDH (SEQ ID NO:3), catalase A (SEQ ID NO:6), or isopropylmalate dehydrogenase B (SEQ ID NO:36).

In some embodiments of the method, the two cells with different activity of the polypeptide of interest are a wild-type cell (or other cell with wild-type activity of the gene of interest) and a sensitised cell with a reduced activity of the polypeptide of interest. In some embodiments, the different or reduced level in the sensitised cell can be a different or reduced expression level of the gene of interest (resulting in a different or reduced copy number of the polypeptide). This can be accomplished by putting the gene under control of a regulatable promoter or by regulatable expression of an antisense RNA which inhibits translation of an mRNA encoding the essential polypeptide. In other embodiments, the different or reduced activity can be a different or reduced activity of the polypeptide of interest, e.g. due to a mutation, such as a temperature-sensitive mutation. In preferred embodiments, the method is repeated for a plurality of putative binding partners.

Suitable ways of generating sensitised cells and of using these in screening for inhibitors have been described in WO 02/086090. Sensitised cells may be obtained by growing a conditional-expression A. fumigatus mutant strain in the presence of a concentration of inducer or repressor which provides a level of a gene product required for fungal viability such that the presence or absence of its function becomes a rate-determining step for viability. A number of suitable regulatable promoters for constructing such conditional-expression mutants of Aspergillus is described in WO 02/086090, page 76, line 34 through page 85, line 4. For example, if the regulatable promoter is repressed by tetracycline, the conditional-expression Aspergillus fumigatus mutant strain may be grown in the presence of partially repressing concentrations of tetracyline. The sub-lethal concentration of inducer or repressor may be any concentration consistent with the intended use of the assay. For example, the sub-lethal concentration of the inducer or repressor may be such that growth inhibition is at least about 10%, such as at least about 25%, e.g. at least about 50%, such as at least about 75%, e.g. at least 90%, such as at least 95%.

Similarly, the virulence or pathogenicity of cells exposed to a candidate compound which express a rate-limiting amount of a gene product required for virulence or pathogenicity may be compared to the virulence or pathogenicity of cells exposed to the candidate compound in which the level of expression of the gene product required for virulence or pathogenicity is not rate-limiting. In such methods, test animals are challenged with the conditional-expression A. fumigatus mutant strain and fed a diet containing the desired amount of tetracycline and the candidate compound. Thus, the conditional-expression mutant strain infecting the test animals expresses a rate limiting amount of a gene product required for virulence or pathogenicity (i.e. the conditional-expression mutant cells in the test animals are sensitised). Control animals are challenged with the conditional-expression mutant strain and are fed a diet containing the candidate compound but lacking tetracycline. The virulence or pathogenicity of the conditional-expression A. fumigatus mutant strain in the test animals is compared to that in the control animals. For example, if a significant difference in growth is observed between the sensitised conditional-expression mutant cells (i.e. the cells in animals whose diet included tetracycline) and the non-sensitised cells (i.e. the conditional-expression mutant cells animals whose diet did not include tetracycline), the candidate compound may be used to inhibit the virulence or pathogenicity of the organism or may be further optimised to identify compounds which have an even greater ability to inhibit the virulence or pathogenicity of the organism. Virulence or pathogenicity may be measured using the techniques known in the art.

In another embodiment of the cell-based assays of the present invention, sensitised cells are obtained by reduction of the level activity of a polypeptide required for fungal viability using a mutation, such as a temperature-sensitive mutation, in the polypeptide. Growing such cells at an intermediate temperature between the permissive and restrictive temperatures produces cells with reduced activity of the gene product. It will be appreciated that the above method may be performed with any mutation which reduces but does not eliminate the activity or level of the gene product which is required for fungal viability. This approach may also be combined with the conditional-expression approach. In this combined approach, cells are created in which there is a temperature-sensitive mutation in the gene of interest and in which this gene is also conditionally-expressed.

When screening for inhibitors of an essential polypeptide, growth inhibition can be measured by directly comparing the amount of growth, measured by the optical density of the culture relative to uninoculated growth medium, in an experimental sample with that of a control sample. Alternative methods for assaying cell proliferation include measuring green fluorescent protein (GFP) reporter construct emissions, various enzymatic activity assays, and other methods well known in the art. Other parameters used to measure viability include e.g. colony forming units. The above method may be performed in solid phase, liquid phase, a combination of the two preceding media, or in vivo. Multiple compounds may be transferred to agar plates and simultaneously tested using automated and semi-automated equipment.

Cell-based assays of the present invention are capable of detecting compounds exhibiting low or moderate potency against the target molecule of interest because such compounds are substantially more potent on sensitised cells than on non-sensitised cells. The effect may be such that a test compound may be two to several times more potent, e.g. at least 10 times more potent, such as at least 20 times more potent, e.g. at least 50 times more potent, such as at least 100 times more potent, e.g. at least 1000 times more potent, or even more than 1000 times more potent when tested on the sensitised cells as compared to non-sensitised cells.

A mutant A. fumigatus strain that overexpresses an extracellular polypeptide can also be used to identify a compound that inhibits such a polypeptide. If the compound is cytotoxic, overexpression of the target polypeptide can make cells more resistant. Thus, the invention also relates to a method for identifying an inhibitor of an extracellular Aspergillus polypeptide selected from the group of SEQ ID NO:1-6 and 36 comprising the steps of:
a. providing two cells which differ in the activity of a polypeptide selected from the group of SEQ ID NO:1-6 and 36, wherein one cell contains a substantially wild-type copy number of said polypeptide and the other cell contains higher than wild-type activity of said polypeptide
b. determining the sensitivity of said cells to a putative inhibitor, for instance by a growth assay, and
c. determining whether or not said two cells are differently affected by the presence of said putative inhibitor.

Preferably, the two cells differ in the activity of a polypeptide selected from the group of SEQ ID NO:1, 2, 3, 5, 6, and 36, such as the polypeptide of SEQ ID NO:1, or the polypeptide of SEQ ID NO:2, or the polypeptide of SEQ ID NO:3, or the polypeptide of SEQ ID NO:5, or the polypeptide of SEQ ID NO:6 or the polypeptide of SEQ ID NO:36

As also overexpression of polypeptides that are not the cellular target of an inhibitor can make cells resistance to an inhibitor, inhibition of the target polypeptide of interest by said inhibitor will need to be verified by other means, such as e.g. a biochemical assay.

Overexpression may be achieved using strong promoters, e.g. the A. niger Pgla A promoter, the A. nidulans promoter alcA(p), or the constitutive promoters PGK-(phosphoglycero-kinase), GPD-(glucose-6-phosphate dehydrogenase) or ENO (enolase) promoters or regulated promoters such as ADH2, PHO5, GAL1, GAL10, CUP1 or HSP70. Other useful promoters include the ones described in Adams et al. (1998) Microbiol. Mol. Biol. Rev. 62, 35-54; Adams et al. (1988) Cell 54, 353-362; Andrianopoulos and Timberlake (1991) Plant Cell 3, 747-748; Gwynne et al. (1987) Gene 51:205-216; Lockington et al. (1985) Gene 33:137-149.

In addition to inhibitors of a biochemical or other cellular activity of an extracellular polypeptide, the cellular methods described above may identify compounds that reduce the expression level of a target, and thereby its copy number, e.g. by interfering with gene regulation.

In preferred embodiments of the any of the cell-based- or biochemical methods for identifying binding partners or inhibitors, the method is repeated for a plurality of candidate compounds.

In a further aspect, the invention relates to the mutant A. fumigatus strains used in the cell-based methods described herein, such as strains in which the gene encoding the extracellular polypeptide is placed under the control of a heterologous regulatable promoter, strains carrying temperature-sensitive alleles of the extracellular polypeptides, and strains overexpressing the extracellular polypeptides.

Other methods of interfering with fungal growth by targeting essential extracellular polypeptides include suppression of gene expression using specific antisense molecules, such antisense RNA or DNA, and using ribozyme molecules specific for mRNA encoding the essential extracellular polypeptides.

Diagnosis

In a further main aspect, the invention relates to a method of diagnosing Aspergillus infection comprising the steps of:
a. providing a sample from an individual,
b. contacting said sample with an indicator moiety specific for a polypeptide of the invention as defined herein, or specific for a polypeptide selected from the group of CssI (SEQ ID NO:1), hydrophobin (SEQ ID NO:2), GAPDH (SEQ ID NO:3), catalase A (SEQ ID NO:6) and isopropylmalate dehydrogenase B (SEQ ID NO:36), and
c. determining whether a signal has been generated by the indicator moiety.

In a preferred embodiment of this method, the polypeptide of the invention is a polypeptide selected from the group of SEQ ID NO:1, 2, 3, 5, 6, and 36 such as the polypeptide of SEQ ID NO:1, or the polypeptide of SEQ ID NO:2, or the polypeptide of SEQ ID NO:3, or the polypeptide of SEQ ID NO:5, or the polypeptide of SEQ ID NO:6, or the polypeptide of SEQ ID NO:36. In other preferred embodiments of this method, the indicator moiety is specific for a fragment selected from the group of fragments set forth in SEQ ID NO:7-34 and 37.

The indicator moiety is capable of binding to the target polypeptide of interest. In preferred embodiments, said indicator moiety is or comprises an antibody. Antibodies directed against a target extracellular polypeptide or fragment thereof can be used to detect the polypeptide in order to evaluate the abundance and pattern of expression of the polypeptide under various environmental conditions, in different morphological forms (mycelium, yeast, spores) and stages of an organism's life cycle.

Preferably, however, antibodies directed against a target polypeptide or fragment thereof can be used diagnostically to monitor levels of a target gene product in the tissue of an infected host as part of a clinical testing procedure, e.g., to, for example, diagnose a patient for Aspergillus infection or determine the efficacy of a given treatment regimen. In particular CssI is of considerable interest for diagnostic purposes. It appears that the protein is unique to A. fumigatus as no significant homologues to the protein have yet been detected through the use of immunological or sequence-based procedures. Furthermore, the cell-surface and secreted nature of the protein is also favourable feature from the point of view of detecting the protein in human fluids.

Detection using antibodies can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include Streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³H.

Various diagnostic assays employing the above indicator moieties can be set up to test samples for Aspergillus. Exemplary assays are described in detail in Antibodies: A Laboratory Manual, Harlow and Lane (eds.), Cold Spring Harbor Laboratory Press, 1988. Representative examples of such assays include: countercurrent immuno-electrophoresis (CIEP), radioimmunoassays, radioimmunoprecipitations, enzyme-linked immuno-sorbent assays (ELISA), dot blot assays, inhibition or competition assays, and sandwich assays, immunostick (dipstick) assays, simultaneous immunoassays, immunochromatographic assays, immunofiltration assays, latex bead agglutination assays, immunofluorescent assays, biosensor assays, and low-light detection assays (see U.S. Pat. Nos. 4,376,110 and 4,486,530; see also Antibodies: A Laboratory Manual, Harlow and Lane (eds.), Cold Spring Harbor Laboratory Press, 1988.

### EXAMPLES

### Example 1

### Identification of Peptides in Extracts of A. fumigatus

A number of protein purification procedures were used to facilitate identification of A. fumigatus proteins that are secreted, cell-surface exposed or cell-wall associated. Proteins were then identified from these extracts via mass spectrometry techniques.

Culture of A. fumigatus. A. fumigatus conidia (AfC) of strain NCPF 2140 or ATCC 46640 were routinely prepared by inoculation of malt agar plates with AfC and subsequent growth at 30° C. for 10 days.

Preparation of a diffusible extract from A. fumigatus conidia. A. fumigatus Diffusate (AfD) was routinely prepared as follows. AfC (2×10⁸) were added to water (0.5 ml) containing protease inhibitors (Roche, cat. no. 1 697 498) and the mixture was vortexed and then sonicated to solubilise the AfC. The resultant solution was incubated for 1 hour at 37° C., with shaking. AfD was then separated from washed spores by passage through a 0.2 μm filter, or, by centrifugation (3000×g) of washed spores and passage of the AfD through a 0.2 μm filter.

Preparation of surface-exposed protein extracts from A. fumigatus conidia. Washed AfC (2.0×10¹⁰) were resuspended in 1 ml of PBS (0.01M phosphate buffer, 0.0027 M potassium chloride, 0.137 M sodium chloride, pH 7.4) containing a reducing agent (10 mM Tris 2-carboxy-ethyl phosphine (TCEP)) and incubated for 20 min at room temperature. AfC were pelleted by centrifugation (20000×g, 30 min) and washed in PBS to remove TCEP before being resuspended in a trypsin-solution (seq. grade modified porcine trypsin, Promega cat. no. V5111, 20 μg/ml PBS) and incubated for 30 min at room temperature. AfC were then removed by centrifugation and filtration. To get rid of any conidia in the supernatant, the supernatant was purified using a YM-10 column (from Millipore, cat. no. 4206, 5000×g, 4° C. for 30 min.) and the supernatant was incubated over night at 37° C. with shaking at 40 rpm. The supernatant was concentrated using a SpeedVac concentrator, 1 μl was added to 6 μl 5% formic acid, and the resultant solution was analysed via mass spectrometry.

Preparation of cell wall extracts from A. fumigatus conidia. AfC solutions (20 ml; 1.8×10⁸ conidia/ml) were prepared in both PYG (C rich) (Peptone-yeast extract and glucose: 0.1% peptone, 0.1% g yeast extract and 0.3% glucose) and HBSS(C poor) (HANKS 1× from Gibco, Invitrogen (cat. no. 24020-083)) media. These solutions were vortexed for 3 min, sonicated for 5 min and then incubated for 4 hours at 37° C. with shaking (160 rpm). AfC were pelleted by centrifugation (6000×g, 30 min) and the supernatant from the HBSS incubation was collected and passed through a 0.2 μg/m filter. The supernatant from the PYG was discarded. Both AfC pellets were washed with 5 ml HBSS and pelleted as before. To each pellet 1 ml of lysis buffer (2% Triton, 1% SDS, 10 mM Tris (pH=2), 1 mM EDTA, 100 mM NaCl, 1 proteinase inhibitor tablet (Roche, cat. no. 1 697 498) and approx. 500 μl glass beads (200-300 microns) were added. The resultant solution was then incubated in a water bath sonicator for 40 min, vortexed for 30 min, chilled on ice for 5 min, and finally vortexed for another 30 min. Glass beads were then removed from the sample and conidial walls were sedimented by centrifugation at 1200×g for 10 min. The supernatant was removed and stored for future use.

Conidial wall enriched pellets were washed three times with 1 ml of cold distilled water, resuspended in 250 μl of 2% (w/v) SDS, 1% (w/v) 2-mercaptoethanol solution and boiled for 5 min. The resultant solution was centrifuged (10,000×g for 15 min), the supernatant was transferred to a new tube, and added to 1 ml ice-cold acetone prior to an overnight incubation at −30° C. Precipitated proteins were pelleted (20,810×g for 45 min) and dried in a SpeedVac for 15 min to remove residual acetone. Pellets were resuspended in ddH2O, and proteins were separated on an SDS-PAGE according to standard procedures. Resultant gels were then visualised via silver staining.

Analysis of A. fumigatus protein extracts by mass spectrometry. Analysis of A. fumigatus proteins separated by SDS-PAGE was performed as follows. Fragments of SDS-PAGE gels, corresponding to specific protein bands, were extracted and placed in sodium bicarbonate solution (50 mM NH₄HCO₃). These gel plugs were then washed twice with 50 mM NH₄HCO₃ in 50% ethanol for 30 min and dehydrated by incubation in 96% ethanol for 10 min. Reduction and alkylation was performed by incubating in reducing solution (50 mM DTT, 50 mM NH₄HCO₃) for 45 min at 56° C. followed by a 30 min room temperature incubation in alkylation solution (55 mM iodoacetamide, 50 mM NH₄HCO₃) in the dark. Two cycles of washing and dehydration were then performed prior to the addition of 10 μl trypsin solution (12.5 ng/μl trypsin in 50 mM NH₄HCO₃ (seq. grade modified porcine trypsin, Promega batch no. V51×14755007)). After 15 min an additional 20 μl of sodium bicarbonate solution was added and the digests were incubated overnight at 37° C. Samples were then extracted twice by 30 min incubations, with shaking, in 3 ml of 20% trifluoroacetic acid, and 20 μl of a solution containing acetonitrile (10%) and trifluoroacetic acid (1%). Both extracts were pooled, dried down, and resuspended in 9% of 5% formic acid prior to analysis via LC-MS.

Peptide and fragment mass tolerance was set to 200 ppm and 0.5 Da, respectively. Search parameters were adjusted to include oxidation of Met, the addition of alkyl of polyacrylamide groups to Cys, and trypsin was allowed to miss one cleavage site per peptide.

Search parameters for analysis of cell surface peptide fragments were adjusted to include oxidation of Met, trypsin was allowed to miss one cleavage site on each peptide; and, peptide and fragment mass tolerance was set to 100 ppm and 0.3 Da, respectively.

Following the identification of a peptide sequence, a TBLASTN was performed against A. fumigatus shotgun sequences in the public domain. This identified all shotgun sequences capable of encoding the peptide fragment. These shotgun sequences were then used to extract all other shotgun sequences that shared regions of homology of at least 40 bp in length with no less than 90% identity. All appropriate shotgun sequences were then formed into a contiguous sequence using Seqman. Resulting contigs were submitted to a GenScan search using maize, arabidopsis, and human parameters. Output predicted protein sequences were then compared with the encoding nucleotide sequence and with the sequences of protein homologues to facilitate the prediction of a potentially more accurate protein sequence. Resulting predicted nucleotide and peptide sequences were then entered into appropriate in-house databases and MASCOT searches were then rerun against a database containing these newly predicted proteins. For isopropylmalate dehydrogenase B, mismatches were found between the peptide found by peptide sequencing and the corresponding polypeptides predicted from the nucleotide sequences in the database. The mismatches may be due to differences between strains due to mutation or to sequencing errors. Furthermore, an MS instrument does not differentiate between a leucine and an isoleucine. Mutations in this region may have significant structural implications and alter the thermostability of the enzyme, as has been described for a homologous enzyme from Thermus thermophilus (Qu et al. 1997 Protein Eng. 10, 45-52).

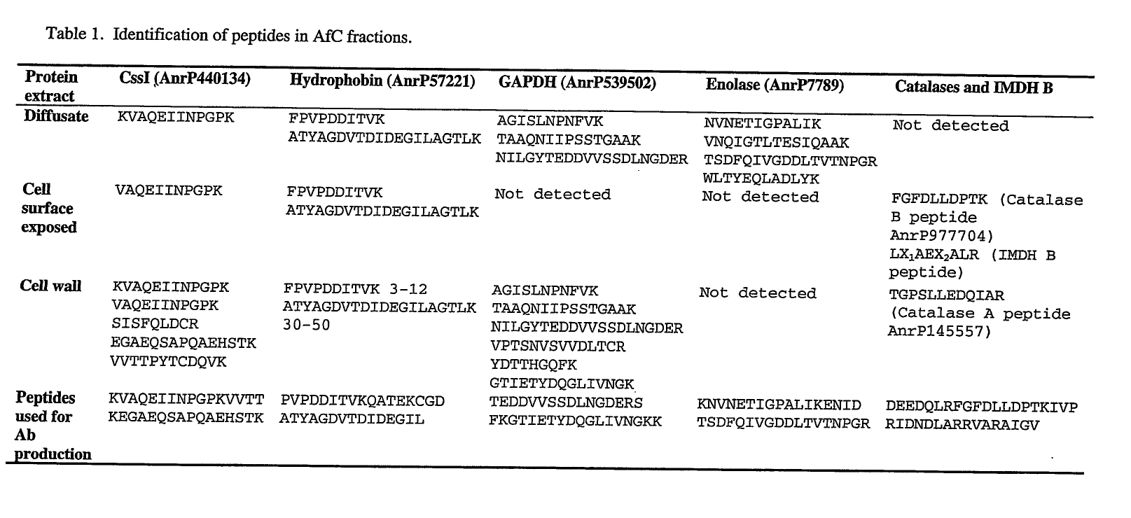

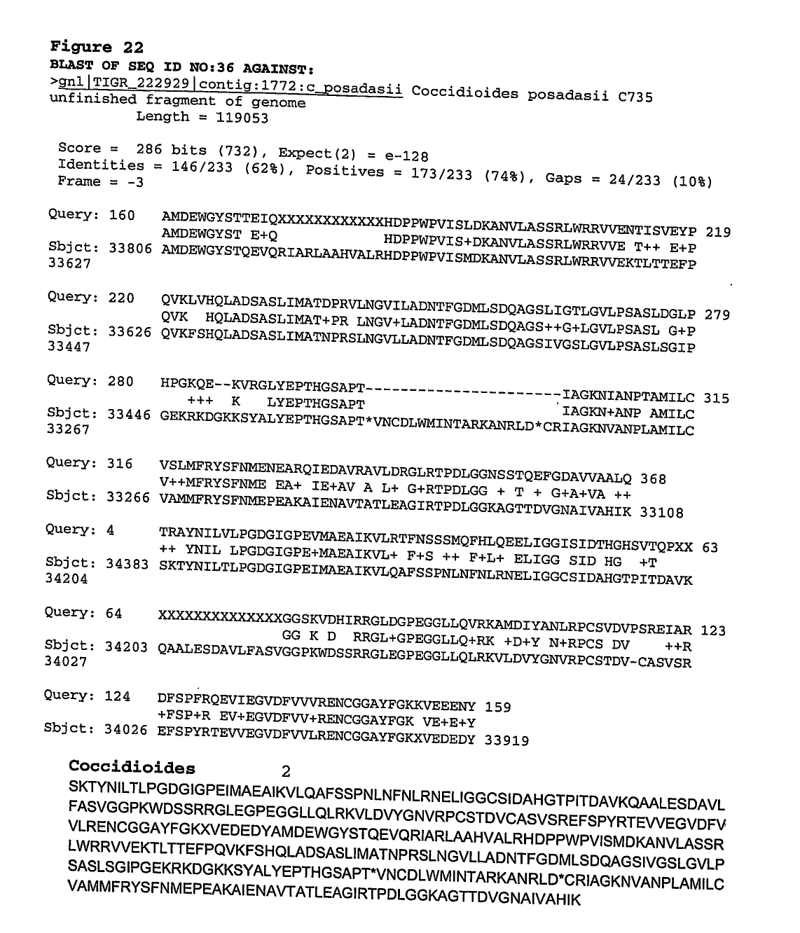

The peptides identified in the Diffusate, Cell-surface exposed, and Cell-wall fractions are shown in FIG. 26 (Table 1). The corresponding predicted protein sequences are given in FIG. 1. SEQ ID NO:38 and SEQ ID NO:39 are predicted polynucleotide sequences encoding isopropylmalate dehydrogenase B (SEQ ID NO:36).

Peptides from both hydrophobin and the hypothetical protein were identified in all three fractions indicating both to be cell-wall-associated proteins that are exposed on the surface of the AfC while also being secreted/released into the surrounding milieu. Based on these data we propose to name the newly identified, former hypothetical, protein Conidial Surface and Secreted protein I, CssI. It was also interesting to note the presence of GAPDH in the AfD and cell wall; enolase in the AfD; IMDH B in the cell-surface-exposed fraction and of cell wall located, and surface exposed variants of catalase. Since the procedures used to purify and identify these peptides are biased for proteins of high abundance, one can also conclude that they are expressed in relatively high copy numbers.

### Example 2

### Bioinformatic Analyses

SignalP predictions were performed using the parameters recommended for a eukaryotic protein, while Antigenicity index studies were performed using the default parameters determined by DNAStar. BLAST searches were performed using default parameters.

Analysis of CssI for the presence of a signal peptide.

The group who reported the original hypothetical sequence predicted an N-terminal signal peptide of 24 residues (NCBI entry CAD29600. Protein AfA35G10.07). However, a repeat of these studies using the SignalP program with default parameters (Nielsen et al. (1997) Protein Engineering 10, 1-6) indicates the presence of a 47-residue signal peptide with predicted signal cleavage occurring between A47 and R48.

Analysis of the predicted protein sequence of CssI.

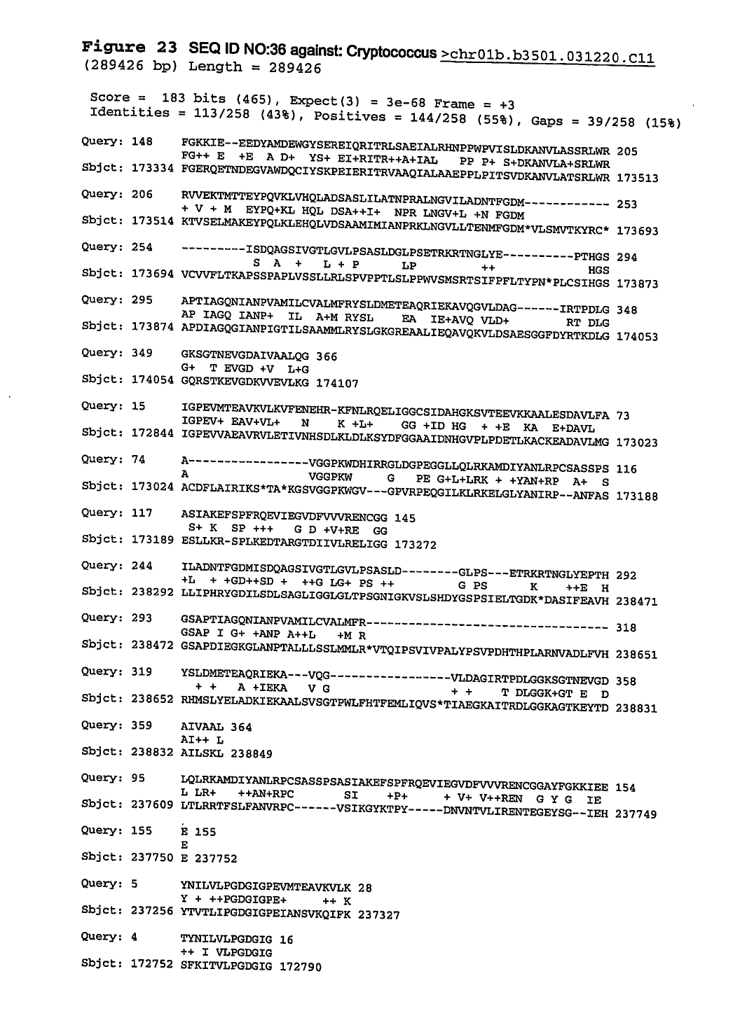

A brief overview of the sequence of this protein reveals the two most abundant residues to be E and Q, which comprise 9.62% and 8.64%, respectively, of all residues in the protein. A closer analysis revealed that 67% of charged residues (D, E, K, R) are located in the C-terminal half of the protein (see FIG. 27-Table 2), and 62% of hydrophobic residues (A, I, L, F, W, V) in the N-terminal half.

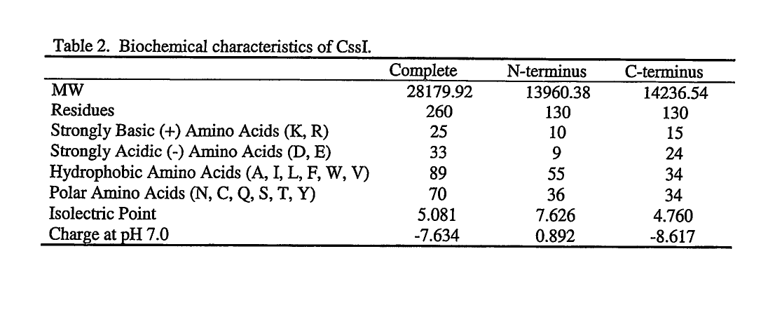

The sequence of CssI was analysed via the antigenicity index programme of Jameson and Wolf (1988). This programme predicted the C-terminal half of the protein to be most antigenic (see FIG. 2).

BLAST analysis of CssI revealed the absence of a protein with high homology. However, a number of proteins displayed low, yet significant, levels of homology. One such protein, ORF73 of Human herpesvirus 8, is the Latency associated nuclear antigen (LAN/LANA) that is used as a marker for Kaposi's sarcoma. It displays 26% identity and 46% similarity to the C-terminal half of CssI. This region of LANA is rich in Q and E repeats and is located in the middle of the protein. It has been suggested that similar regions of acidic repeats often function in transcriptional activation in viral and cellular transcription factors (Struhl, 1995, Annu. Rev. Genet. 29, 651-674). LANA has been shown to be capable of modulating both viral and cellular gene expression (Renne et al., 2001, J. Virol. 75, 458-468).

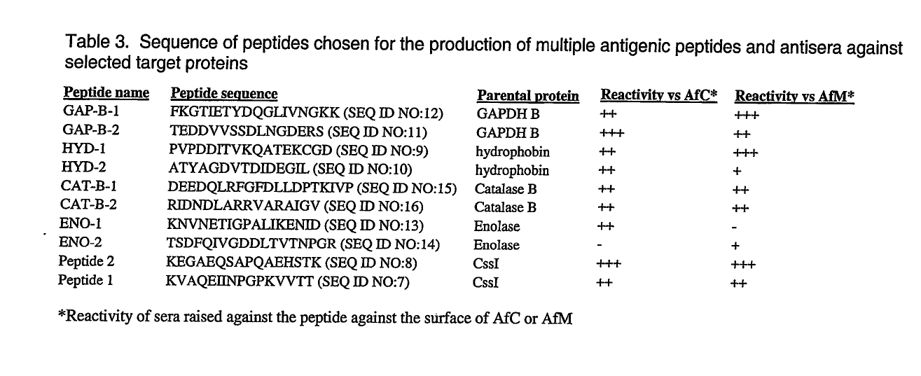

GAPDH sequences. An attempt to construct a gene sequence for this protein revealed the presence of at least three genes in Aspergillus fumigatus that are capable of expressing a GAPDH-related protein. These predicted proteins have been labelled GAPDH-A, GAPDH-B, and GAPDH-C. A number of differences exist between these two proteins (see FIG. 3). However, it is possible to conclude that only GAPDH-B has been identified to date. An inability to identify GAPDH-A or -C, to date, could be due to a number of reasons, e.g., a failure to be expressed under laboratory conditions; or, to the absence of an appropriate predicted protein sequence in the databases. The fact that only GAPDH-B was identified in cell wall and secreted preparations indicates that this version of the protein is likely to be primarily a cell-wall variant, and perhaps GAPDH-A and -C the cytoplasmic variants.

GAPDH-A and GAPDH-B share 73% identity and 85% similarity over a over a stretch of 269 residues. The more divergent GAPDH-C shares only 43% identity with both GAPDH-A and GAPDH-B. An analysis of all three sequences via an InterProScan revealed all three to be GAPDH sequences. However, only proteins A and B had sequences that matched to the active-site motif ([ASV]-S-C-[NT]-T-x(2)-[LIM]). This could imply that C does not function as a true GAPDH protein. Upon closer analysis of the sequences it is apparent that C contains a V residue, instead of [LIM], at the last position in the motif. Considering that V, L, M, and I are all hydrophobic residues, it is unlikely that the difference will result in a non-functional GAPDH active site.

Isopropylmalate dehydrogenase B sequence.

The closest homologues of the predicted isopropylmalate dehydrogenase sequence were previously described enzymes from A. niger, (accession number in NCBI database P87257 (77% identity over 363 aa)) and A. oryzae, (accession number in NCBI database BAC55906 (52% identity over 367 aa)).

Homology to human proteins and essentiality to A. fumigatus.

Of the protein mentioned above, neither CssI, IMDH B nor Hydrophobin have any significant human homologues. Both enolase (61% identical, 77% similar) and GAPDH (77% identical/83% similar), on the other hand, do have human homologues over the full length of the protein. However, due to the small size of any given epitope, and to the specificity of antibodies in general, it is likely that a suitable antibody can be found to distinguish A. fumigatus versions from the human versions.

Peptides for Antibody Production

The peptides found in the mass spectrometry analysis were used for antibody production. Some of them were extended with flanking residues from the predicted or known sequences.

### Example 3

### Generation and Properties of Anti-AfM and Anti-IMDH Antibodies

Methods

Phosphate Buffered Saline

PBS tablets (Sigma) were used to produce a final solution of 0.01M phosphate buffer, 0.0027 M KCl and 0.137 NaCl, pH 7.4 at 25° C.

Generation of Anti-Aspergillus fumigatus mycelia (anti-AfM) Antibodies

AfM-rich preparations were grown as follows: 10E5 AfC were added to 10 ml RPMI and incubated for approximately 10 hours at 37° C. AfM were then harvested by centrifugation and washed twice in PBS. These preparations were fixed by incubation in 3% formaldehyde for 30 min at room temperature, washed and 100 μg quantities injected into rabbits according to the following protocol.

Null sera was collected from New Zealand White female rabbits (4-6 months old, approximately 3 kg) prior to immunisation with 100 μg AfM and Freunds complete adjuvant. A booster was administered on day 14 in conjunction with Freunds incomplete adjuvant and again on day 28. The first and second bleeds were harvested on days 42 and 72, respectively, before the final bleed was taken on day 93. Subsequent analysis via immunofluorescent microscopy and western blotting demonstrated that the rabbit raised an Ab-based immune response against AfM.

IgG Purification

IgG was then purified from this sera using the MabTrap kit (Amersham) in accordance with the manufacturers instructions. Fab fragments were purified from IgG via Immunopure Fab kit (Pierce) again in accordance with the manufacturers instructions.

Adhesion Assay Protocol

A549 cells (1×10⁵) (DeHart et al. (1997) J. Infect Dis. 175(1):146-150) were seeded into the well of a Lab-Tek II 8 well chamber slide (Nalge Nunc International) and grown overnight. A solution of AfC (1×10⁸/ml) was prepared in RPMI medium, vortexed for 10 min and sonicated for 10 sec to suspend the AfC (in house studies have demonstrated that greater than 99% of AfC are viable after this step). The AfC population was then aliquoted, IgG preparations added where appropriate, and the samples incubated with shaking for 30 min at 37° C. A549 cells were prepared by washing three times with 400 μl F12K media. If required, purified protein was pre-incubated with A549 cells for 30 mins at 37° C. after which the cells were washed three times with 400 μl F 12K media. Finally, 190 μl of F 12K was added to each well, prior to the addition of 10 μl (1×10⁶ AfC) of the appropriate AfC solution. The samples were then incubated at 37° C. for 60 min. Unbound AfC were removed by washing 4 times with F12K media and A549 cells were detached using 400 μl trypsin EDTA. Following detachment the solutions were removed from the wells, stored in an eppendorf, and sonicated at medium intensity for 10 s to lyse mammalian cells. Finally, 0.03% Triton-X-100 was added to each solution to ensure that the AfC existed in a single cell form. The quantity of AfC in each sample was then determined by counting using a haemocytometer. All assays were run in triplicate.

Antigen Identification via IP with Anti-AfM Antibodies

Resultant IgG preparations were used, in conjunction with the Affigel kit (Biorad), to prepare an anti-AfM affinity column. This was done in accordance with manufacturers instructions. Thus, 20 mg of anti-AfM IgG was coupled to 200 μl affi-gel and the resultant column was rotated overnight with 100 mg of whole AfM lysate prepared in PBS. A column containing 200 μl affi-gel without IgG was also treated in the same manner. The supernatant was then removed and the gel washed twice with 0.5 ml PBS adjusted to 0.5 M NaCl. Washing was then performed with 0.5 ml PBS adjusted to 0.25 M NaCl and samples were eluted with 100 μl 0.2 M Glycin-HCl pH 2.5. Following storage on ice for 5 mins the samples were neutralised via the addition of 24 μl of 1 M Tris-HCl pH 8.5 and separated via SDS-PAGE. Amplification and cloning of the cDNA copy of IMDH B Total RNA was purified from 1×10⁹ AfC using the RNAEasy Kit (Qiagen) in accordance with manufacturers instructions. In order to design oligonucleotide primers to assist in the amplification of the cDNA, the sequence of the IMDH B was predicted using a number of bioinformatic steps. First, peptides identified via LCMS were BLASTED against the A. fumigatus shotgun database. All sequences matching the peptide were then aligned to produce a large contig sequence that was then input into the gene prediction program Genscan. This produced a number of predicted gene sequences and those that were predicted to encode the peptide identified via LCMS were selected for further study. IMDH B genes from other organisms that displayed homology to the A. fumigatus sequence were also used to assist in the selection of the most likely STOP and START codon. Thus, 18-mer oligos (F: 5′-ATGGTAACTACTTACAAC-3′ (SEQ ID NO:44); R: 5′-TGAACTACCCTGCAACGC-3′ (SEQ ID NO:45)) were designed and used to amplify a cDNA copy of the Af IMDH B gene using the Superscript One Step kit from Invitrogen in accordance with manufacturers instructions. Following amplification the product was cloned into pBAD using the TOPO TA cloning kit (Invitrogen) and sequencing was used to confirm the sequence and orientation of the insert.

Sequencing

Sequence reactions were performed using the BigDye terminatorv3.1 (Applied Biosystems) kit in accordance with the manufacturers instructions.

Expression of Heterologous Proteins

Upon identification of a clone containing the desired sequence a number of experiments were performed to identify the optimal expression conditions for the heterologous protein. These revealed optimal expression when induced with 0.02% arabinose for 4 hours. Proteins were then purified via utilisation of pBAD encoded his-tag sequence.

Preparation of Bacterial Lysates

Each liter of E. coli culture was induced for four hours with 0.02% arabinose and bacterial cells were harvested by centrifugation (5000 rpm, 15 min). The bacterial pellet was then resuspended in 25 ml cold native buffer (20 mM NaPO₄, 500 mM NaCl, 25 mM imidazole, pH 7.4), and the solution supplemented with 2 protease inhibitor tablets (ROCHE, complete-EDTA free Protease inhibitors) and 625 μl lysozyme (25 μg/11) before incubation on ice for 1 h. The solution was then sonicated for 4 min, subjected to a cycle of freeze thaw, and 7.5 μl benzonase (362 units/μl) added prior to incubation on ice for 20 minutes. In order to assist in the removal of insoluble components the solution was centrifuged at 5000×g for 20 min and the supernatant harvested. This step was repeated twice more and the cleared lysate then analysed via SDS-PAGE.

Preparation of Nickel Sepharose Columns for Purification of his-Tagged Proteins

Purification of recombinant proteins was performed using Probond resin (invitrogen), a nickle-sepharose based resin that utilises the pBAD-encoded his-tag on the heterologously-expressed recombinant protein. All centrifugation steps were performed at 800×g for 2 min. The resin was resuspended by inverting and gently tapping the container and 2 ml slurry of resin was added to an Econo-Pac Chromatography Columns (BioRad, Cat. No. 732-1010) for equilibration. The column was centrifuged and the supernatant discarded. The resin was then washed with 10 ml sterile water, and again the supernatant discarded following centrifugation. Next, the resin was resuspended in 10 ml Native buffer (20 mM NaPO₄, 500 mM NaCl, 25 mM imidazole, pH 7.4), centrifuged and the supernatant discarded. This process was repeated twice. Finally the resin was resuspended in 1 ml native buffer giving a final volume of 2 ml.

Application of Lysate, Washing and Elution of Purified Protein

The bacterial lysate containing the recombinant protein was added to the column containing the equilibrated resin and the mixture incubated on a roller for 100 min at 4° C. The mixture was then centrifuged at 800×g for 2 min and the supernatant poured off and saved. The resin was then allowed to settle, the column plug was removed and the liquid allowed to flow through. The run through from this step, and all subsequent steps, was collected for SDS-PAGE analysis. The column was then washed with 20 ml Native buffer (20 mM NaPO₄, 500 mM NaCl, 25 mM imidazole, pH 7.4) a total of 5 times and the protein eluted protein by applying 20 ml Native elution buffer (20 mM NaPO₄, 500 mM NaCl, 250 mM imidazole, pH 7.4) and collecting ten 2 ml aliquots. SDS-PAGE analysis was then performed to determine the outcome of the procedure.

Gel Filtration Purification of Nickel Sepharose Purified IMDH B

Probond purified IMDH B was first desalted via dialysis in PBS (dialysis tubing 12-14000 Daltons, Visking). Gel filtration was performed using a HiPrep 16/60 Sephacryl S-200 High Resolution column (Amersham Biosciences). Preparation, equilibration and cleaning of the column was completed according to the manufacturers instructions. Protein purification was performed with a Tris running buffer (10 mM Tris+0.15 M NaCl, pH 8) at an approximate flow rate of 0.25 ml/min. Batches of his-tagged protein (10-20 mg) were added to the column and were eluted using 80 ml running buffer, a void volume of approximately 40 ml was calculated and 18×2 ml fractions were collected.

Generation of Anti-IMDH B Antisera

Probond-purified protein was used to immunise New Zealand White female rabbits (4-6 months old, ˜3 kg). Pre-immune sera were harvested on day 1 prior to immunisation with 100 μg of protein in the presence of Freunds complete adjuvant. Further immunisations were also carried out on days 28 and 49 in the presence of Freunds incomplete adjuvant. Blood samples were taken on days 28, 42, 69 and finally on 87. IgG and Fab fragments were prepared as previously described.

Immunofluorescent Microscopy

Immunofluorescence microscopy slides were first cleaned in a detergent solution and rinsed in distilled water. The slides were then incubated in coating solution (0.1% gelatin [w/v], 0.01% [w/v] chrome alum) and allowed dry at room temperature. Harvested AfC spores were placed onto the well of a treated immunofluorescence microscopy slide and the slide was washed three times with PBS to remove unbound AfC. If AfM were required 30 μl RPMI was added to the surface of the well and the slide incubated at 37° C. for 12-14 hours before washing with PBS. IgG (30 μl, 1:500 dilution in PBS) was then added to each well and incubated at 37° C. for 30 min in a moist environment. The wells were washed three times with PBS before the addition of Alexa Fluor 488 goat-anti-rabbit IgG (30 μl of a 1:400 dilution prepared in PBS) to each well. Again the slides were incubated at 37° C. for 30 min in a moist environment, washed three times with PBS and then fixed in 3% formaldehyde for 30 min at room temperature. The slides were washed three times in PBS, allowed to dry and then a drop of Sigma oil was added to the surface and a coverslip applied. This was sealed with nail polish and the slides stored at 4° C. in the dark until use. All wells were scanned in both bright and fluorescent fields

Determination of the Ability of IgG to Inhibit Conidial Germination

AfC were resuspended in RPMI medium and the concentration adjusted to give a solution containing 1×10⁶ AfC ml. 0.5 ml of this solution was then added to an eppendorf and 10 μl of each IgG/serum sample added to 3 aliquots of AfC. Normal fresh rabbit serum was added to one aliquot, heat-inactivated (60° C. for 30 min) serum to the second, and 10 μl PBS to the third. The samples were then incubated for 7 hours at 200 rpm. The total number of cells was then counted and the number of conidia that have germinated, or started to produce a germ tube, determined. Percent filamentation equals the number of cells with germ tubes divided by the total number of cells.

Generation of sera Against Predicted Antigenic Peptides of Target Proteins

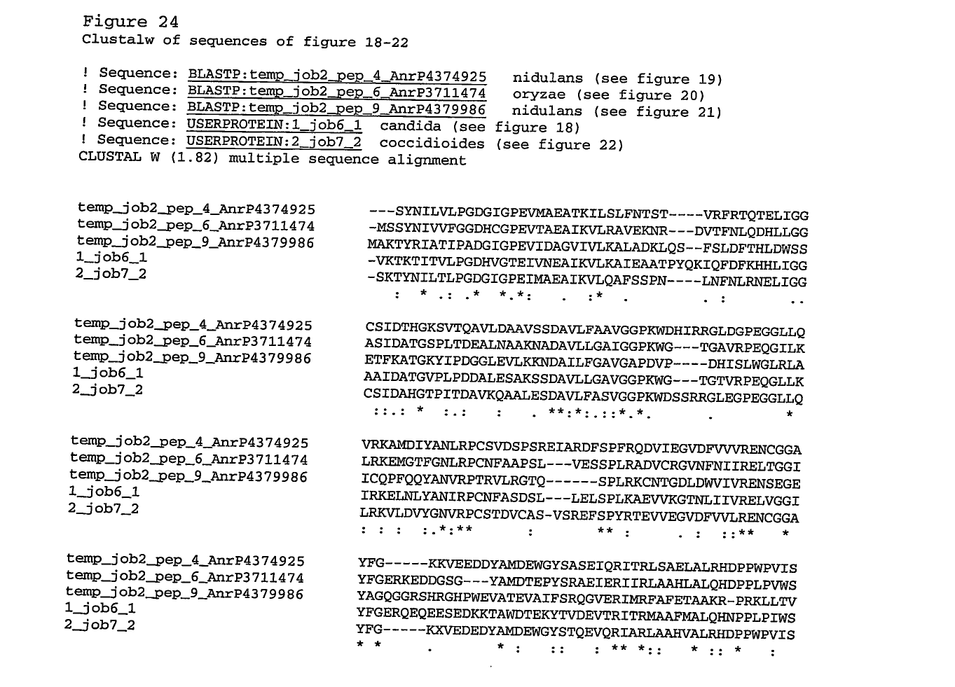

Analysis of target proteins was performed with the aim of identifying a number of antigenic peptides within each target sequence. The peptides chosen (see FIG. 28—table 3) correspond to those that were identified during mass spectrometry procedures used to identify surface exposed peptides (see FIG. 26—table 1) and to those predicted to be most antigenic (Jameson, B. A. & Wolf, H (1988) Comput Appl Biosci 4, 181-186).

Peptides and antisera were produced in accordance with Sigma's standard Custom Peptide Synthesis and rabbit immunisation protocols. The peptides were produced as part of their multiple antigenic peptides (MAP) service. The immunisation protocol used is as follows: on day 0 pre-immune sera was collected from New Zealand white rabbits which were then immunised with 200 μg of MAP in complete freunds adjuvant (CFA). A second immunisation was performed on day 14 with 100 μg in Incomplete freunds adjuvant (IFA). This immunisation was repeated on days 28, 42, 56 and 70. Bleeds were taken on days 35, 49, 63, and finally on day 77.

MS Analysis

In-gel Digestion

The gel bands are washed two times with 25 mM NH₄HCO₃/50% ethanol. The cysteine residues are reduced (DTT) and alkylated (iodoacetamide), followed by 2 cycles of wash and dehydration of the gel bands. Washing buffer is 50 mM NH₄HCO₃ and dehydrating agent is ethanol. After the last dehydration step, the digestion is started by addition of protease, dissolved in suitable digestion buffer. Default enzyme is trypsin for which a 50 mM NH₄HCO₃/10% Acetonitrile digestion buffer is used. The digestion takes place over night at 37° C. The resulting peptide pool are extracted with TFA and formic acid and analysed by mass spectrometry. The handling of all samples takes place in a dust free environment with a minimum of manual handling to avoid keratin contamination.

MALDI-TOF Sample Preparation and Analysis

The peptide mixture extracted after in-gel digestion is analysed by a fully automated procedure on an Ultraflex Bruker MALDI-TOF mass spectrometer. A peptide mass fingerprint is recorded and each spectrum is annotated and internally calibrated using trypsin auto-digest peaks. Sample preparation:
1. A 96 tip robot is used to prepare a Bruker 384 polished steel target with a nitrocellulose/□-cyano-4-hydroxy-cinnamid acid (HCCA) matrix.
2. Approximately 5% of the sample is added to the matrix (1.5 μl).
3. When the sample is completely dry, it is washed with 0.1% TFA and is now ready to be analysed in the Bruker mass spectrometer.
   Database Search

The resulting peak list is used in a database search that is performed using Mascot software (MATRIX SCIENCE). The database is a non-redundant database (NRDB) based on data from NCBI.

Results

Antigen Identification via IP with Anti-AfM Antibodies

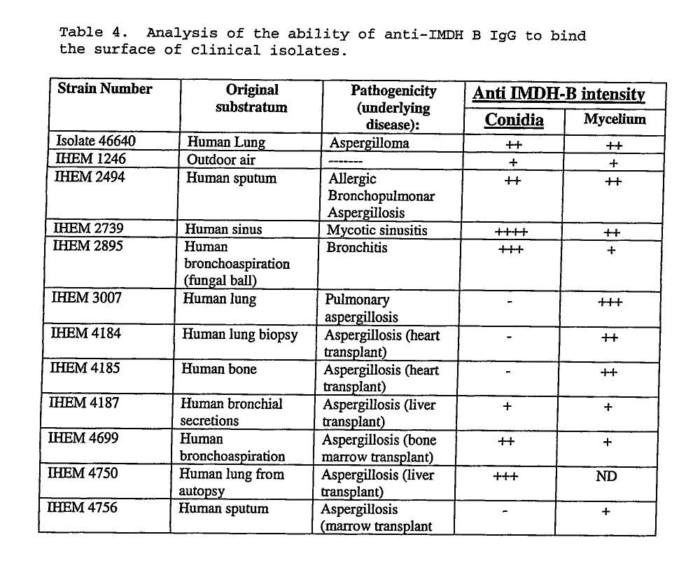

An affinity column consisting of anti-AfM IgG was used to immunoprecipitate the most antigenic proteins from an AfM lysate. Eluted samples were separated via SDS-PAGE and then analysed via LC MSMS analysis, which revealed 3-isopropylmalate dehydrogenase B (IMDH B) to be the prime target of anti-AfM antibodies, suggesting IMDH B to be the main antigen of AfM (FIG. 4).

Anti-AfM antibodies bind to the surface of AfM giving a considerably higher signal than the null sera.

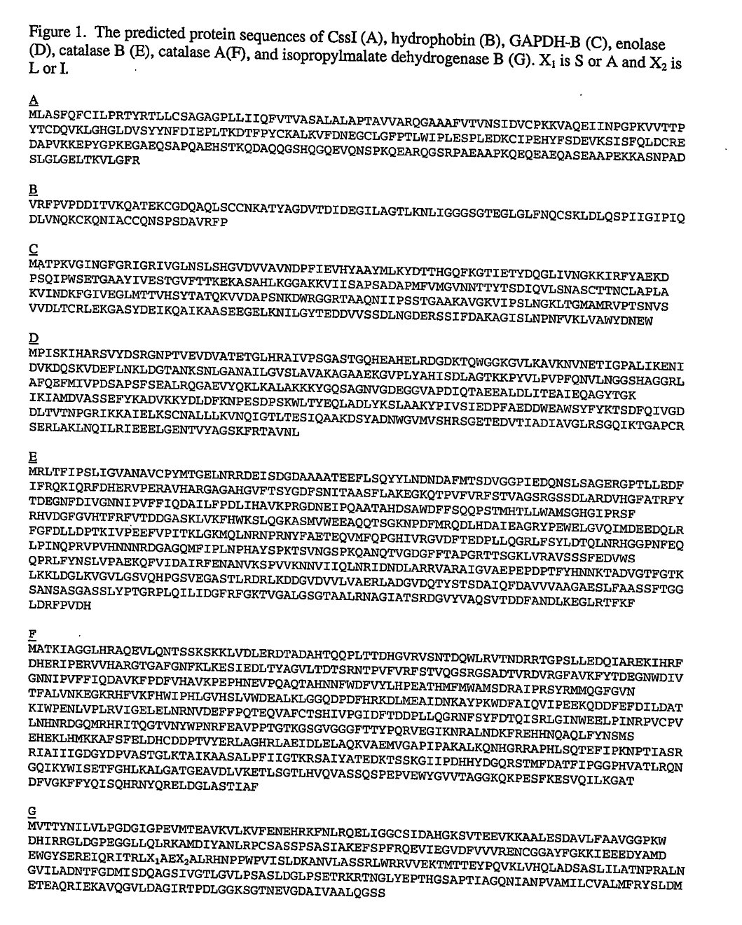

IFM studies revealed that anti-AfM antibodies bound to both AfM and AfC giving a higher signal than was observed with null sera. This again suggests IMDH B to be a major surface antigen of both conidia and mycelia (FIG. 5).

Anti-AfM Fab Fragments Reduce the Proportion of AfC that Adhere to A549 Cells

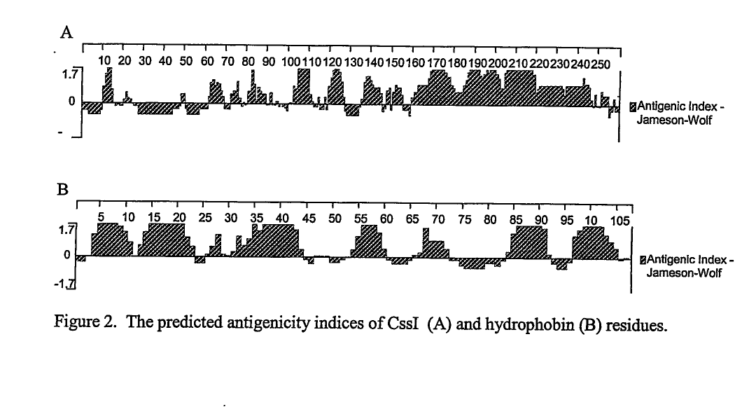

Having substantiated that IMDH B is a major surface antigen of both AfC and AfM it was decided to analyse the potential ability of anti-IMDH B antibodies (in the form of anti-AfM antisera) to interfere with the pathogenicity of AfC. Thus, adhesion assays were performed where the ability of AfC, pre-incubated in IgG, to bind to lung epithelia was measured. These studies indicated that antibodies, in the form of Fab fragments, were indeed capable of reducing adhesion of the organism to lung epithelia (FIG. 6). Without being bound by any theory, it is believed that the Fabs possess the ability to reduce adhesion to lung epithelia by interfering with the interaction between a receptor and its ligand, either by binding an adhesin and preventing its function, or by binding to the AfC surface and sterically inhibiting the function of an underlying adhesin. While IgG molecules may also possess such properties, the Fc region is capable of binding Fc receptors on the surface of the mammalian cells, an event which has the potential to elevate adhesion of AfC.

Cloning, Expression and Purification of Recombinant IMDH B

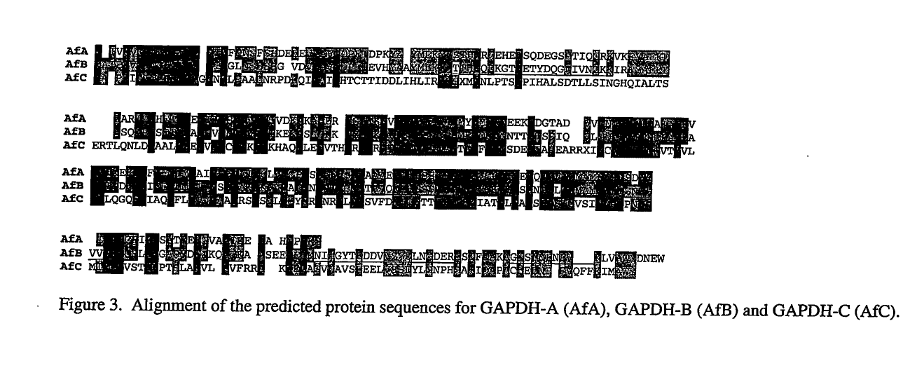

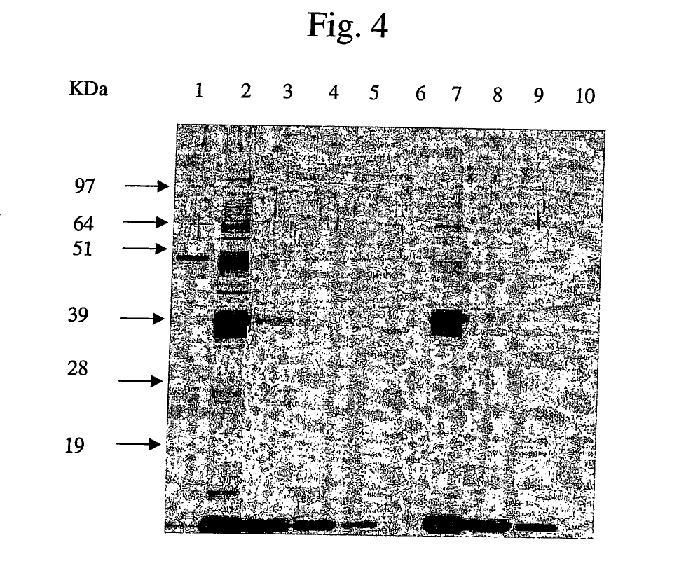

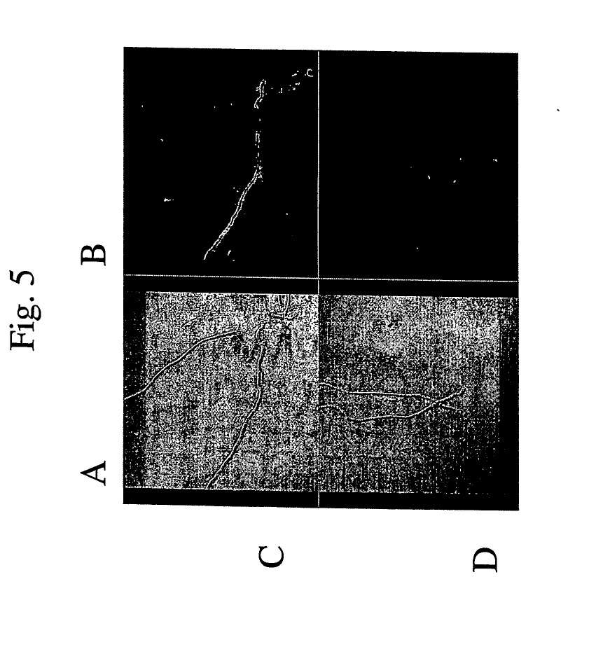

A copy of cDNA encoding IMDH B was successfully cloned into pBAD and the sequence of the insert confirmed (see SEQ ID NO:46 and SEQ ID NO:47). The protein was expressed following induction with 0.02% arabinose for 4 h and purified to a high degree of purity using nickel speharose and gel filtration columns (FIG. 7). The nickel sepharose purified fraction was used to immunise rabbits and this procedure was successful in generating anti-IMDH B antibodies (FIG. 8). IFM analysis also confirmed that anti-IMDH B IgG bound to the surface of AfC and AfM (FIG. 9), thus confirming the surface localisation of IMDH B.

IFM Analysis of Clinical Isolates

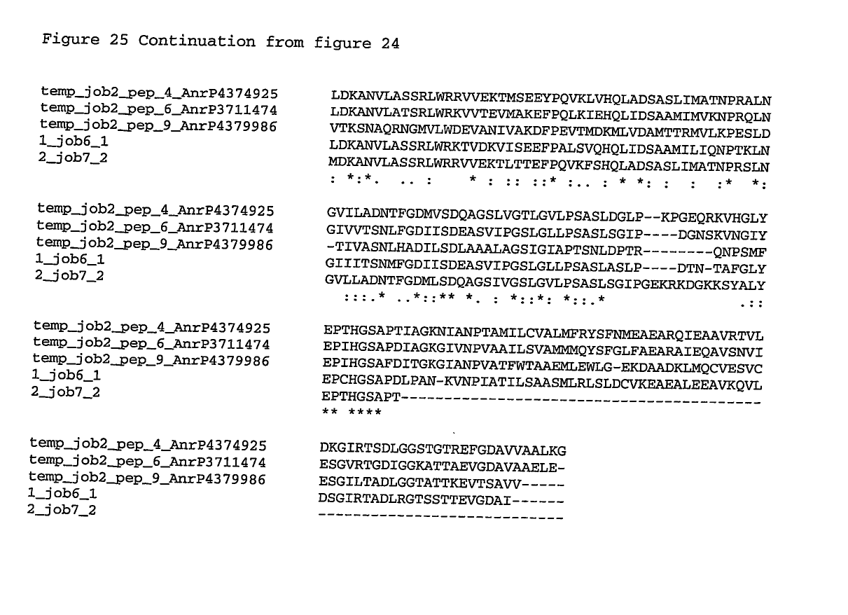

IFM analysis was performed on a number of clinical isolates to confirm the presence or absence of surface expressed IMDH B. All clinical isolates were purchased from the Belgian Co-ordinated Collections of Micro-organisms (BCCM™). Their features and reactivity to anti-IMDH B IgG are listed in FIG. 29 (Table 4).

Post-translational Modifications

A number of studies were performed with the aim of identifying the presence of post-translational modifications in the recombinant IMDH B protein. These studies took a number of forms. First, MALDI analysis was performed with the aim of identifying all of the predicted tryptic fragments of the protein. Thus the protein was digested with trypsin and analysed via MS. Using this method it was possible to get 84% sequence coverage: (SEQ ID NO:47 (vector-derived sequences in italics, sequences identified by MALDI analysis underlined)).

MGSGSGDDDDKLALMVTTYNILVLPGDGIGPEVMTEAVKVLKVFENEHRK
FNLRQELIGGCSIDAHGKSVTEEVKKAALESDAVLFAAVGGPKWDHIRRG
LDGPEGGLLQLRKAMDIYANLRPCSASSPSASIAKEFSPFRQEVIEGVDF
VVVRENCGGAYFGKKIEEEDYAMDEWGYSEREIQRITRLSAEIALRHNPP
WPVISLDKANVLASSRLWRRVVEKTMTTEYPQVKLVHQLADSASLILATN
PRALNGVILADNTFGDMISDQAGSIVGTLGVLPSASLDGLPSETRKRTNG
LYEPTHGSAPTIAGQNIANPVAMILCVALMFRYSLDMETEAQRIEKAVQG
VLDAGIRTPDLGGKSGTNEVGDAIVAALQGSSKGELEGKPIPNPLLGLDS
TRTGHHHHHH.

SDS-PAGE and time of flight analyses of the predicted 44233 Da Protein revealed apparent masses of 45000 Da and 44235 Da, respectively. In total, these studies did not provide any evidence indicating the presence of post-translational modifications.

Adhesion Assays Results

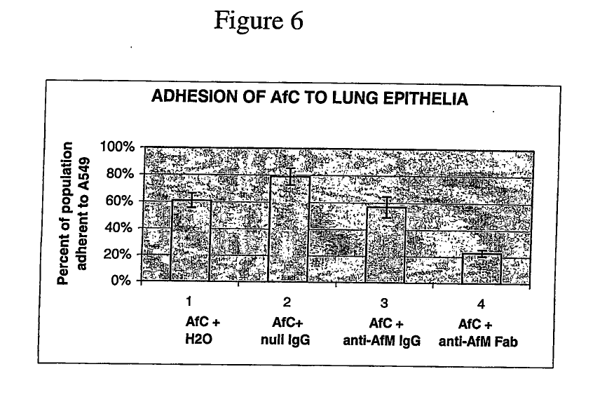

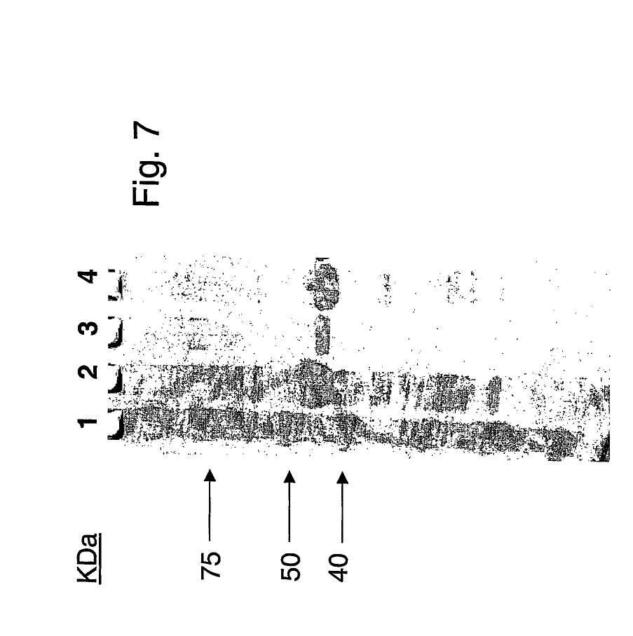

A number of studies support the theory that IMDH B acts as an adhesin. Thus, pre-incubation of AfC with anti-IMDH B Fab fragments produced a decline in the number of adherent AfC in comparison to AfC that had been pre-incubated in the presence of Fab fragments isolated from pre-immune sera (see FIG. 10). Furthermore, pre-incubation of A549 epithelia with varying quantities of recombinant IMDH B, followed by washing to remove unbound protein, suggested that the protein had sequestered the receptor on the surface of the A549 cells and thus prevented the AfC from adhering (see FIG. 11).

Germination Inhibition Experiments

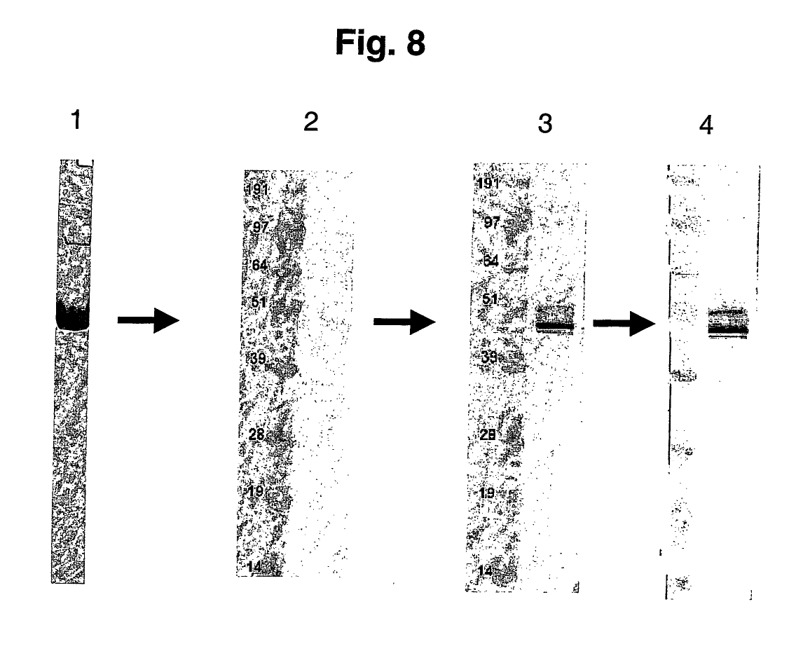

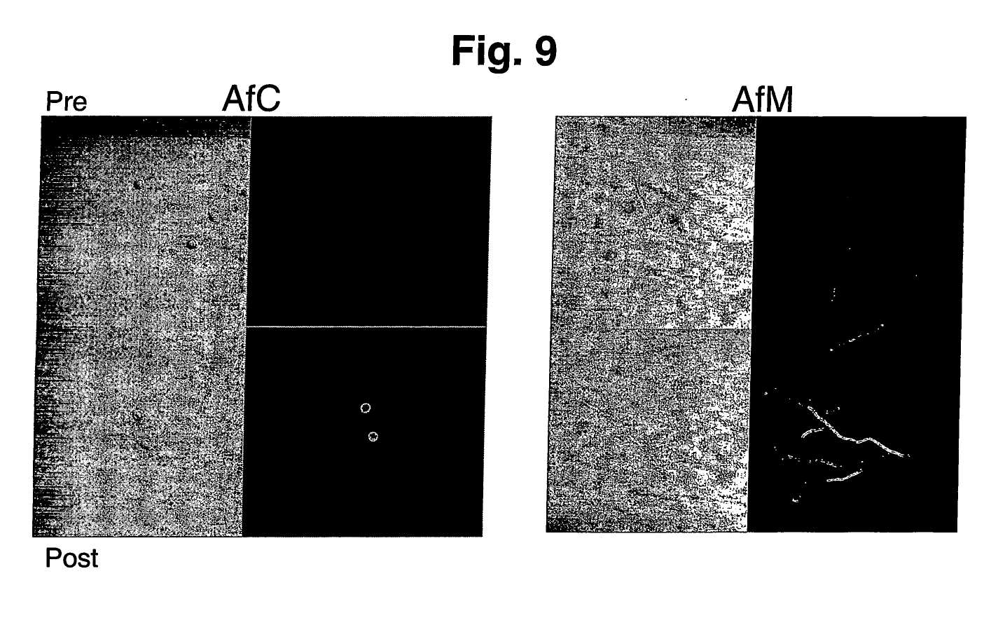

A number of experiments were performed to determine the ability of anti-IMDH B antibodies to reduce germination of AfC in the presence of normal or heat-inactivated rabbit serum. These experiments revealed that only anti-IMDH B heat-inactivated sera in the presence of fresh rabbit sera possessed the ability to reduce the rate of germination of AfC (see FIG. 12). Further studies revealed that purified anti-IMDH B IgG in the presence of normal rabbit serum also possessed the ability to reduce germination of AfC in comparison to null IgG (see FIG. 13).

Identification of a Second Copy of IMDH B

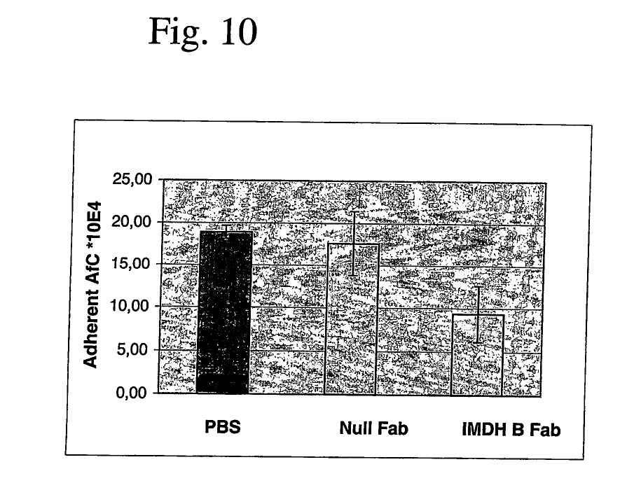

Further bioinformatic analysis revealed the presence of a second region on the Af genome that displays homology to the IMDH B gene and that is predicted to encode a protein that is 50% ID and 63% similar to IMDH B 1 (for alignment, see FIG. 14). The sequence of the respective gene and protein were predicted by BLAST analysis. Features of this protein are displayed below.

Predicted Features of Gene/Protein Compared to ACE5033 (IMDH B 1)

| | IMDH B 2 | ACE5033 |
|---|---|---|
| Number of exons | 5 | 3 |
| Size of ORF | 1093 bp | 1107 bp |
| Protein length | 363 residues | 368 residues |
| Protein size | 38723 Da | 39773 Da |
| pI | 5.26 | 5.32 |
| Identity to ACE5033 | 53% (over full length) | — |
| Similarity to ACE5033 | 69% (over full length) | — |

IMDH B 2 contains the final predicted ORF sequence (SEQ ID NO: 40):

atgccgtcatataacattgtcgttttcgctggggaccactgtggtccgga
ggtaagttcggtcctgcgcgtcatcgagaagtgccgtgacgatgctacct
tcaacctccaggatcaattgctcggtggtgtaagttcgatcgatgctacc
ggatctccccttaccgacgaagctcttaacgccgcaaagaacgccgatgc
cgttctcctcggtgccattggcggtcccaaatggggcactggcgccgtcc
gccccgaacagggcctcctccgtctgcgcaaggagatgggcacattcggt
aacctccgcccctgcaacttcgccgccccgtcgctggtcgacggctcccc
tctccgccccgaagtctgccgcggcgtcgacttcaacattatccgcgaac
tgaccggtggcatctacttcggcgaccgcaaggaggacgacggcagcggc
ttcgccatggacacggagccgtactcccgcgcggagatcgagcgcatcac
ccgccttgcggcccacctcgctctgcagcacaacccccctcttcccgtgt
ggagcttggacaaggccaacgtcctcgcgacgagccggctgtggcggaag
accgtgacggaggtcatggccaaggagttcccccagctcaaggtggagca
ccagctcattgactccgcggccatgatcatggtcaaggagcctagaaagc
ttaacggtattgttgtcactagcaacctgttcggtgacatcatcagtgat
gaagccagcgttatccctggttctctgggactcttgcccagcgcaagctt
gagcggcattcctgacggaaagaccaaggtcaatggtatctatgagccta
ttcacggttctgcccctgacattgccggcaagggcatcgttaaccccgtc
gccgccattctctctgtcgccatgatgatgcagtactccctgaaccgtat
ggatgacgccagggccatcgagacggccgtccgcaatgtgatcgaggccg
gtatccgcactgccgatattggcggcaagtcgacaactagcgaggtcggt
gacgctgttgctgccgagctggagaagctgttgaagcaatagt

This encodes the following protein (SEQ ID NO: 41):

MPSYNIVVFAGDHCGPEVSSVLRVIEKCRDDATFNLQDQLLGGVSSIDAT
GSPLTDEALNAAKNADAVLLGAIGGPKWGTGAVRPEQGLLRLRKEMGTFG
NLRPCNFAAPSLVDGSPLRPEVCRGVDFNIIRELTGGIYFGDRKEDDGSG
FAMDTEPYSRAEIERITRLAAHLALQHNPPLPVWSLDKANVLATSRLWRK
TVTEVMAKEFPQLKVEHQLIDSAAMIMVKEPRKLNGIVVTSNLFGDIISD
EASVIPGSLGLLPSASLSGIPDGKTKVNGIYEPIHGSAPDIAGKGIVNPV
AAILSVAMMMQYSLNRMDDARAIETAVRNVIEAGIRTADIGGKSTTSEVG
DAVAAELEKLLKQ

### Example 4

### Studies Performed on Additional ACE Targets

Generation of sera Against Predicted Antigenic Peptides of Target Proteins

Following the identification of a number of novel proteins on the surface of the AfC it was decided to further these studies through the production of antisera that had been raised against multiple antigenic peptides (MAP) of the novel molecules. Thus, two peptides from each chosen target were selected and the information relayed to Sigma who produced the peptides and antisera in accordance with their Custom Peptide Synthesis and rabbit immunisation protocols. The peptides chosen (see FIG. 28—table 3) correspond to those that were identified during mass spectrometry procedures (see FIG. 26—table 1) and to those predicted to be most antigenic using the guidelines supplied by Sigma.

IFM Analysis with sera Raised Against MAP Molecules

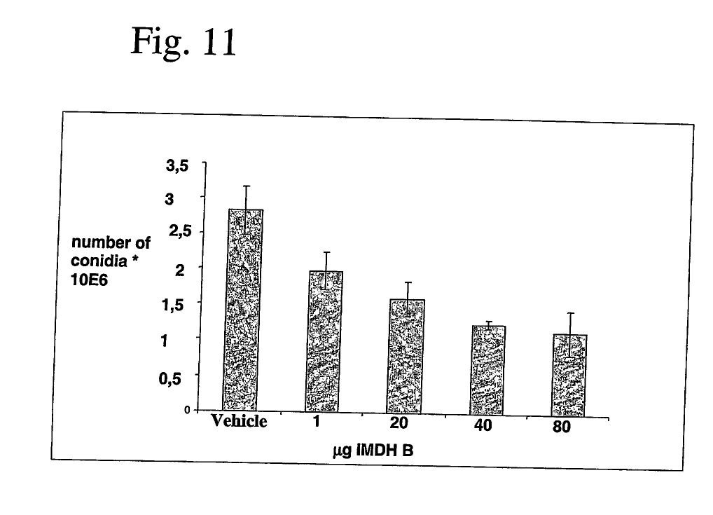

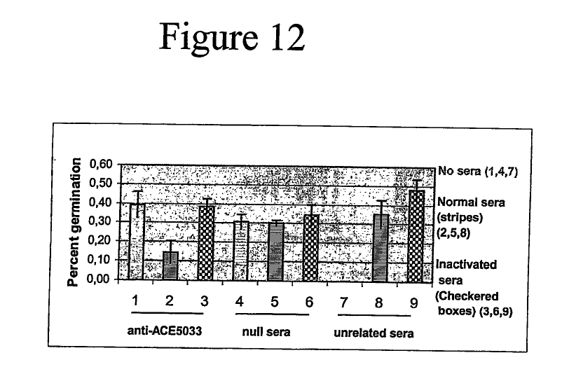

Following the production of MAPs and the corresponding antisera, IgG was purified and used in IFM experiments to assist in the confirmation of the surface expression of the various target molecules. The results of these IFM experiments are summarised in table 3 (FIG. 28) and exemplified for GAP-B-2 in FIG. 15. Sera raised against the GAP-B-2 molecule of GAPDH2 were also tested against a number of the clinical isolates. Reactivity of this sera against the surface of the clinical isolates (exemplified in FIG. 16, other not shown) supports the surface localisation of GAPDH2 within isolates other than ATCC 46640.

Cloning, expression and purification of recombinant enolase; plus detection of native enolase.

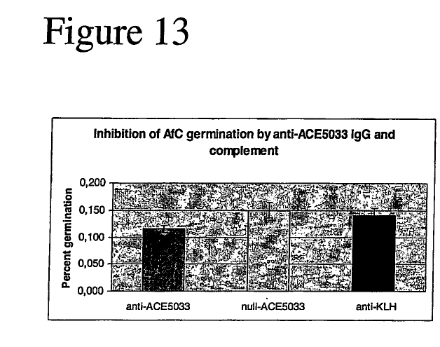

The enolase gene of A. fumigatus was cloned and the protein expressed using the same protocols detailed for IMDH B, the only difference being the sequence of the forward (5′-ATGCCTATCTCCAAGATC-3′ (SEQ ID NO:42)) and reverse primers (5′-CAGGTTGACGGCAGT-3′ (SEQ ID NO:43)). The sequence of the cloned cDNA molecule was confirmed using standard procedures) (SEQ ID NO:48 and SEQ ID NO:49). Expression of the recombinant protein was confirmed using anti-his antibodies (FIG. 17(A)). Furthermore, the protein was shown to react with anti-MAP sera raised against ENO-2 (FIG. 17(A)). Having confirmed that the anti-MAP sera binds recombinant enolase it was decided to test the sera against isolated membrane and wall fractions from AfC. These studies revealed the protein to exist in the cell membrane of AfC (FIG. 17(B)).

### Example 5

### Alignment of Aspergillus fumigatus IMDH B with Homologous Polypeptides from Other Fungi

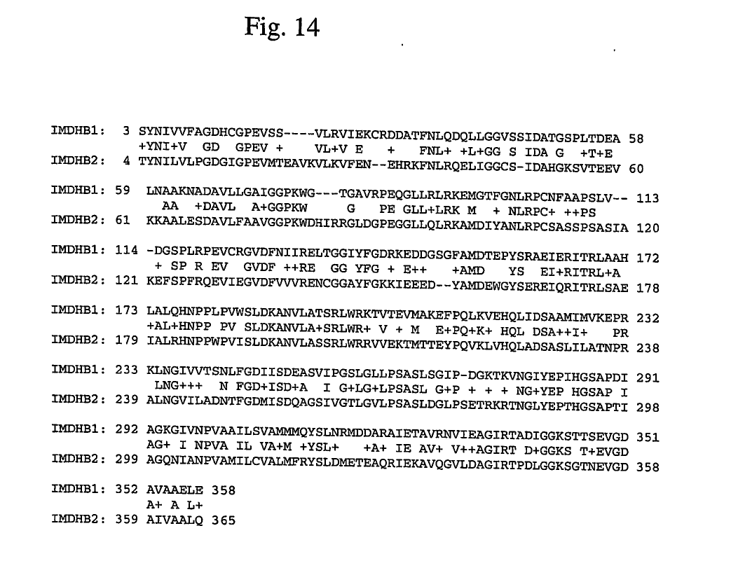

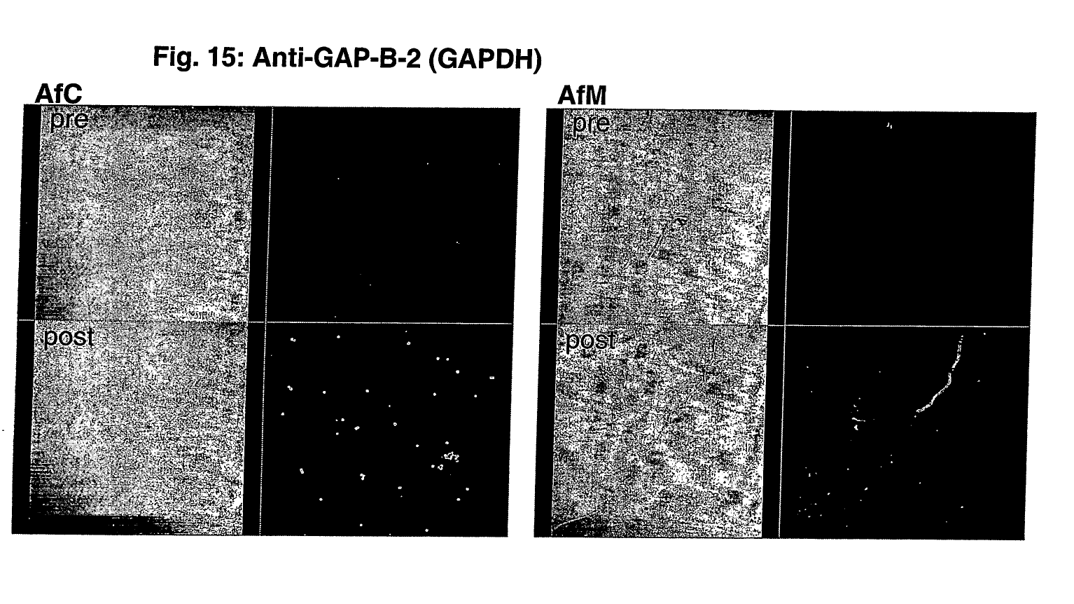

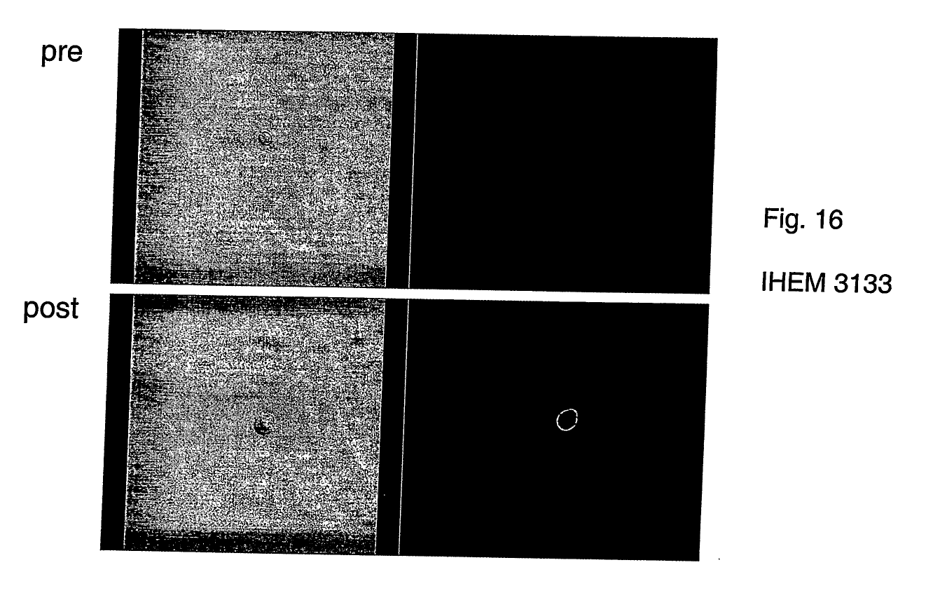

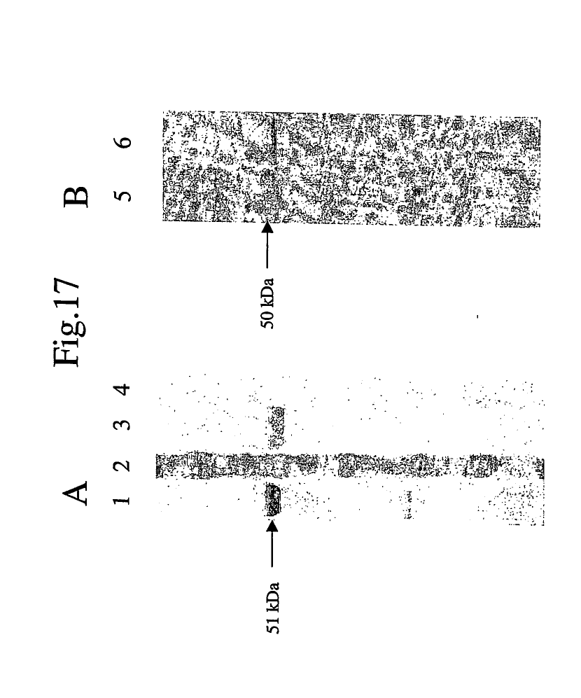

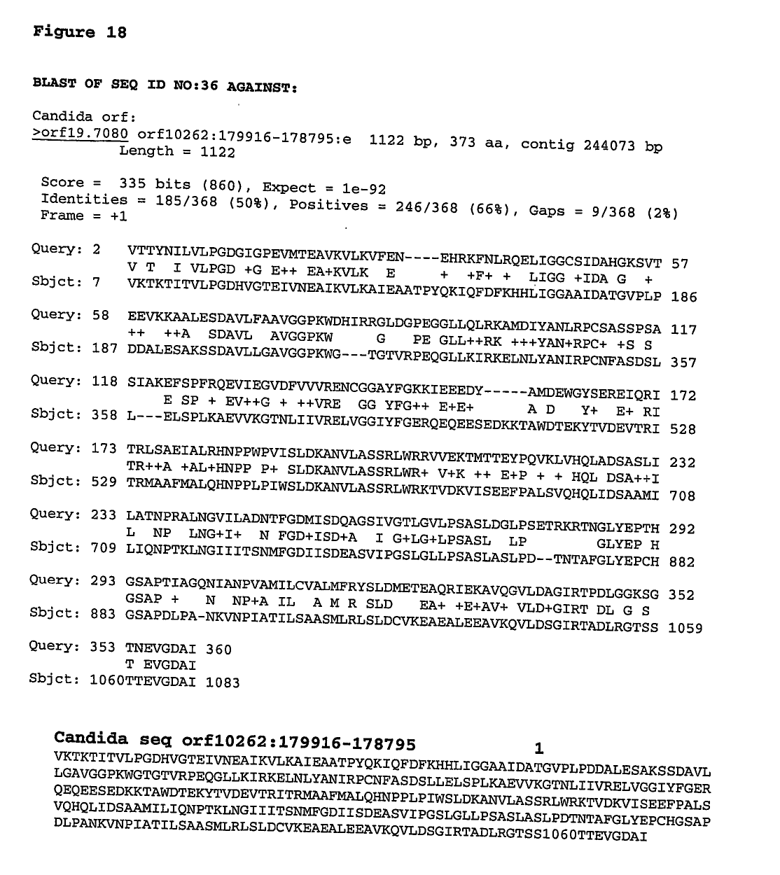

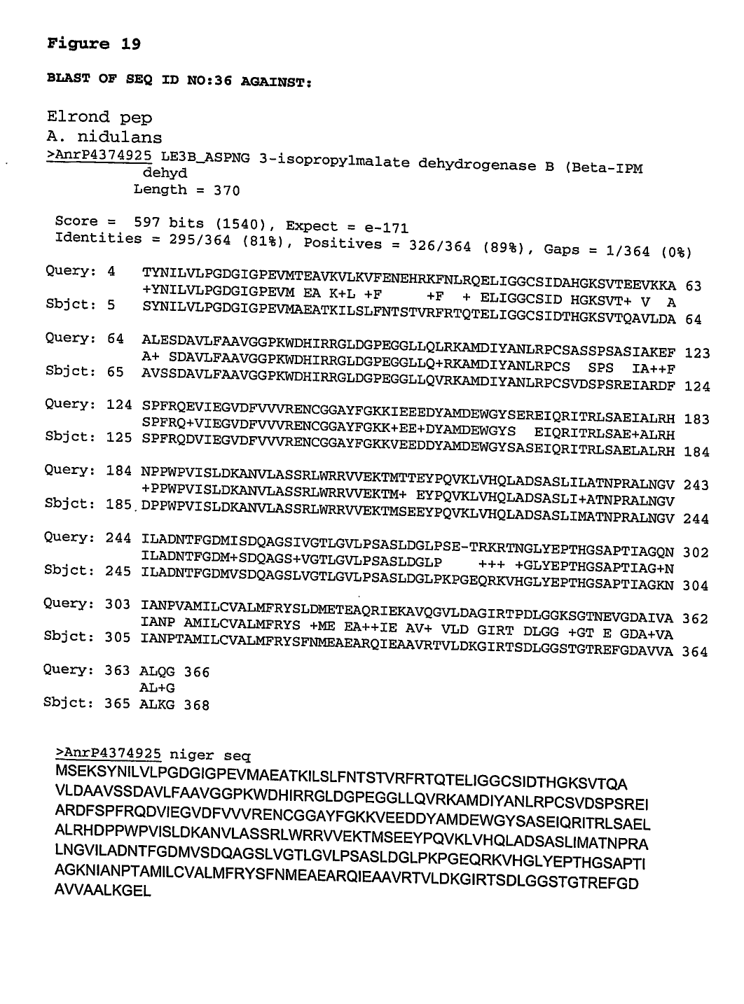

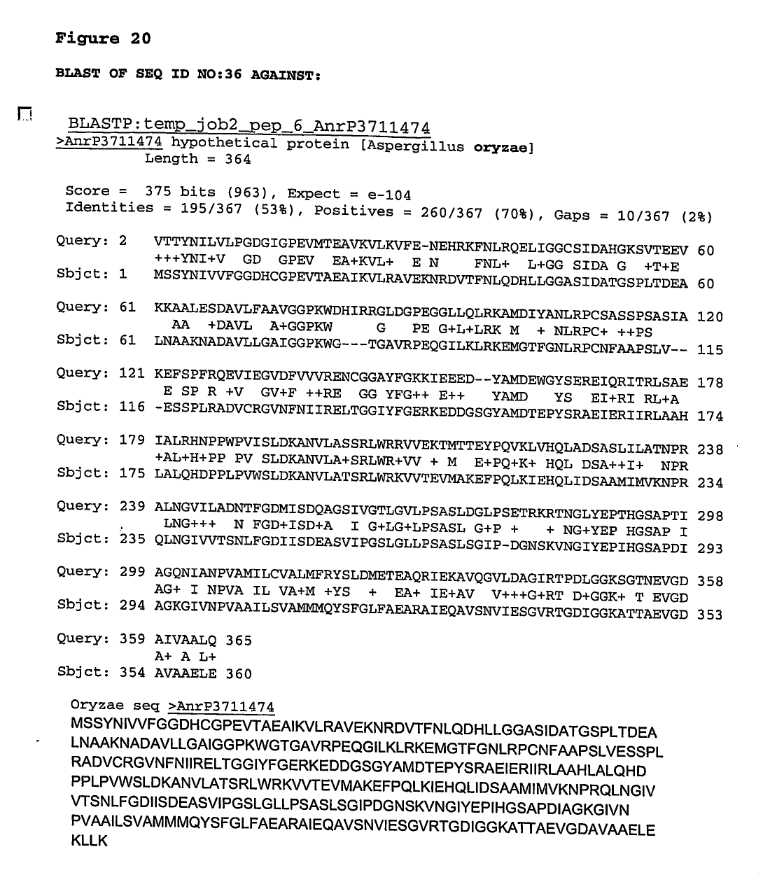

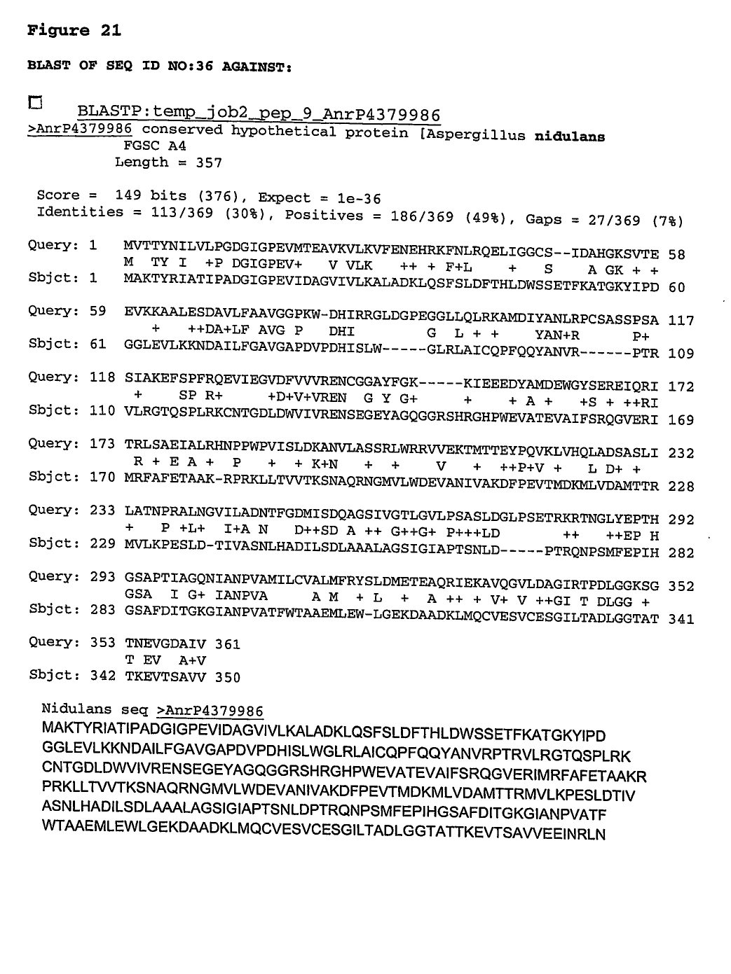

The sequence of Aspergillus fumigatus IMDH B (SEQ ID NO:36) was compared with homologous sequences from other fungi in order to find areas of high sequence identity. FIGS. 18-25 show alignments of SEQ ID NO:36 with homologous polypeptides.

While certain embodiments of the present invention have been described, it will be understood that various changes may be made in the above inventions without departing from the scope of the invention. It is intended that all matter contained in the above description or shown in the accompanying drawings shall be interpreted as illustrative and not in a limiting sense.

1. An isolated antibody capable of binding an extracellular Aspergillus fumigatus polypeptide selected from the group consisting of: isopropylmalate dehydrogenase B (SEQ ID NO: 36), CssI (SEQ ID NO: 1), hydrophobin (SEQ ID NO: 2), GAPDH-B (SEQ ID NO: 3), and catalase A (SEQ ID NO: 6).

2. (canceled)

3. The antibody of claim 1, wherein the antibody is selected from the group consisting of: IgG, IgA, IgE, IgM and IgD, wherein IgG preferably is IgG1.

4. The antibody of claim 1, wherein the antibody is capable of binding an intact Aspergillus fumigatus cell.

5-6. (canceled)

7. The antibody of claim 1, wherein the antibody is polyclonal.

8. The antibody of claim 1, wherein the antibody is monoclonal.

9. The antibody of claim 8, wherein the antibody is a chimeric, human or humanized antibody.

10. The antibody of claim 8, wherein the antibody is a human antibody.

11. The antibody of claim 1, wherein the antibody is purified.

12. The antibody of claim 1, wherein the antibody is further capable of binding a homologous polypeptide, wherein the homologous polypeptide has a sequence identity of at least 39%, such as 42% or more, 48% or more, 68% or more, 80% or more, or 90% or more, to a polypeptide selected from the group consisting of: isopropylmalate dehydrogenase B (SEQ ID NO: 36), CssI (SEQ ID NO: 1), hydrophobin (SEQ ID NO: 2), GAPDH-B (SEQ ID NO: 3), and catalase A (SEQ ID NO: 6).

13. The antibody of claim 12, wherein said homologous polypeptide originates from:
an Aspergillus species, such as Aspergillus fumigatus, Aspergillus nidulans, Aspergillus niger, or Aspergillus oryzea,
Neurospora crassa,
Saccharomyces cerevisiae,
a Candida species such as Candida albicans,
a Coccidioides species, such as Coccidioides posadasii, or Coccidioides immitis,
a Cryptococcus species, such as Cryptococcus neoformans var. neoformans,
a Fusarium species,
a Pneumocystis species,
a Penicillium species, or
Histoplasma capsulatum.

14. The antibody of claim 13, wherein said homologous polypeptide originates from:
an Aspergillus species, such as Aspergillus fumigatus, Aspergillus nidulans, Aspergillus niger or Aspergillus oryzea,
Candida albicans,
Coccidioides posadasii, or
Cryptococcus neoformans var. neoformans.

15. The antibody of claim 14, wherein said homologous polypeptide originates from an Aspergillus species, such as Aspergillus fumigatus, Aspergillus nidulans, Aspergillus niger or Aspergillus oryzea.

16. The antibody of claim 15, wherein said homologous polypeptide originates from Aspergillus fumigatus.

17. The antibody of claim 16, wherein the said homologous polypeptide is the polypeptide of SEQ ID NO: 41.

18-20. (canceled)

21. The antibody of claim 1, wherein the antibody is capable of binding a polypeptide selected from the group consisting of: isopropylmalate dehydrogenase B (SEQ ID NO: 36), CssI (SEQ ID NO: 1) and catalase A (SEQ ID NO: 6).

22. The antibody of claim 1, wherein the antibody is capable of binding a polypeptide selected from the group consisting of: isopropylmalate dehydrogenase B (SEQ ID NO: 36) and CssI (SEQ ID NO: 1).

23. The antibody of claim 1, wherein the antibody is capable of binding isopropylmalate dehydrogenase B (SEQ ID NO: 36).

24. The antibody of claim 23, wherein the antibody is capable of binding an epitope which comprises one or more of the residues of a region of SEQ ID NO: 36 selected from the group consisting of: Ser67-Leu71, Ala74-Trp80, Ser191-Arg205, Leu268-Leu273, His292-Pro296, Glu355-Ile360, Asp193-Glu209, Asp193-Ala199, Ile15-Val19, Val75-Trp80, Pro11-Glu18 and the region defined by SEQ ID NO: 37, preferably an epitope which is entirely consisting of residues comprised within said region.

25. A pharmaceutical composition comprising an antibody as defined in claim 1 and a pharmaceutically-acceptable carrier.

26. (canceled)

27. A method for the treatment or prevention of fungal infection, comprising administering to an individual a pharmaceutically-effective amount of an antibody as defined in claim 1.

28. The method of claim 27, wherein the fungal infection is an Aspergillus infection, preferably an Aspergillus fumigatus infection.

29. The method of claim 27, wherein the fungal infection being treated or prevented is selected from the group consisting of: invasive aspergillosis, aspergilloma, and allergic aspergillosis, such as allergic bronchopulmonary aspergillosis.

30. A composition comprising one or more Aspergillus fumigatus polypeptides selected from the group consisting of:
polypeptides comprising SEQ ID NO: 36, fragments thereof and variants thereof, fragments of SEQ ID NO: 1 of less than 259 amino-acid residues in length, such as less than 200, preferably less than 150, such as less than 100, such as less than 50, such as less than 25 amino-acid residues in length comprising one or more residues of the amino-acid sequences set forth in SEQ ID NOS: 7, 8, 17, 26, 28, 29 and/or 30 and variants of said fragments;
fragments of SEQ ID NO: 2 of less than 106 amino-acid residues in length, such as less than 75, preferably less than 50, such as less than 25 residues in length comprising one or more residues of the amino-acid sequences set forth in SEQ ID NOS: 9, 10, 18 and/or 19 and variants of said fragments;
polypeptides comprising SEQ ID NO: 3, fragments thereof and variants thereof, with the proviso that if the polypeptide is a fragment of SEQ ID NO: 3, that this fragment is not the fragment set forth in SEQ ID NO: 35;
fragments of SEQ ID NO: 4 of less than 437 amino-acid residues in length, such as less than 200, preferably less than 100, such as less than 75, such as less than 50, such as less than 25 amino-acid residues in length comprising one or more residues of the amino-acid sequences set forth in SEQ ID NOS: 13, 14, 23, 24 and/or 25 and variants of said fragments;
fragments of SEQ ID NO: 5 of less than 727 amino-acid residues in length, such as less than 400, such as less than 200, preferably less than 100, such as less than 75, such as less than 50, such as less than 25 amino-acid residues in length comprising one or more residues of the amino-acid sequences set forth in SEQ ID NOS: 15, 16 and/or 27 and variants of said fragments; and
fragments of SEQ ID NO: 6 of less than 748 amino-acid residues in length, such as less than 400, such as less than 200, preferably less than 100, such as less than 75, such as less than 50, such as less than 25 amino-acid residues in length comprising one or more residues of the amino-acid sequences set forth in SEQ ID NO: 34 and variants of said fragments.

31. An Aspergillus fumigatus polypeptide selected from the group consisting of:
polypeptides comprising SEQ ID NO: 36, fragments thereof and variants thereof, fragments of SEQ ID NO: 1 of less than 259 amino-acid residues in length, such as less than 200, preferably less than 150, such as less than 100, such as less than 50, such as less than 25 amino-acid residues in length comprising one or more residues of the amino-acid sequences set forth in SEQ ID NOS: 7, 8, 17, 26, 28, 29 and/or 30 and variants of said fragments;
fragments of SEQ ID NO: 2 of less than 106 amino-acid residues in length, such as less than 75, preferably less than 50, such as less than 25 residues in length comprising one or more residues of the amino-acid sequences set forth in SEQ ID NOS: 9, 10, 18 and/or 19 and variants of said fragments;
polypeptides comprising SEQ ID NO: 3, fragments thereof and variants thereof, with the proviso that if the polypeptide is a fragment of SEQ ID NO: 3, that this fragment is not the fragment set forth in SEQ ID NO: 35;
fragments of SEQ ID NO: 4 of less than 437 amino-acid residues in length, such as less than 200, preferably less than 100, such as less than 75, such as less than 50, such as less than 25 amino-acid residues in length comprising one or more residues of the amino-acid sequences set forth in SEQ ID NOS: 13, 14, 23, 24 and/or 25 and variants of said fragments;
fragments of SEQ ID NO: 5 of less than 727 amino-acid residues in length, e.g. less than 400, such as less than 200, preferably less than 100, such as less than 75, such as less than 50, such as less than 25 amino-acid residues in length comprising one or more residues of the amino-acid sequences set forth in SEQ ID NOS: 15, 16 and/or 27 and variants of said fragments; and
fragments of SEQ ID NO: 6 of less than 748 amino-acid residues in length, such as less than 400, such as less than 200, preferably less than 100, such as less than 75, e.g. less than 50, such as less than 25 amino-acid residues in length comprising one or more residues of the amino-acid sequences set forth in SEQ ID NO: 34 and variants of said fragments.

32. The polypeptide of claim 31, wherein the polypeptide is a fragment comprising one or more residues of the amino-acid sequences set forth in SEQ ID NOs: 7-27 and/or 37, or a variant of said fragment.

33. The polypeptide of claim 32, wherein the polypeptide is a fragment comprising one or more residues of the amino-acid sequences set forth in SEQ ID NOs: 7-16, or a variant of said fragment.

34. The polypeptide of claim 32, wherein the polypeptide is a fragment comprising one or more residues of the amino-acid sequences set forth in SEQ ID NOs: 17-25 and/or SEQ ID NO: 14, or a variant of said fragment.

35. The polypeptide of claim 32, wherein the polypeptide is a fragment comprising one or more residues of the amino-acid sequences set forth in SEQ ID NO: 18, 19, 26, 27, and/or 37, or a variant of said fragment.

36. A polynucleotide encoding a polypeptide as defined in claim 31.

37. An expression vector comprising a polynucleotide as defined in claim 36.

38. A host cell transformed or transfected with a polynucleotide as defined in claim 36.

39. A pharmaceutical composition comprising a polypeptide as defined in claim 31 and a pharmaceutically-acceptable carrier.

40. (canceled)

41. A method for the immunization of a mammal against fungal infections, comprising the administration of a polypeptide as defined in claim 31.

42. The method of claim 41, wherein said mammal is a human being.

43. A method for raising specific antibodies to a polypeptide selected from the group consisting of polypeptides set forth in SEQ ID NO: 1, 2, 3, 6 and 36 in a non-human mammal comprising the steps of:
a. providing a polypeptide selected from the group consisting of: isopropylmalate dehydrogenase B (SEQ ID NO:36), CssI (SEQ ID NO:1), hydrophobin (SEQ ID NO:2), GAPDH (SEQ ID NO:3), and catalase A (SEQ ID NO:6), or a polypeptide as defined in claim 31, or a cell expressing any of these polypeptides,
b. introducing a composition comprising said polypeptide or said cell into said animal,
c. raising antibodies in said animal, and
d. isolating and optionally purifying the antibodies.

44. The method of claim 43, wherein the raising of antibodies is done in a transgenic animal which is capable of producing human antibodies.

45. The method of claim 43, wherein the polypeptide that is provided is isopropylmalate dehydrogenase B (SEQ ID NO: 36) or a fragment thereof, or a variant of said polypeptide.

46. The method of claim 43, wherein the polypeptide that is provided is CssI (SEQ ID NO: 1) or a fragment thereof, or a variant of said polypeptide.

47. The method of claim 43, wherein the polypeptide that is provided is hydrophobin (SEQ ID NO: 2) or a fragment thereof, or a variant of said polypeptide.

48. The method of claim 43, wherein the polypeptide that is provided is GAPDH-B (SEQ ID NO: 3) or a fragment thereof, or a variant of said polypeptide.

49. The method of claim 43, wherein the polypeptide that is provided is catalase A (SEQ ID NO: 6) or a fragment thereof, or a variant of said polypeptide.

50. A method for identifying a binding partner of a polypeptide selected from the group consisting of: isopropylmalate dehydrogenase B (SEQ ID NO:36), CssI (SEQ ID NO:1), hydrophobin (SEQ ID NO:2), GAPDH-B (SEQ ID NO: 3), enolase (SEQ ID NO: 4), catalase B (SEQ ID NO: 5) and catalase A (SEQ ID NO: 6), comprising the steps of:
a. providing a polypeptide as defined in claim 31 or a polypeptide selected from the group consisting of: isopropylmalate dehydrogenase B (SEQ ID NO: 36), CssI (SEQ ID NO: 1), hydrophobin (SEQ ID NO: 2), GAPDH-B (SEQ ID NO: 3), catalase B (SEQ ID NO: 5), and catalase A (SEQ ID NO: 6),
b. contacting said polypeptide with a putative binding partner, and
c. determining whether said putative binding partner is capable of binding to said polypeptide.

51. The method of claim 50, wherein the putative binding partner is a host-derived molecule.

52. The method of claim 50, wherein said method is repeated for a plurality of putative binding partners.

53. A method for identifying a compound with antifungal activity comprising the steps of:
a. providing a sensitized cell which has a reduced level of a polypeptide selected from the group consisting of: SEQ ID NOs: 1, 2, 3, 5, 6, and 36 and
b. determining the sensitivity of said cell to a putative antifungal compound, for instance by a growth assay.

54. A method for identifying an inhibitor of an extracellular Aspergillus polypeptide selected from the group consisting of: isopropylmalate dehydrogenase B (SEQ ID NO: 36), CssI (SEQ ID NO: 1), GAPDH (SEQ ID NO: 3), and catalase A (SEQ ID NO: 6), comprising the steps of:
a. providing two cells which differ in the level of a polypeptide selected from the group consisting of: isopropylmalate dehydrogenase B (SEQ ID NO: 36), CssI (SEQ ID NO: 1), GAPDH (SEQ ID NO: 3), and catalase A (SEQ ID NO: 6),
b. determining the sensitivity of said cells to a putative inhibitor, for instance by a growth assay, and
c. determining whether said two cells are differently affected by the presence of said putative inhibitor.

55. The method of claim 54, wherein the two cells differ in the copy number of said polypeptide.

56. The method of claim 54, wherein the two cells differ in the activity of said polypeptide.

57. A method of diagnosing fungal infection, preferably Aspergillus fumigatus, comprising the steps of:
a. providing a sample from an individual,
b. contacting said sample with an indicator moiety capable of specifically recognizing and binding a polypeptide selected from the group consisting of: isopropylmalate dehydrogenase B (SEQ ID NO:36), CssI (SEQ ID NO:1), hydrophobin (SEQ ID NO: 2), GAPDH-B (SEQ ID NO: 3), and catalase A (SEQ ID NO: 6), and
c. determining whether a signal has been generated by the indicator moiety.

58. The method of claim 57, wherein said indicator moiety comprises an antibody, such as an antibody as defined in claim 1.

59. A kit for the detection of fungal material, preferably intact fungal cells, most preferably intact Aspergillus fumigatus cells, in a biological sample comprising:
a. an indicator moiety capable of specifically recognizing and binding a polypeptide selected from the group consisting of: isopropylmalate dehydrogenase B (SEQ ID NO: 36), CssI (SEQ ID NO: 1), hydrophobin (SEQ ID NO: 2), GAPDH-B (SEQ ID NO: 3), and catalase A (SEQ ID NO: 6);
b. at least one buffer for promoting binding of the indicator moiety to the fungal material;
c. at least one reagent for generating a detectable signal; and
d. at least one written user instructions.

60. The kit of claim 59, wherein said indicator comprises an antibody, such as an antibody as defined in claim 1.

61. The antibody of claim 1, wherein the antibody is conjugated to a therapeutic moiety, such as a toxin or a fungicidal agent, or coupled to a detectable substance, such as a radioactive material.

62. The method of claim 27, wherein the method is combined with other antifungal therapy.

63. A host cell transformed or transfected with an expression vector as defined in claim 37.

64. A pharmaceutical composition comprising a polynucleotide as defined in claim 36 and a pharmaceutically-acceptable carrier.

65. A method for the immunization of a mammal against fungal infections, comprising the administration of a polynucleotide as defined in claim 31.


SEQUENCE LISTING (49 sequences)

SEQ ID NO: 1 - protein, 260 aa, Aspergillus Fumigatus
MLASFQFCILPRTYRTLLCSAGAGPLLIIQFVTVASALALAPTAVVARQGAAAFVTVNSIDVCPKKVAQEIINPGPKVVTTPYTCDQVKLGHGLDVSYYNFDIEPLTKDTFPYCKALKVFDNEGCLGFPTLWIPLESPLEDKCIPEHYFSDEVKSISFQLDCREDAPVKKEPYGPKEGAEQSAPQAEHSTKQDAQQGSHQGQEVQNSPKQEARQGSRPAEAAPKQEQEAEQASEAAPEKKASNPADSLGLGELTKVLGFR

SEQ ID NO: 2 - protein, 107 aa, Aspergillus Fumigatus
VRFPVPDDITVKQATEKCGDQAQLSCCNKATYAGDVTDIDEGILAGTLKNLIGGGSGTEGLGLFNQCSKLDLQSPIIGIPIQDLVNQKCKQNIACCQNSPSDAVRFP

SEQ ID NO: 3 - protein, 318 aa, Aspergillus Fumigatus
MATPKVGINGFGRIGRIVGLNSLSHGVDVVAVNDPFIEVHYAAYMLKYDTTHGQFKGTIETYDQGLIVNGKKIRFYAEKDPSQIPWSETGAAYIVESTGVFTTKEKASAHLKGGAKKVIISAPSADAPMFVMGVNNTTYTSDIQVLSNASCTTNCLAPLAKVINDKFGIVEGLMTTVHSYTATQKVVDAPSNKDWRGGRTAAQNIIPSSTGAAKAVGKVIPSLNGKLTGMAMRVPTSNVSVVDLTCRLEKGASYDEIKQAIKAASEEGELKNILGYTEDDVVSSDLNGDERSSIFDAKAGISLNPNFVKLVAWYDNEW

SEQ ID NO: 4 - protein, 438 aa, Aspergillus Fumigatus
MPISKIHARSVYDSRGNPTVEVDVATETGLHRAIVPSGASTGQHEAHELRDGDKTQWGGKGVLKAVKNVNETIGPALIKENIDVKDQSKVDEFLNKLDGTANKSNLGANAILGVSLAVAKAGAAEKGVPLYAHISDLAGTKKPYVLPVPFQNVLNGGSHAGGRLAFQEFMIVPDSAPSFSEALRQGAEVYQKLKALAKKKYGQSAGNVGDEGGVAPDIQTAEEALDLITEAIEQAGYTGKIKIAMDVASSEFYKADVKKYDLDFKNPESDPSKWLTYEQLADLYKSLAAKYPIVSIEDPFAEDDWEAWSYFYKTSDFQIVGDDLTVTNPGRIKKAIELKSCNALLLKVNQIGTLTESIQAAKDSYADNWGVMVSHRSGETEDVTIADIAVGLRSGQIKTGAPCRSERLAKLNQILRIEEELGENTVYAGSKFRTAVNL

SEQ ID NO: 5 - protein, 728 aa, Aspergillus Fumigatus
MRLTFIPSLIGVANAVCPYMTGELNRRDEISDGDAAAATEEFLSQYYLNDNDAFMTSDVGGPIEDQNSLSAGERGPTLLEDFIFRQKIQRFDHERVPERAVHARGAGAHGVFTSYGDFSNITAASFLAKEGKQTPVFVRFSTVAGSRGSSDLARDVHGFATRFYTDEGNFDIVGNNIPVFFIQDAILFPDLIHAVKPRGDNEIPQAATAHDSAWDFFSQQPSTMHTLLWAMSGHGIPRSFRHVDGFGVHTFRFVTDDGASKLVKFHWKSLQGKASMVWEEAQQTSGKNPDFMRQDLHDAIEAGRYPEWELGVQIMDEEDQLRFGFDLLDPTKIVPEEFVPITKLGKMQLNRNPRNYFAETEQVMFQPGHIVRGVDFTEDPLLQGRLFSYLDTQLNRHGGPNFEQLPINQPRVPVHNNNRDGAGQMFIPLNPHAYSPKTSVNGSPKQANQTVGDGFFTAPGRTTSGKLVRAVSSSFEDVWSQPRLFYNSLVPAEKQFVIDAIRFENANVKSPVVKNNVIIQLNRIDNDLARRVARAIGVAEPEPDPTFYHNNKTADVGTFGTKLKKLDGLKVGVLGSVQHPGSVEGASTLRDRLKDDGVDVVLVAERLADGVDQTYSTSDAIQFDAVVVAAGAESLFAASSFTGGSANSASGASSLYPTGRPLQILIDGFRFGKTVGALGSGTAALRNAGIATSRDGVYVAQSVTDDFANDLKEGLRTFKFLDRFPVDH

SEQ ID NO: 6 - protein, 749 aa, Aspergillus Fumigatus
MATKIAGGLHRAQEVLQNTSSKSKKLVDLERDTADAHTQQPLTTDHGVRVSNTDQWLRVTNDRRTGPSLLEDQIAREKIHRFDHERIPERVVHARGTGAFGNFKLKESIEDLTYAGVLTDTSRNTPVFVRFSTVQGSRGSADTVRDVRGFAVKFYTDEGNWDIVGNNIPVFFIQDAVKFPDFVHAVKPEPHNEVPQAQTAHNNFWDFVYLHPEATHMFMWAMSDRAIPRSYRMMQGFGVNTFALVNKEGKRHFVKFHWIPHLGVHSLVWDEALKLGGQDPDFHRKDLMEAIDNKAYPKWDFAIQVIPEEKQDDFEFDILDATKIWPENLVPLRVIGELELNRNVDEFFPQTEQVAFCTSHIVPGIDFTDDPLLQGRNFSYFDTQISRLGINWEELPINRPVCPVLNHNRDGQMRHRITQGTVNYWPNRFEAVPPTGTKGSGVGGGFTTYPQRVEGIKNRALNDKFREHHNQAQLFYNSMSEHEKLHMKKAFSFELDHCDDPTVYERLAGHRLAEIDLELAQKVAEMVGAPIPAKALKQNHGRRAPHLSQTEFIPKNPTIASRRIAIIIGDGYDPVASTGLKTAIKAASALPFIIGTKRSAIYATEDKTSSKGIIPDHHYDGQRSTMFDATFIPGGPHVATLRQNGQIKYWISETFGHLKALGATGEAVDLVKETLSGTLHVQVASSQSPEPVEWYGVVTAGGKQKPESFKESVQILKGATDFVGKFFYQISQHRNYQRELDGLASTIAF

SEQ ID NO: 7 - protein, 16 aa, Aspergillus Fumigatus
KVAQEIINPGPKVVTT

SEQ ID NO: 8 - protein, 16 aa, Aspergillus Fumigatus
KEGAEQSAPQAEHSTK

SEQ ID NO: 9 - protein, 17 aa, Aspergillus Fumigatus
PVPDDITVKQATEKCGD

SEQ ID NO: 10 - protein, 15 aa, Aspergillus Fumigatus
ATYAGDVTDIDEGIL

SEQ ID NO: 11 - protein, 16 aa, Aspergillus Fumigatus
TEDDVVSSDLNGDERS

SEQ ID NO: 12 - protein, 18 aa, Aspergillus Fumigatus
FKGTIETYDQGLIVNGKK

SEQ ID NO: 13 - protein, 17 aa, Aspergillus Fumigatus
KNVNETIGPALIKENID

SEQ ID NO: 14 - protein, 18 aa, Aspergillus Fumigatus
TSDFQIVGDDLTVTNPGR

SEQ ID NO: 15 - protein, 20 aa, Aspergillus Fumigatus
DEEDQLRFGFDLLDPTKIVP

SEQ ID NO: 16 - protein, 16 aa, Aspergillus Fumigatus
RIDNDLARRVARAIGV

SEQ ID NO: 17 - protein, 12 aa, Aspergillus Fumigatus
KVAQEIINPGPK

SEQ ID NO: 18 - protein, 10 aa, Aspergillus Fumigatus
FPVPDDITVK

SEQ ID NO: 19 - protein, 20 aa, Aspergillus Fumigatus
ATYAGDVTDIDEGILAGTLK

SEQ ID NO: 20 - protein, 11 aa, Aspergillus Fumigatus
AGISLNPNFVK

SEQ ID NO: 21 - protein, 15 aa, Aspergillus Fumigatus
TAAQNIIPSSTGAAK

SEQ ID NO: 22 - protein, 20 aa, Aspergillus Fumigatus
NILGYTEDDVVSSDLNGDER

SEQ ID NO: 23 - protein, 12 aa, Aspergillus Fumigatus
NVNETIGPALIK

SEQ ID NO: 24 - protein, 15 aa, Aspergillus Fumigatus
VNQIGTLTESIQAAK

SEQ ID NO: 25 - protein, 12 aa, Aspergillus Fumigatus
WLTYEQLADLYK

SEQ ID NO: 26 - protein, 11 aa, Aspergillus Fumigatus
VAQEIINPGPK

SEQ ID NO: 27 - protein, 10 aa, Aspergillus Fumigatus
FGFDLLDPTK

SEQ ID NO: 28 - protein, 9 aa, Aspergillus Fumigatus
SISFQLDCR

SEQ ID NO: 29 - protein, 15 aa, Aspergillus Fumigatus
EGAEQSAPQAEHSTK

SEQ ID NO: 30 - protein, 12 aa, Aspergillus Fumigatus
VVTTPYTCDQVK

SEQ ID NO: 31 - protein, 14 aa, Aspergillus Fumigatus
VPTSNVSVVDLTCR

SEQ ID NO: 32 - protein, 9 aa, Aspergillus Fumigatus
YDTTHGQFK

SEQ ID NO: 33 - protein, 15 aa, Aspergillus Fumigatus
GTIETYDQGLIVNGK

SEQ ID NO: 34 - protein, 12 aa, Aspergillus Fumigatus
TGPSLLEDQIAR

SEQ ID NO: 35 - protein, 172 aa, Aspergillus Fumigatus
SNASCTTNCLAPLAKVINDKFGIVEGLMTTVHSYTATQKVVDAPSNKDWRGGRTAAQNIIPSSTGAAKAVGKVIPSLNGKLTGMAMRVPTSNVSVVDLTCRLEKGASYDEIKQAIKAASEEGELKNILGYTEDDVVSSDLNGDERSSIFDAKAGISLNPNFVKLVAWYDNEW

SEQ ID NO: 36 - protein, 368 aa, Aspergillus Fumigatus
  VARIANT (1)...(368): Xaa = Any Amino Acid
MVTTYNILVLPGDGIGPEVMTEAVKVLKVFENEHRKFNLRQELIGGCSIDAHGKSVTEEVKKAALESDAVLFAAVGGPKWDHIRRGLDGPEGGLLQLRKAMDIYANLRPCSASSPSASIAKEFSPFRQEVIEGVDFVVVRENCGGAYFGKKIEEEDYAMDEWGYSEREIQRITRLXAEXALRHNPPWPVISLDKANVLASSRLWRRVVEKTMTTEYPQVKLVHQLADSASLILATNPRALNGVILADNTFGDMISDQAGSIVGTLGVLPSASLDGLPSETRKRTNGLYEPTHGSAPTIAGQNIANPVAMILCVALMFRYSLDMETEAQRIEKAVQGVLDAGIRTPDLGGKSGTNEVGDAIVAALQGSS

SEQ ID NO: 37 - protein, 8 aa, Aspergillus Fumigatus
  VARIANT (1)...(8): Xaa = Any Amino Acid
LXAEXALR

SEQ ID NO: 38 - DNA, 1226 bp, Aspergillus Fumigatus
  misc_feature (1)...(1226): n = A,T,C or G
atggtaactacttacaacatcctcgtcctccccggcgatgggatcggtcccgaggtcatgaccgaagcggtcaaggtgctaaaggtctttgagaacgagcaccgaaagttcaacctccggcaagagctcatcggcggttgcagcatcgatgcgcacggaaaatccgtcacagaagaagtgaaaaaggccgctctggaatccgacgccgtgctcttcgcagcagtcggaggtcccaaatgggaccatatccgtcgtggtcttgacgggccggagggaggcctgctgcagctccgcaaggcgatggacatctacgcgaatctcaggccgtgctcggccagttcgccgagtgcgtcgatcgcgaaggagtttagcccattccgccaggaagtgatcgagggcgtagatttcgtcgtggtgagggagaactgcgggggagcgtatttcgggaagaagatcgaagaagaagattatggtacgtcgtttttaacaagcagtatgctttcgagactgactgtgttatttcagcgatggacgaatggggctatagcgagcgcgagatccagcgcatcacccgcctcnnngcggaannngccctccgtcacaaccccccctggcccgtcatctccctggacaaagccaatgtgctcgcctcgtcgcggctctggcggcgcgtcgttgaaaagaccatgaccactgagtatccccaggtgaagctcgtgcaccagctggcagactcagcatcgctgattctagcgaccaacccgcgggcattgaacggtgtcatcttggctgacaacacattcggcgacatgatttctgaccaggccggttccatcgtcgggacattgggcgtgcttcccagtgccagtctcgatggactacccagtgaaacaagaaagcggacaaatggtctgtacgagccgacccatggatctgcaccgacgtacgtttcttcctttgttacccgaattatcatgtttcactgaagcaagctgacaatcatctgcagaattgcgggccagaacatcgccaaccccgttgccatgatcctctgtgtggctctcatgttccgctattcgctagacatggagaccgaggcgcaacggatcgaaaaagcagtgcagggtgttcttgatgccgggatccgcacccctgatctgggtgggaaatcggggacgaatgaagttggggatgcaattgttgctgcgttgcagggtagttcataa

SEQ ID NO: 39 - DNA, 1107 bp, Aspergillus Fumigatus
  misc_feature (1)...(1107): n = A,T,C or G
atggtaactacttacaacatcctcgtcctccccggcgatgggatcggtcccgaggtcatgaccgaagcggtcaaggtgctaaaggtctttgagaacgagcaccgaaagttcaacctccggcaagagctcatcggcggttgcagcatcgatgcgcacggaaaatccgtcacagaagaagtgaaaaaggccgctctggaatccgacgccgtgctcttcgcagcagtcggaggtcccaaatgggaccatatccgtcgtggtcttgacgggccggagggaggcctgctgcagctccgcaaggcgatggacatctacgcgaatctcaggccgtgctcggccagttcgccgagtgcgtcgatcgcgaaggagtttagcccattccgccaggaagtgatcgagggcgtagatttcgtcgtggtgagggagaactgcgggggagcgtatttcgggaagaagatcgaagaagaagattatgcgatggacgaatggggctatagcgagcgcgagatccagcgcatcacccgcctcnnngcggaannngccctccgtcacaaccccccctggcccgtcatctccctggacaaagccaatgtgctcgcctcgtcgcggctctggcggcgcgtcgttgaaaagaccatgaccactgagtatccccaggtgaagctcgtgcaccagctggcagactcagcatcgctgattctagcgaccaacccgcgggcattgaacggtgtcatcttggctgacaacacattcggcgacatgatttctgaccaggccggttccatcgtcgggacattgggcgtgcttcccagtgccagtctcgatggactacccagtgaaacaagaaagcggacaaatggtctgtacgagccgacccatggatctgcaccgacgattgcgggccagaacatcgccaaccccgttgccatgatcctctgtgtggctctcatgttccgctattcgctagacatggagaccgaggcgcaacggatcgaaaaagcagtgcagggtgttcttgatgccgggatccgcacccctgatctgggtgggaaatcggggacgaatgaagttggggatgcaattgttgctgcgttgcagggtagttcataa

SEQ ID NO: 40 - DNA, 1093 bp, Aspergillus Fumigatus
atgccgtcatataacattgtcgttttcgctggggaccactgtggtccggaggtaagttcggtcctgcgcgtcatcgagaagtgccgtgacgatgctaccttcaacctccaggatcaattgctcggtggtgtaagttcgatcgatgctaccggatctccccttaccgacgaagctcttaacgccgcaaagaacgccgatgccgttctcctcggtgccattggcggtcccaaatggggcactggcgccgtccgccccgaacagggcctcctccgtctgcgcaaggagatgggcacattcggtaacctccgcccctgcaacttcgccgccccgtcgctggtcgacggctcccctctccgccccgaagtctgccgcggcgtcgacttcaacattatccgcgaactgaccggtggcatctacttcggcgaccgcaaggaggacgacggcagcggcttcgccatggacacggagccgtactcccgcgcggagatcgagcgcatcacccgccttgcggcccacctcgctctgcagcacaacccccctcttcccgtgtggagcttggacaaggccaacgtcctcgcgacgagccggctgtggcggaagaccgtgacggaggtcatggccaaggagttcccccagctcaaggtggagcaccagctcattgactccgcggccatgatcatggtcaaggagcctagaaagcttaacggtattgttgtcactagcaacctgttcggtgacatcatcagtgatgaagccagcgttatccctggttctctgggactcttgcccagcgcaagcttgagcggcattcctgacggaaagaccaaggtcaatggtatctatgagcctattcacggttctgcccctgacattgccggcaagggcatcgttaaccccgtcgccgccattctctctgtcgccatgatgatgcagtactccctgaaccgtatggatgacgccagggccatcgagacggccgtccgcaatgtgatcgaggccggtatccgcactgccgatattggcggcaagtcgacaactagcgaggtcggtgacgctgttgctgccgagctggagaagctgttgaagcaatagt

SEQ ID NO: 41 - protein, 363 aa, Aspergillus Fumigatus
MPSYNIVVFAGDHCGPEVSSVLRVIEKCRDDATFNLQDQLLGGVSSIDATGSPLTDEALNAAKNADAVLLGAIGGPKWGTGAVRPEQGLLRLRKEMGTFGNLRPCNFAAPSLVDGSPLRPEVCRGVDFNIIRELTGGIYFGDRKEDDGSGFAMDTEPYSRAEIERITRLAAHLALQHNPPLPVWSLDKANVLATSRLWRKTVTEVMAKEFPQLKVEHQLIDSAAMIMVKEPRKLNGIVVTSNLFGDIISDEASVIPGSLGLLPSASLSGIPDGKTKVNGIYEPIHGSAPDIAGKGIVNPVAAILSVAMMMQYSLNRMDDARAIETAVRNVIEAGIRTADIGGKSTTSEVGDAVAAELEKLLKQ

SEQ ID NO: 42 - DNA, 18 bp, Aspergillus Fumigatus
atgcctatctccaagatc

SEQ ID NO: 43 - DNA, 15 bp, Aspergillus Fumigatus
caggttgacggcagt

SEQ ID NO: 44 - DNA, 18 bp, Aspergillus Fumigatus
atggtaactacttacaac

SEQ ID NO: 45 - DNA, 18 bp, Aspergillus Fumigatus
tgaactaccctgcaacgc

SEQ ID NO: 46 - DNA, 1233 bp, Aspergillus Fumigatus
atgggttctggatccggtgatgacgatgacaagctcgcccttatggtaactacttacaacatcctcgtcctccccggcgatgggatcggtcccgaggtcatgaccgaagcggtcaaggtgctaaaggtctttgagaacgagcaccgaaagttcaacctccggcaagagctcatcggcggttgcagcatcgatgcgcacggaaaatccgtcacagaagaagtgaaaaaggccgctctggaatccgacgccgtgctcttcgcagcagtcggaggtcccaaatgggaccatatccgtcgtggtcttgacgggccggagggaggcctgctgcagctccgcaaggcgatggacatctacgcgaatctcaggccgtgctcggccagttcgccgagtgcgtcgatcgcgaaggagtttagcccattccgccaggaagtgatcgagggcgtagatttcgtcgtggtgagggagaactgcgggggagcgtatttcgggaagaagatcgaagaagaagattatgcgatggacgaatggggctatagcgagcgcgagatccagcgcatcacccgcctctcggcggaaattgccctccgtcacaaccccccctggcccgtcatctccctggacaaagccaatgtgctcgcctcgtcgcggctctggcggcgcgtcgttgaaaagaccatgaccactgagtatccccaggtgaagctcgtgcaccagctggcagactcagcatcgctgattctagcgaccaacccgcgggcattgaacggtgtcatcttggctgacaacacattcggcgacatgatttctgaccaggccggttccatcgtcgggacattgggcgtgcttcccagtgccagtctcgatggactacccagtgaaacaagaaagcggacaaatggtctgtacgagccgacccatggatctgcaccgacaattgcgggccagaacatcgccaaccccgttgccatgatcctctgtgtggctctcatgttccgctattcgctagacatggagaccgaggcgcaacggatcgaaaaagcagtgcagggtgttcttgatgccgggatccgcacccctgatctgggtgggaaatcggggacgaatgaagttggggatgcaattgttgctgcgttgcagggtagttcaaagggcgagcttgaaggtaagcctatccctaaccctctcctcggtctcgattctacgcgtaccggtcatcatcaccatcaccattga

SEQ ID NO: 47 - protein, 410 aa, Aspergillus Fumigatus
MGSGSGDDDDKLALMVTTYNILVLPGDGIGPEVMTEAVKVLKVFENEHRKFNLRQELIGGCSIDAHGKSVTEEVKKAALESDAVLFAAVGGPKWDHIRRGLDGPEGGLLQLRKAMDIYANLRPCSASSPSASIAKEFSPFRQEVIEGVDFVVVRENCGGAYFGKKIEEEDYAMDEWGYSEREIQRITRLSAEIALRHNPPWPVISLDKANVLASSRLWRRVVEKTMTTEYPQVKLVHQLADSASLILATNPRALNGVILADNTFGDMISDQAGSIVGTLGVLPSASLDGLPSETRKRTNGLYEPTHGSAPTIAGQNIANPVAMILCVALMFRYSLDMETEAQRIEKAVQGVLDAGIRTPDLGGKSGTNEVGDAIVAALQGSSKGELEGKPIPNPLLGLDSTRTGHHHHHH

SEQ ID NO: 48 - DNA, 1443 bp, Aspergillus Fumigatus
atgggctctggatccggtgatgacgatgacaagctcgcccttatgcctatctccaagatccacgctcgttccgtgtacgactctcgcggtaaccccaccgttgaggtggacgttgtcaccgagaccggtttgcaccgtgctattgttccttctggagcttccaccggccagcacgaggctcacgagctccgtgacggtgataagacccagtggggcggcaagggtgtcctcaaggctgtcaagaatgtcaacgagaccattggccctgctctcatcaaggagaacatcgatgtgaaggaccagtctaaggtcgacgagttccttaacaagcttgacgggactgccaacaagtccaacctcggtgctaatgccatcctcggtgtcagcttggctgttgccaaggctggtgctgctgagaagggtgtccctctctacgctcacatctccgaccttgccggtaccaagaagccctatgtccttcccgttcccttccagaacgtcctgaacggcggctctcacgccggtggtcgcctcgctttccaggagttcatgatcgtccctgactccgctccctctttctccgaggccctccgccagggtgctgaggtctaccagaagctcaaggctctggccaagaagaagtacggccagtccgctggcaacgttggtgacgagggtggtgttgctcccgatattcagaccgccgaggaggctctcgacctgatcaccgaggccatcgagcaggccggctacaccggcaagatcaagatcgctatggacgttgcctccagcgagttctacaaggccgacgtcaagaagtacgaccttgacttcaagaaccccgagagcgacccctccaagtggctcacctacgagcagcttgccgacctctacaagtcccttgctgccaagtaccccattgtcagcattgaggaccccttcgctgaggatgattgggaggcctggagctacttctacaagacctccgacttccagattgttggtgatgacctgactgttactaaccctgggcgtatcaagaaggccatcgagctcaagtcctgcaacgccctcctgctcaaggtcaaccagatcggtaccctcaccgagtccatccaggccgccaaggactcctacgccgacaactggggtgtcatggtctcccaccgctctggtgagactgaggacgtcaccattgccgacattgctgtcggtctgcgctctggccagatcaagaccggtgctccttgccgttccgagcgtctggctaagctgaaccagatcctccgtatcgaggaggagctcggcgagaatgccgtctacgctggttccaagttccgcactgccgtcaacctgaagggcgagcttgaaggtaagcctatccctaaccctctcctcggtctcgattctacgcgtaccggtcatcatcaccatcaccattga

SEQ ID NO: 49 - protein, 480 aa, Aspergillus Fumigatus
MGSGSGDDDDKLALMPISKIHARSVYDSRGNPTVEVDVVTETGLHRAIVPSGASTGQHEAHELRDGDKTQWGGKGVLKAVKNVNETIGPALIKENIDVKDQSKVDEFLNKLDGTANKSNLGANAILGVSLAVAKAGAAEKGVPLYAHISDLAGTKKPYVLPVPFQNVLNGGSHAGGRLAFQEFMIVPDSAPSFSEALRQGAEVYQKLKALAKKKYGQSAGNVGDEGGVAPDIQTAEEALDLITEAIEQAGYTGKIKIAMDVASSEFYKADVKKYDLDFKNPESDPSKWLTYEQLADLYKSLAAKYPIVSIEDPFAEDDWEAWSYFYKTSDFQIVGDDLTVTNPGRIKKAIELKSCNALLLKVNQIGTLTESIQAAKDSYADNWGVMVSHRSGETEDVTIADIAVGLRSGQIKTGAPCRSERLAKLNQILRIEEELGENAVYAGSKFRTAVNLKGELEGKPIPNPLLGLDSTRTGHHHHHH